(19) United States
(12) Patent Application Publication
(10) Pub. No.: US 2022/0348930 A1
(43) Pub. Date: Nov. 3, 2022

(54) CAR NKTs Expressing Artificial Micro RNA-Embedded shRNA for Downregulation of MHC Class I & II Expression

(71) Applicant: Baylor College of Medicine, Houston, TX (US)

(72) Inventors: Andras HECZEY, Houston, TX (US); Bin LIU, Richmond, TX (US); Leonid METELITSA, Sugar Land, TX (US)

(73) Assignee: Baylor College of Medicine, Houston, TX (US)

(21) Appl. No.: 17/727,528

(22) Filed: Apr. 22, 2022

Related U.S. Application Data
(60) Provisional application No. 63/179,104, filed on Apr. 23, 2021.

(51) Int. Cl. C12N 15/113; C07K 16/28; C07K 14/54; C07K 14/55; C12N 5/0783; A61P 35/00; A61K 35/17; A61P 37/06; C07K 16/30

(52) U.S. Cl. CPC C12N 15/1138; C07K 16/2803; C07K 14/5443; C07K 14/55; C12N 5/0646; A61P 35/00; A61K 35/17; A61P 37/06; C07K 16/3084; C07K 16/303; C12N 2310/122; C12N 2310/141; C07K 2319/33; C07K 2317/76; A61K 2039/505

(57) ABSTRACT

The present disclosure provides methods and compositions related to Natural Killer T cells that are engineered to knock down the expression of one or more endogenous major histocompatibility complex (MHC) gene. The present disclosure also provides engineered CAR NKT cells that resist rejection by allogeneic immune cells both in vitro and in vivo.

### STATEMENT REGARDING FEDERALLY SPONSORED RESEARCH OR DEVELOPMENT

This invention was made with government support under Grant No. 5 P50 CA126752 awarded by the National Institutes of Health. The government has certain rights in the invention.

### INCORPORATION OF SEQUENCE LISTING

The sequence listing that is contained in the file named “P35062US_ST25,” which is 7,266 bytes (measured in operating system MS-Windows), recorded on Apr. 22, 2022, is filed herewith and incorporated herein by reference.

### FIELD

The present disclosure relates to at least the fields of cell biology, molecular biology, immunology, and medicine.

### BACKGROUND

Type-I NKT cells (NKTs) are an evolutionary conserved subset of innate lymphocytes that express invariant TCRα-chain Vα24-Jα18 and react to self- or microbial-derived glycolipids presented by monomorphic HLA class-I like molecule CD1d (Gene ID 912) (Porcelli et al. Analysis of T cell antigen receptor (TCR) expression by human peripheral blood CD4-8-alpha/beta T cells demonstrates preferential use of several V beta genes and an invariant TCR alpha chain. J. Exp. Med. 1993; 178(1):1-16); Lantz and Bendelac, “An invariant T cell receptor alpha chain is used by a unique subset of major histocompatibility complex class I-specific CD4+ and CD4-8− T cells in mice and humans,” J. Exp. Med. 1994; 180(3): 1097-1106; Bendelac A, Lantz O, Quimby M E, Yewdell J W, Bennink J R, Brutkiewicz R R. CD1 recognition by mouse NK1+ T lymphocytes. Science 1995; 268(5212):863-865; Kim E Y, Lynch L, Brennan P J, Cohen N R, Brenner M B. The transcriptional programs of iNKT cells. Semin. Immunol. 2015; 27(1):26-32).

Global transcriptional profiling studies demonstrate that NKTs, though they share properties with T and NK cells, are a distinct population of lymphocytes (Cohen et al., 2013). Both in mice and humans, NKTs diverge from conventional T cells at the stage of CD4+CD8+ (double positive, DP) thymocytes (CD8, Gene ID 925). Unlike conventional T cells, which are positively selected by thymic epithelial cells, NKTs are selected by CD1d-expressing DP thymocytes (Gapin L, Matsuda J L, Surh C D, Kronenberg M. NKT cells derive from double-positive thymocytes that are positively selected by CD1d. Nat. Immunol. 2001; 2(10):971-978). The expression of promyelocytic leukemia zinc finger transcription factor (PLZF) immediately after positive selection enables intrathymic expansion and effector/memory-like differentiation of NKTs (Savage A K, et al. The transcription factor PLZF directs the effector program of the NKT cell lineage. Immunity. 2008; 29(3):391-403).

NKT cells have numerous anti-tumor properties and their numbers have been reported to correlate with good outcome in several types of cancer. Heczey A. et al. and Tian G. et al. demonstrated that NKT cells can be isolated from peripheral blood, transduced with a CAR and expanded to clinical scale for adoptive cell therapy applications. Several studies have shown that donor-derived NKTs do not mediate GvHD and even may suppress it. Therefore, allogeneic healthy donor-derived CAR-NKT cells could be used to treat cancer patients without a risk of GvHD that, in contrast to T cells, does not require additional genetic manipulation.

All normal nucleated cells however express HLA class I and therefore adoptively transferred therapeutic cells from HLA mismatched donors will be eliminated by the host immune system. T and NKT cells can also transiently express HLA class II when activated, and HLA class II mismatch triggers donor cell elimination by host CD4 T cells. A common approach to delay such rejection is to use of immunosuppressive host conditioning to allow a therapeutic window for effector cells to mediate anti-tumor activity before recovery of the host immune system. However, such approach is toxic to patients and may not allow complete tumor control due to insufficient persistence of the therapeutic effector cells.

There is therefore a need for off-the-shelf CAR-based cellular immunotherapies that can be rapidly expanded to clinical scale, do not induce graft-versus-host disease (GvHD), and are tolerated by patients. Due to restriction by monomorphic CD1d, NKT cells do not produce GvHD.

To limit rejection of CAR-NKT cells by the immune system of an allogeneic host, the instant disclosure provides constructs that incorporate shRNA sequences against β2-microglobulin (B2M) and the invariant chain (Ii) (a.k.a. CD74) or the class II transactivator (CIITA) to achieve knock-down of HLA class I and class II, respectively, in NKT cells. In particular, the instant disclosure provides constructs comprising embedded shRNA sequences within an artificial microRNA (amiR) scaffold integrated into the CAR construct.

Here it is shown that optimized CAR-amiR constructs mediate effective knockdown of HLA class I and II in transduced NKT cells. NKT cells expressing these constructs demonstrate potent in vivo anti-tumor activity in a lymphoma NSG mouse model and resist rejection by allogeneic immune cells both in vitro and in vivo.

### SUMMARY

The present disclosure provides for, and includes, a recombinant construct for suppressing the expression of an endogenous major histocompatibility complex (MHC) gene, comprising a DNA sequence encoding a chimeric antigen receptor (CAR) recognizing a tumor antigen and a DNA sequence encoding a small hairpin RNA (shRNA) sequence targeting an MHC class I or MHC class II gene, where the shRNA sequence is embedded in an artificial microRNA (amiR) scaffold.

In one aspect, the recombinant construct as disclosed herein further comprises a DNA sequence encoding a cytokine. In some aspects, the cytokine is interleukin-15 (IL-15), IL-7, IL-12, IL-18, IL-21, IL-27, IL-33, or a combination thereof. In one aspect, the cytokine is IL-15. In one aspect, the IL-15 is a human IL-15. In one aspect, the DNA sequence encoding an IL-15 is codon-optimized. In another aspect, the IL15 comprises an IL-2 signal peptide.

In some aspects, the amiR is amiR155. In other aspects, the amiR is amiR30.

In some aspects, the MHC class I gene encodes a β2-microglobulin (B2M).

In some aspects, the MHC class II gene encodes an invariant chain (Ii) or a class II transactivator (CIITA).

In some aspects, the recombinant constructs as disclosed herein comprise a first shRNA sequence embedded in a first amiR scaffold and a second shRNA sequence embedded in a second amiR scaffold. In some aspects, the first shRNA sequence targets a MHC class I gene and the second shRNA sequence targets a MHC class I gene. In one aspect, the first amiR scaffold and the second amiR scaffold are from the same amiR sequence. In other aspects, the first amiR scaffold and the second amiR scaffold are from different amiR sequences.

The present disclosure also provides for, and includes, a method for limiting rejection of an engineered natural killer T (NKT) cell by the immune system of an allogeneic host, comprising transducing an NKT cell with the recombinant constructs disclosed herein, where the expression of the endogenous MHC gene in the NKT cell is suppressed by the shRNA.

In some aspects, the expression level of the endogenous MHC gene is decreased by at least 10% 2 days post-transduction.

In some aspects, the expression level of the endogenous MHC gene is decreased by at least 10% 7 days post-transduction.

In some aspects, the expression level of the endogenous MHC gene is decreased by at least 10% 14 days post-transduction.

In some aspects, the NKT cell is a CD1d-restrictive NKT cell.

The present disclosure further provides for, and includes, an engineered NKT cell transduced with the recombinant constructs as disclosed herein, or produced by a method disclosed herein, where the expression of the endogenous MHC gene in the NKT cell is significantly suppressed compared with a control NKT cell not transduced with the recombinant construct.

In some aspects, the engineered NKT cell has improved resistance to rejection by allogeneic T cells or PBMCs.

In some aspects, the engineered NKT cell has improved resistance to destruction by allogeneic natural killer cells.

### BRIEF DESCRIPTION OF THE DRAWINGS

The present disclosure is disclosed with reference to the accompanying drawings, wherein:

FIG. 1 presents a diagram of CAR19 expression constructs with artificial microRNA (amiR) (CAR19-amiR) or pol III promoter-based expression of short hairpin RNA or small hairpin RNA (shRNA) (CAR19-shRNA) sequences against β2-microglobulin (B2M) and the invariant chain (Ii) (a.k.a. CD74) or the class II transactivator (CIITA). LTR=long terminal repeat, scFv=single chain variable fragment, H=hinge, TM=transmembrane. In some embodiments, the U6 promoter is replaced with an H1 or 7SK promoter.

FIG. 2 presents representative results of CAR19 expression in NKTs are transduced with CAR19 constructs containing scrambled (scr.) or B2M-specific shRNA driven by the U6, H1, or 7SK promoter or embedded in the miR155 scaffold. CAR expression is evaluated 2 days post-transduction.

FIG. 3 presents a representative dot plot of intracellular flow cytometry of a donor gating the cells into CAR19 and HLA-A,B,C of NKTs transduced with CAR19 constructs containing scrambled (scr.) or B2M-specific shRNA driven by the H1, 7SK, or U6 promoters or embedded in amiR155 as indicated. Representative histograms of HLA-A,B,C expression for transduced and non-transduced samples is shown for each. B2M shRNA expression supported by amiR155 from within CAR19 is shown to result in the greatest level of knockdown of HLA-A,B,C (bottom right). CAR and HLA-A,B,C expression is evaluated 2 days post-transduction.

FIG. 4 presents another representative dot plot of intracellular flow cytometry of a donor gating the cells into CAR19 and HLA-A,B,C of NKTs transduced with CAR19 constructs containing scrambled (scr.) or B2M-specific shRNA driven by the U6 promoter or embedded in amiR155 as indicated. CAR and HLA-A,B,C expression is evaluated 14 days post-transduction.

FIG. 5 presents a representative dot plot of intracellular flow cytometry of a donor gating the cells into CAR19 and HLA-A,B,C of NKTs transduced with CAR19 constructs containing scrambled (scr.) or B2M-specific shRNA embedded in amiR30 as indicated. CAR and HLA-A,B,C expression is evaluated 7 days post-transduction.

FIG. 6 presents representative dot plots of intracellular flow cytometry of a donor gating the cells into CAR19 and HLA-A,B,C of NKTs transduced with CAR19 constructs containing 5 different B2M-specific shRNA sequences (SEQ ID NOs:1 to 5) embedded in amiR155 and previously evaluated shRNA sequence (SEQ ID NO:6) used in ANCHOR product. CAR and HLA-A,B,C expression is evaluated 12 days post-transduction. The results are quantified and presented in Table 4.

FIGS. 7A to 7C present representative dot plots of intracellular flow cytometry of a donor gating the cells into CAR19 and HLA-DR,DP,DQ of NKTs transduced with ten CAR19 constructs containing CIITA-specific shRNA (SEQ ID NOs:7 to 16 corresponding to graphs 1 to 10 respectively) embedded in amiR155. CAR and HLA-DR,DP,DQ expression is evaluated 12 days post-transduction. The results are quantified and presented in Table 4.

FIGS. 8A to 8C present representative dot plots of intracellular flow cytometry of a donor gating the cells into CAR19 and HLA-DR,DP,DQ of NKTs transduced with ten CAR19 constructs containing CD74-specific shRNA (SEQ ID NOs:17 to 26 corresponding to graphs 1 to 10 respectively) embedded in amiR155. CAR and HLA-DR,DP,DQ expression is evaluated 12 days post-transduction. The results are quantified and presented in Table 4.

FIG. 9 presents a representative plot of the percent knockdown of NKTs transduced with CAR19.15 constructs containing single amiR-embedded shRNA targeting B2M (SEQ ID NO:X or CIITA (SEQ ID NO:12) as indicated. Knockdown efficiency was evaluated four days post-transduction. N=4 donors.

FIG. 10 presents a graph of IL-15 secretion from representative donor NKT cells transduced with the indicated constructs using the BioLegend ELISA MAX™ Deluxe Set Human IL-15 kit (BioLegend #435104) and expression of CAR19. FIG. 10 panel A presents NKT cells transduced with CAR19.15, CAR19.15.u6-b2m, car19.15.miR155-b2m, or non-transduced (NT) and either cultured alone or co-cultured with CD19-positive Raji lymphoma cells for 48 hours. FIG. 10 panel B presents NKT cells transduced with CAR19 constructs containing B2M-specific shRNA driven by the U6 promoter or embedded in the miR155 scaffold. CAR expression is evaluated two days post-transduction. N=1 donor, three technical repeats.

FIG. 11 presents diagrams of constructs designed to boost IL15 expression from knockdown constructs by incorporation of codon-optimized IL15 sequence, IL15 receptor alpha (IL15Ra), and IL15Ra Sushi domain (extracellular N terminal portion of IL15Ra, essential for binding IL15).

FIG. 12 panel A and panel B present graphs of IL-15 expression of NKTs transduced with the indicated constructs or non-transduced and either cultured alone or co-cultured with CD19-positive Raji lymphoma cells for 72 hours. Culture supernatant are processed using the BioLegend ELISA MAX™ Deluxe Set Human IL-15 kit (BioLegend #435104) to detect IL15 secretion. A) N=1 donor, three technical repeats. B) N=3 donors.

FIG. 13 presents representative dot plots of intracellular flow cytometry of a donor gating the cells into CAR19 and IL-15 of NKTs transduced with CAR19.15-15Ra-amiR-B2M construct (FIG. 11) and IL15 expression is evaluated four days later. Data shown from three donors.

FIG. 14 presents a diagram of a double knockdown construct of a CAR19 and codon-optimized IL15 expression paired amiR30-B2M shRNA and amiR155-CIITA shRNA to mediate HLA class I and II knockdown, respectively.

FIGS. 15A and 15B present representative dot plots (A) of intracellular flow cytometry of a donor gating the cells into CAR19 and HLA-A,B,C or HLA-DR,DP,DQ of NKTs transduced with the CAR19 construct shown in FIG. 14 and a graph of knockdown percentage (B) for three donors (BL #81, 82, 83).

FIGS. 16A and 16B present representative dot plots from four donors of intracellular flow cytometry of a donor gating the cells into CAR19 and HLA-A,B,C or HLA-DR,DP,DQ of NKTs transduced with the CAR19 construct shown in FIG. 14. CAR, HLA-A,B,C, and HLA-DR,DP,DQ expression are evaluated at day 19 of expansion. Labels indicate MFI for each population and knock-down percentage between cell populations connected by arrows.

FIG. 17 presents a representative graph of IL15 secretion in NKT cells transduced with the indicated constructs or non-transduced and either cultured alone or co-cultured with CD19-positive Raji lymphoma cells for 48 hours. The culture supernatant is processed using the BioLegend ELISA MAX™ Deluxe Set Human IL-15 kit (BioLegend #435104) to detect IL15 secretion. N=3 donors (BL #81, 82, 83).

FIG. 18 presents a representative graph of in vitro cytotoxicity against CD19-positive target cells compared with CAR19 and CAR19.IL15 NKT cells. NKT cells are transduced with indicated constructs and co-cultured for six hours with CD19-positive Raji lymphoma cells engineered to express high levels of firefly luciferase at specified effector-to-target ratios. Luciferin was added at the conclusion of the assay for detection of bioluminescence.

FIGS. 19A and 19B present results of NKT cells transduced with CAR19.opti-IL15 double knockdown constructs to control CD19-positive tumors in vivo and promote survival of NSG mice comparably to CAR19.15 NKT cells. FIG. 19A presents imaging of NSG mice injected intravenously with 2×10⁵ firefly luciferase-positive Daudi lymphoma cells on day 0 followed by intravenous injection of 5×10⁶ NKT cells transduced with indicated constructs or no construct (non-transduced, NT) on day 3. Just prior to imaging, each mouse receives 100 luciferin at 30 mg/mL via intraperitoneal injection and are imaged under a bioluminescent channel. (Bioluminescent counts scale 600-30,000) FIG. 19B presents a Kaplan Meier survival curve for the mice shown in FIG. 19A.

FIG. 20 presents a diagram of a double knockdown construct of a CAR19 and codon-optimized IL15 containing a fused IL2 signal peptide (IL2SP) to boost IL15 secretion. SD/SA=splice donor/splice acceptor

FIG. 21 presents a representative graph of IL15 secretion by NKT cells expressing the double knockdown construct of FIG. 20. NKT cells are transduced with the indicated constructs or non-transduced and either cultured alone or co-cultured with CD19-positive Raji lymphoma cells for 48 hours. The culture supernatant is processed using the BioLegend ELISA MAX™ Deluxe Set Human IL-15 kit (BioLegend #435104) to detect IL15 secretion.

FIG. 22 presents results of NKT cells transduced with the IL2SP-opti IL15 CAR19 construct with double amiR knockdown of FIG. 20 to control CD19-positive tumors in vivo and promote survival of NSG mice comparably to CAR19.15 NKT cells. NSG mice are injected intravenously with 2×10⁵ firefly luciferase-positive Daudi lymphoma cells on day 0 followed by intravenous injection of 1×10⁶ or 5×10⁶ NKTs transduced with indicated constructs or no construct (non-transduced, NT) on day 4. Just prior to imaging, each mouse receives 100 μL luciferin at 30 mg/mL via intraperitoneal injection and are imaged under a bioluminescent channel. Bioluminescent counts scale 600-30,000.

FIGS. 23A and 23B present results of tumor progression in NSG mice treated with CAR NKT cells expressing double knockdown construct and a Kaplan Meier survival curve respectively. NSG mice are injected intravenously with 2×10⁵ firefly luciferase-positive Daudi lymphoma cells on day 0 followed by intravenous injection of 5×10⁶ NKTs transduced with indicated constructs or no construct (non-transduced, NT) on day 3. Just prior to imaging, each mouse receives 100 μL luciferin at 30 mg/mL via intraperitoneal injection and are imaged under a bioluminescent channel. Bioluminescent counts scale 2000-30,000. B) Kaplan Meier survival curve for mice shown in A). Tumor progression is delayed and survival is unchanged.

FIG. 24 presents a representative graph of NKT cells expressing 1) CAR19.15 containing two scrambled shRNA sequences in place of B2M and CIITA (CAR19.IL2SP-opti15.amiR-SCR-amiR-SCR, scramble), 2) CAR19.15 with amiR-embedded B2M and CIITA shRNA sequences (CAR19.IL2SP-opti15.amiR-B2M-amiR-CIITA, knockdown), and the B2M/CIITA double knockdown construct (knockout) evaluated by flow cytometry daily for CAR and HLA expression, gated on HLA I-cells. Recipient NK cells (HLA-A2+) are isolated using the NK cell isolation kit (Miltenyi Biotech) and co-cultured with donor NKT cells (HLA-A2−) at a 1:1 ratio for three days. NKT cells expressing the B2M/CIITA double knockdown construct persist in the presence of allogeneic NK cells while double knock-out leaves NKT cells vulnerable to NK cell killing.

FIG. 25 presents representative graphs of flow cytometry of NKT cells transduced with scrambled, knockdown, and knockout constructs of FIG. 24 every 2 to 3 days. Pan T cells are isolated from recipient PBMCs using the naive pan T cell isolation kit, human (Miltenyi Biotech. Recipient T cells (HLA-A2+) are co-cultured with donor NKT cells (HLA-A2−) at a 2:1 (T:NKT) ratio for seven days. NKT cells expressing the B2M/CIITA double knockdown construct resist rejection by allogeneic T cells compared to NKT cells carrying scrambled shRNA control construct.

FIG. 26 presents representative graphs of flow cytometry results of transduced NKT cells evaluated every 2 to 3 days in co-culture with allogenic PBMCs. Recipient PBMCs (HLA-A2+) are co-cultured with donor NKT cells (HLA-A2−) at a 10:1 (PBMC:NKT) ratio for seven days. NKT cells are transduced with 1) CAR19.15 with scrambled shRNA control, or 2) CAR19.15 with double knockdown.

FIGS. 27A and 27B present representative graphs of flow cytometry of NKT cells transduced with 1) CAR19.IL2SP-opti15 with scrambled shRNA sequences in place of B2M control (Scr), 2) CAR19.IL2SP-opti15 with double knockdown (KD), 3) CAR19.IL2SP-opti15 with double knockout (KO) and co-cultured with recipient NK cells (HLA-A2+) isolated using the NK cell isolation kit (Miltenyi Biotech) at a 2:1 (NK:NKT) ratio for two days. Panel A of FIG. 27A presents representative flow plots showing total frequency of donor NKT cells on day 0 and day 2 of co-culture (top, FIG. 27B). Panel B of FIG. 27A presents absolute cell counts of donor NKT cells and Panel C of FIG. 27B present recipient NK cells on day 0 and day 2 of co-culture. All data denote mean±s.d., three unique donor-recipient pairs are used. P values are determined using two-way ANOVA with Sidak's correction for multiple comparisons and nonsignificant (P>0.05) values are not shown. P values are determined using the two-tailed, paired Student's t-test.

FIGS. 28A and 28B present representative graphs of flow cytometry of transduced NKT cells and absolute cell counts of donor NKT cells and recipient T cells in another aspect. Pan T cells are isolated from recipient PBMCs (HLA-A2+) using the naive pan T cell isolation kit, human (Miltenyi Biotech). Purified T cells are then stimulated with OKT3/αCD28 for 24 hours, in vitro expanded for 5-10 days, and co-cultured with donor NKT cells (HLA-A2−) at a 2:1 (T:NKT) ratio for two days. NKTs are transduced with 1) CAR19.IL2SP-opti15 with scrambled shRNA sequences (Scr), 2) CAR19.IL2SP-opti15 with double knockdown (KD), 3) CAR19.IL2SP-opti15 with double knockout (KO). Panel A of FIG. 28A presents representative flow plots showing total frequency of donor NKT cells on day 0 and day 2 of co-culture (top, FIG. 28B). Absolute cell counts of donor NKT cells are shown in Panel B of FIG. 28A and Panel C of FIG. 28B presents absolute cell counts of recipient T cells on day 2 of co-culture. All data denote mean±s.d., five unique donor-recipient pairs are used. P values are determined using two-way ANOVA with Sidak's correction for multiple comparisons and nonsignificant (P>0.05) values are not shown.

FIGS. 29A and 29B present representative graphs of flow cytometry of transduced NKT cells and absolute cell counts of donor NKT cells and recipient T cells in another aspect. Recipient whole PBMCs (HLA-A2+) are co-cultured with donor NKTs (HLA-A2−) at a 10:1 (PBMC:NKT) ratio for nine days. NKTs are transduced with 1) CAR19.IL2SP-opti15 with scrambled shRNA sequences in place of B2M control (Scr), 2) CAR19.IL2SP-opti15 with double knockdown (KD), 3) CAR19.IL2SP-opti15 with double knockout (KO). Panel A of FIG. 29A presents representative flow plots showing total frequency of donor NKT cells on day 0 and day 9 of co-culture (top, FIG. 29B). Panel B of FIG. 29A shows absolute cell counts of donor NKT cells and Panel C of FIG. 29B shows absolute cell counts of recipient T cells on days 0, 3, 6, and 9 of co-culture. All data denote mean±s.d., three unique donor-recipient pairs are used. P values are determined using two-way ANOVA with Sidak's correction for multiple comparisons and nonsignificant (P>0.05) values are not shown. P values are determined using the two-tailed, paired Student's t-test.

FIG. 30 presents a representative results of in vivo persistence in an in vivo T cell-mediated rejection model in vivo of NKT cells expressing the B2M/CIITA double knockdown construct. Panel A presents the experimental procedure. NSG mice are irradiated at 1.2 Gy on day −1, and on the following day receive 7×10⁶ in vitro expanded human T-cells (day 5-10 post initial OKT3/αCD28 stimulation) from an HLA-A2′ recipient. Four days later, mice receive 2×10⁶ control construct (CAR19.IL2SP-opti15.amiR-SCR-amiR-SCR) or knockdown construct (CAR19.IL2SP-opti15.amiR-b2m-amiR-ciita) transduced NKT cells from an HLA-A2⁺ donor intravenously. RTC=recipient T cells. Panel B presents representative flow plots showing frequencies of donor HLA-A2+ Scr control or double KD NKT cells in peripheral blood on days 6 and 28. Panel C presents the frequency of donor HL-A2+ NKT cells and recipient HLA-A2-T− cells (Panel D) at specified time points. Data denote mean±SD with 7-8 mice per group.

FIG. 31 presents representative results of in vivo persistence in an in vivo PBMC cell-mediated rejection model in vivo of NKT cells expressing the B2M/CIITA double knockdown construct in the presence of allogeneic PBMCs compared to scrambled control NKTs. Panel A presents the experimental procedure. NSG (MHC^{KO}) mice re irradiated at 1.2 Gy on day −1, and then receive intravenously 5×10⁶ freshly isolated PBMC from an HLA-A2⁻ recipient on day 0. Four days later, 5×10⁶ scrambled control or double knockdown transduced NKTs from an HLA-A2⁺ donor are administered intravenously. Panel B presents representative flow plots showing frequencies of donor HLA-A2+ Scr control or double KD NKT cells in peripheral blood on days 6 and 20. Panel C presents the frequency donor HL-A2+ NKT cells and Panel D present the frequency of recipient HLA-A2−T cells at specified time points. Data denote mean±SD with 7-8 mice per group.

FIGS. 32A and 32B present representative results of anti-tumor activity in vivo in the presence of allogeneic T cells compared to scrambled control NKT cells in an In vivo T cell-mediated rejection model with B cell lymphoma xenograft of NKT cells expressing the B2M/CIITA double knockdown construct. Panel A presents the experimental procedure. NSG mice are irradiated at 1.2 Gy and receive intravenously 7×10⁶ in vitro expanded human T cells (days 8-10 postinitial OKT3/αCD28 stimulation) from an HLA-A2-recipient on the following day. One day later, 2×10⁵ firefly luciferase-positive Daudi cells are injected intravenously, followed three days later by 5×10⁶ scrambled control or knockdown transduced NKT cells generated from an HLA-A2+ donor. RTC=recipient T cells. Panel B presents a representative flow plot showing frequencies of donor HLA-A2+ scrambled control (Scr) or double KD NKT cells in peripheral blood of mice on days 6 and 28. Frequencies of HLA-A2+ donor CAR NKT cells (Panel C) and HLA-A2-RTCs in peripheral blood (Panel D) after tumor injection. Panel E presents lymphoma progression measured using IVIS imaging at specified time points. Panel F presents Kaplan-Meier curve showing survival of mice in each experimental group. P values are determined using two-sided log-rank test.

FIG. 33 presents examples of CAR.GPC3.opti-IL15 double knockdown constructs. The constructs comprise sequences encoding either the GPC3-specific scFv from GC33 or the scFv from the humanized YP7.

FIG. 34 presents levels of HLA class I or class II gene knockdown are observed in CAR-GPC3 NKT cells expressing either the humanized GPC3 scFv (YP7) or murine GPC3 scFv (GC33)

FIG. 35 presents expression levels of IL15 in NKT cells expressing humanized GPC3 scFv (YP7) and NKT cells expressing murine GPC3 scFv.

FIG. 36 presents the cytotoxicity levels in cells expressing humanized GPC3 scFv (YP7) and NKT cells expressing murine GPC3 scFv, as measured by the xCelligence assay.

FIG. 37 presents experimental design and the expected anti-tumor activity of CAR.GPC3 NKT cells in an HCC xenograft model.

FIG. 38 presents the expression level of B2M, CIITA, or native IL-15 in CAR.GPC3 NKT cells expressing amiR constructs targeting B2M and CIITA and CAR.GPC3 NKT cells comprising IL15 constructs.

FIG. 39 presents a comparison of IL-15 expression levels in NKT cells expressing constructs having IL-15 coding sequence upstream or downstream of CAR.GPC3.

FIG. 40 presents a heat map illustrating the HLA-specific genes downregulated in G.28BBz.15.miR-expressing NKT cells in comparison with 15G28BBz-expressing NKT cells. Adjusted P value is less than 0.05 and fold change is greater than 2.

FIG. 41 presents a heat map illustrating the HLA-specific and immune effector genes downregulated in YP7.28BBz.15.miR-expressing NKT cells in comparison with 15G28BBz expressing NKT-cells. Adjusted P value is less than 0.05 and fold change is greater than 2.

FIG. 42 presents a heat map illustrating that no significant pathways are enriched in humanized YP7.28BBz.15.miR-expressing NKT cells in comparison with murine G.28BBz.15.miR-expressing NKT cells. Adjusted P value is less than 0.05 and fold change is greater than 2.

Corresponding reference characters indicate corresponding parts throughout the several views. The example(s) set out herein illustrate(s) [one/several] embodiment(s) of the present disclosure but should not be construed as limiting the scope of the present disclosure in any manner.

### DETAILED DESCRIPTION

The present application is directed to methods and compositions related to genetically modified natural killer T cells (NKT cells). NKT cells are a distinct cell type that share some features of both T and NK cells but are distinct from both conventional T cells and also NK cells. NKT cells have divergent development from conventional T cells and NK cells and different functions driven by a unique set of transcriptional regulators. See Kronenberg M, Gapin L. The unconventional lifestyle of NKT cells. Nat. Rev. Immunol. 2002; 2(8):557-568; Godfrey, J C I, 2004, Cohen N R, et al. Shared and distinct transcriptional programs underlie the hybrid nature of iNKT cells. Natdmmunol. 2013; 14(1):90-99). Godfrey et al., identify transcription factors, signal-transduction factors, cell surface molecules, cytokines, and other factors that selectively influence NKT cell development reflecting the unique programming associated with the NKT cell lineage. (Godfrey et al., “Raising the NKT cell family,” Nat. Immunol., 11(3):197-206 (2010) (“Godfrey et al.”) hereby incorporated by reference in its entirety. See also Engel and Kronenberg, “Transcriptional control of the development and function of Vα4i NKT cells,” Current Topics in Microbiology and Immunology, Volume 381, 2014). Many transcription factors and signaling molecules that affect NKT cells differentiation in the thymus do not affect other conventional T cell populations that develop there. As used throughout the present disclosure, the term “T cell” is limited to conventional T cells that are distinguishable from NKT cells. These differences result in different responses to stimuli and genetic changes such as engineered gains and losses of gene expression that make results in non-NKT cells unpredictable.

NKT cells are distinguishable based on whole genome transcription analysis and are equally distant from conventional and NK cell lineages. See Cohen et al. supra. Conventional T cells, also known as T lymphocytes, are an important cell type with the function of fighting pathogens and regulating the immune response. Two hall marks of these cells are expression of an antigen receptor encoded by segments of DNA that rearrange during cell differentiation to form a vast array of receptors. A number of cells fall within this generic definition of a T cell, for example: T helper cells (CD4+ cells) including the sub-types TH1, TH2, TH3, TH17, TFH; cytotoxic T cells (mostly CD8+ cells, also referred to a CTLs); memory T cells (including central memory T cells, effector memory T cells, and resident memory T cells); regulatory T cells, and mucosal associated invariant T cells. Cell surface markers of T cells include the T cell receptor and CD3. Generally T cells do not express CD56 (i.e. are CD56 negative).

NK cells and NKT cells are CD56+. In humans NK cells usually express the cell surface marker CD56, CD161, CD11b, NKp46, NKp44, CD158 and IL-12R. NK cells express a limited repertoire of receptors with an entirely different structure, some of which are also found on NKT cells. Most NK receptors are not highly conserved comparing humans and rodents. NK cells express members of the family of killer-cell-immunoglobulin-like receptors (KIRs), which can be activating or inhibiting, as well as receptors that are members of the lectin (carbohydrate-binding) family of proteins such as NKG2D and CD94NKG2A/C. KIRs are not expressed on NKT cells. NK cells are activated by a number of cell surface receptors, such as KIRs in humans or Ly49 in mice, natural cytotoxic receptors (NCRs), NKG2D and CD94:NKG2 heterodimers. In addition cytokines and chemokines, such as IL-12, IL-15, IL-18, IL-2 and CCLS, play a significant role in NK cell activation.

NKT cells generally can be identified as CD3+CD56+ cells and express a T cell receptor. NKT cells express a T cell receptor and CD3 chains like T cells, but also have markers such CD56 and CD161, like NK cells. Having said that, it is now commonly accepted by experts that they are a distinct lineage of cells. That is they are very different from other T cells and their behavior and properties cannot be predicted from analysis of other T cells, nor are they NK cells. NKT cells are completely different cells to conventional T cells and to NK cells. Due to the unique properties of the NKT cell lineage, observations made with other populations of lymphocytes, such as T cells, NK cells, and B cells, may not predict functional consequences of NKT cell activation.

NKT cells can be identified from other cell types including CD4 T cells, CD8 T cells, regulatory T cells, γδ T cells, B cells, NK cells, monocytes and dendritic cells based on the expression of cell surface markers. See Park et al., “OMIP-069: Forty-Color Full Spectrum Flow Cytometry Panel for Deep Immunophenotyping of Major Cell Subsets in Human Peripheral Blood,” Cytometry Part A 97A:1044-1051 (2020); Hertoghs et al., OMIP-064: A 27-Color Flow Cytometry Panel to Detect and Characterize Human NK Cells and Other Innate Lymphoid Cell Subsets, MAIT Cells, and γδ T Cells, Cytometry Part A 97A:1019-1023 (2020); Sahir et al., Development of a 43 color panel for the characterization of conventional and unconventional T-cell subsets, B cells, NK cells, monocytes, dendritic cells, and innate lymphoid cells using spectral flow cytometry, Cytometry 2020:1-7.

NKT cells are divided into two main types, Type I and Type II. The most significant form of NKT cells, known as type I NKT cells or invariant NKT cells (“iNKT”), have an invariant T cell receptor alpha chain (Vα4i mouse or Vα24i human). Type I NKT (iNKT) cells can be readily detected by the binding of CD1d-based tetramers loaded with αGalCer analogs. The form of the antigen receptor is a limited repertoire due to an invariant alpha chain paired with one of a relatively small number of beta chains. inhibition, or therapeutic use. The antigens recognized by this invariant receptor are glycolipids, for example those found in bacterial cells. The invariant receptor recognizes alpha-galatosylceramide (a-GalCer) a glycolipid originally derived from marine sponges. This compound is similar to microbial glycolipids, and it is now generally assumed to be derived from a microbial symbiont associated with the sponge. NKT cells require antigen presented on a molecule CD1d.

Type II NKT cells also require antigen presentation from CD1d but have a more diverse but still limited TCR repertoire. Type II NKT cells express low levels of the transcription factor PLZF. While Type I NKT cells only recognize α-GalCer, Type II NKT cells recognize sulfatide, lyso-sulfatide, Lyso-PC and Lyso-GL1. Type II NKT cells are more prevalent in humans, but less prevalent in mice. See Dhodpkar and Kumar, “Type II NKT Cells and Their Emerging Role in Health and Disease,” J Immunol. 198(3):1015-1021 (2017).

Two pathways are known for NKT cell activation. NKT cells respond stimulation through their T cell receptor via antigen presented on CD1d molecules. This does not depend upon the involvement of a CD4 or CD8 co-receptor to generate a TCR signal, and the response of these cells is somewhat less dependent on a co-stimulatory signal. In addition, a mechanism for activation of NKT cells exists in the absence of antigen engaging the T cell receptor, via innate inflammatory stimuli, such as IL-12 and IL-18. Once activated T cells are found in the peripheral blood. Similarly NK cells are found in the peripheral blood. In contrast the majority of NKT cells are found in tissues and they migrate away from peripheral blood to the site of tumors, for example as mediated via a two-step process involving CCR2 and CCR6. The mechanisms involved in this migration are specific to NKT cells and not general mechanisms that apply to other lymphocytes.

iNKT cells are readily distinguishable from other T-cell types. See Table 1. Only a small fraction of expanded T cells (a subset of CD4 T cells) can produce tumor-protective Th2 cytokines (IL-4, IL-5, IL-13, IL-10) upon activation either via the T cell receptor (TCR). The majority of T cells (including all CD8+ T cells) and all NK cells produce only anti-tumor Th1 cytokines (i.e. IFN-gamma, GM-CSF, TNF-alpha). In contrast, NKT cells simultaneously produce Th1 and Th2 cytokines.” Depending on the balance of Th1 and Th2 cytokines produced after T cell receptor (TCR) activation, NKT cells can either activate or suppress the immune response. Thus NKT cells have an intriguing paradoxical dual function of immune activation and immune suppression. In contrast other immune cells usually have one primary function, for example fighting pathogens, whilst other subsets of cells are dedicated to regulating the immune response.

TABLE 1
Distinguishing features of iNKT cells

| T CELLS | iNKT CELLS |
|---|---|
| TCR specificity varies | TCR specificity does not vary |
| TCR binds peptides | TCR binds certain glycolipids, for |
| presented on MHC | example natural products and |
| molecules | derivatives from bacterial cell walls, |
| | presented on CD1d TCR/MHC/peptide |
| | complex formed TCR has unique |
| | docking strategy with CD1d |
| Part of the reactive immune | Part of the innate immune system |
| system | |
| Take time to react to a | React very quickly to a ″treat″ |
| ″threat″ | |
| Involved in tissue rejection | Not involved in tissue rejection |
| Tolerant to self-antigens | Can react to self-antigens |
| Non-specifically activated | Can be activated by the cytokines |
| by anti-CD3 agonistic | IL-12 and IL-18 |
| antibody | |
| Primarily located in blood | Generally resident in tissue |
| Do not co-located with | Co-located with tumor associated |
| tumor associated | macrophage in hypoxic tumor |
| macrophages | microenvironment |
| Does not migrate to tumor | Migrates to the tumor microenvironment |
| | via a unique CCR2 and CCR6 mechanism |
| Have a clear hierarchy of | Have mostly effector-memory |
| naïve-central-effector | phenotype when freshly isolated from |
| differentiation | peripheral blood, but can generate |
| | CD62L+ central memory-like cells |
| | upon certain conditions of ex vivo |
| | culture (G. Tian et al.) |

Developmental pathway is distinct for the two cell types
In vitro stimulation/culture of the T cell and NKT cells require different
protocols

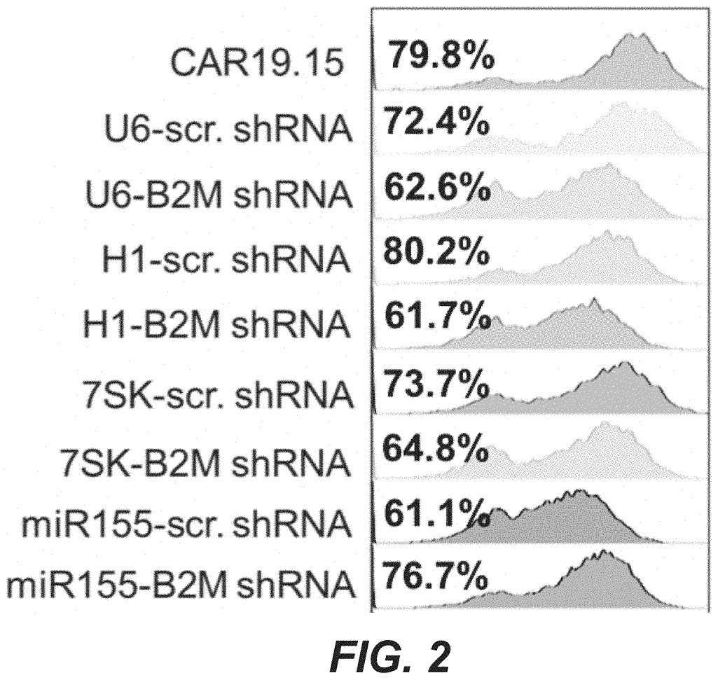

NKT cells also develop in the thymus, however, the positive selection of Type I NKT cells is mediated by CD 1d positive thymocytes. NKT cells are also subject to negative selection by dendritic cells. See Godfrey et al., at FIG. 2 summarizing the development and maturation of T cells and NKT cells in the thymus.

Unless defined otherwise, all technical and scientific terms used herein have the meaning commonly understood by a person skilled in the art to which this invent ion belongs. The following references provide one of skill with a general definition of many of the terms used in this invention: Singleton et al., Dictionary of Microbiology and Molecular Biology (2nd ed. 1994); The Cam bridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein the term “about” refers to plus/minus 10%.

The terms “comprises”, “comprising”, “includes”, “including”, “having” and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a cell” or “at least one cell” may include a plurality of cells, including mixtures thereof.

The terms “comprises”, “comprising”, and are intended to have the broad meaning ascribed to them in U.S. Patent Law and can mean “includes”, “including” and the like.

By “increase” is meant to alter positively by at least 5%. An alteration may be by 5%, 10%, 25%, 30%, 50%, 75%, or even by 100%.

By “decrease” or “reduce” is meant to alter negatively by at least 5%. An alteration may be by 5%, 10%, 25%, 30%, 50%, 75%, or even by 100%.

By “modulate” is meant positively or negatively alter. Exemplary modulations include a 1%, 2%, 5%, 10%, 25%, 50%, 75%, or 100% change.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, a “genetically engineered natural killer T (NKT) cell” or “engineered NKT cell” is an NKT cell that comprises at least one recombinant nucleic acid encoding exogenous protein or a endogenous protein downstream of a non-native promoter. In aspects, genetically engineered NKT cells comprise a recombinant nucleic acid encoding a chimeric antigen receptor.

By “endogenous” is meant a nucleic acid molecule or polypeptide that is normally expressed in a cell or tissue.

By “exogenous” is meant a nucleic acid molecule or polypeptide that is not endogenously present in the cell, or not present at a level sufficient to achieve the functional effects obtained when over-expressed. The term “exogenous” would therefore encompass any recombinant nucleic acid molecule or polypeptide expressed in a cell, such as foreign, heterologous, and over-expressed nucleic acid molecules and polypeptides.

As used herein, the term “artificial microRNAs (amiRNAs)” are molecules that have been developed to promote gene silencing in a similar manner to naturally occurring miRNAs. amiRNAs are generally constructed by replacing the mature miRNA sequence in the pre-miRNA stem-loop with a sequence targeting a gene of interest. These molecules offer a great alternative to silencing approaches that are based on shRNAs and siRNAs because they present the same efficiency as these options and are less cytotoxic. As used herein, the term “embedded” in an artificial microRNA scaffold” refers to the process of replacing a mature miRNA sequence in the pre-miRNA stem-loop with a sequence targeting a gene of interest. In some aspects, the amiR used in the instant disclosure is amiR155. Lagos-Quintana et al., “Identification of tissue-specific microRNAs from mouse.” Curr Biol. 2002 Apr. 30; 12(9):735-9. In another aspect, the amiR used in the instant disclosure is amiR30. Fellmann et al., “An optimized microRNA backbone for effective single-copy RNAi.” Cell Rep. 2013 Dec. 26; 5(6):1704-13. In further aspects, the amiR used in the instant disclosure is an artificial microRNA scaffold known in the art.

A “short hairpin RNA,” “small hairpin RNA” or “shRNA” is an artificial RNA molecule with a tight hairpin turn that can be used to silence target gene expression via RNA interference (RNAi). They typically consist of a stem of 19-29 base pairs (bp), a loop of at least 4 nucleotides (nt), and a dinucleotide overhang at the 3′ end. In some aspects, the term “shRNA” in the instant disclosure may refer to the sense strand or the antisense strand of the “stem” part of a small hairpin RNA. In other aspects, the term “shRNA” may include the sense strand, the antisense strand, and the loop in between.

As used herein, a small hairpin RNA (shRNA) “targeting” a gene of interest refers to an shRNA comprising a sequence of at least 19 contiguous nucleotides that is essentially identical to, or is essentially complementary to, a gene of interest. Aspects of shRNAs functional in this disclosure have sequence complementarity that need not be 100% but is at least sufficient to permit hybridization to RNA transcribed from the target gene to form a duplex under physiological conditions in a cell to permit cleavage by a gene silencing mechanism. Thus, in aspects the segment is designed to be essentially identical to, or essentially complementary to, a sequence of 19 or more contiguous nucleotides in either the target gene or messenger RNA transcribed from the target gene. By “essentially identical” is meant having 100% sequence identity or at least about 80, 81, 82, 83, 84, 85, 86, 87, 88, 89, 90, 91, 92, 93, 94, 95, 96, 97, 98, or 99% sequence identity when compared to the sequence of 19 or more contiguous nucleotides in either the target gene or RNA transcribed from the target gene; by “essentially complementary” is meant having 100% sequence complementarity or at least about 80, 81, 82, 83, 84, 85, 86, 87, 88, 89, 90, 91, 92, 93, 94, 95, 96, 97, 98, or 99% sequence complementarity when compared to the sequence of 19 or more contiguous nucleotides in either the target gene or RNA transcribed from the target gene. In some aspects of this disclosure shRNAs are designed to comprise a sequence having 100% sequence identity with or complementarity to one allele of a given target gene; in other aspects the shRNAs are designed to comprise a sequence having 100% sequence identity with or complementarity to multiple alleles of a given target gene.

Sequence identity is typically measured using sequence analysis software that are widely available in the art. Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

Major histocompatibility complex (MHC) class I and class II proteins play a pivotal role in the adaptive branch of the immune system. Both classes of proteins share the task of presenting peptides on the cell surface for recognition by T cells. Immunogenic peptide-MHC class I (pMHCI) complexes are presented on nucleated cells and are recognized by cytotoxic CD8+ T cells. The presentation of pMHCII by antigen-presenting cells (e.g., dendritic cells (DCs), macrophages, or B cells), on the other hand, can activate CD4+ T cells, leading to the coordination and regulation of effector cells. In all cases, it is a clonotypic T cell receptor that interacts with a given pMHC complex, potentially leading to sustained cell:cell contact formation and T cell activation. Wieczorek et al., “Major Histocompatibility Complex (MEW) Class I and Class II Proteins: Conformational Plasticity in Antigen Presentation.” Frontiers in Immunology, 2017, Mar. 17; 8:292.

Major histocompatibility complex class I and class II share an overall similar fold. The binding platform is composed of two domains, originating from a single heavy α-chain (HC) in the case of MHC class I and from two chains in the case of MHC class II (α-chain and (β-chain). The two domains evolved to form a slightly curved β-sheet as a base and two α-helices on top, which are far enough apart to accommodate a peptide chain in-between. Two membrane-proximal immunoglobulin (Ig) domains support the peptide-binding unit. One Ig domain is present in each chain of MHC class II, while the second Ig-type domain of MHC class I is provided by non-covalent association of the invariant light chain beta-2 microglobulin (B2M) with the HC. Transmembrane helices anchor the HC of MHC class I and both chains of MEW class II in the membrane. Id. Class II transactivator (CIITA) is a transcriptional coactivator that regulates y-interferon-activated transcription of MHC class I and II genes.

The human leukocyte antigen (HLA) system or complex is a group of related proteins that are encoded by the MEW gene complex in humans. These cell-surface proteins are responsible for the regulation of the immune system.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. The cells disclosed herein can be autologous cells, syngeneic cells, allogenic cells and even in some cases, xenogeneic cells.

By “isolated cell” is meant a cell that is separated from the molecular and/or cellular components that naturally accompany the cell.

The term “chimeric antigen receptor” or “CAR,” as used herein, refers to an artificial T cell receptor that is engineered to be expressed on an immune effector cell and specifically bind an antigen. In aspects, CARs comprise and ectodomain, a transmembrane domain, and an endodomain. In certain aspects, a CAR can comprise an ectodomain and transmembrane domain without an endodomain, but more CARs of the present application include the endodomain and provide for intracellular signaling.

By “receptor” is meant a polypeptide, or portion thereof, present on a cell membrane that selectively binds one or more ligands.

As used herein, an “antigen recognition domain” generally comprises a single chain variable fragment (scFv) specific for a particular cancer antigen. In some aspects, where there are two or more CARs in the same cell, the second CAR may comprise an scFv specific for another particular antigen.

As used herein, the term “single-chain variable fragment” or “scFv” is a fusion protein of the variable regions of the heavy (VH) and light chains (VL) of an immunoglobulin covalently linked to form a VH:: VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

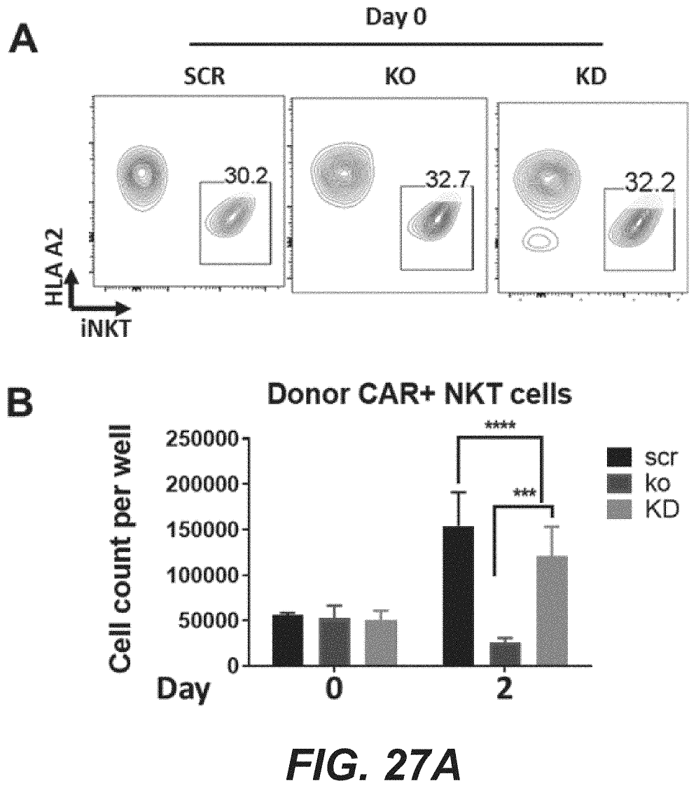

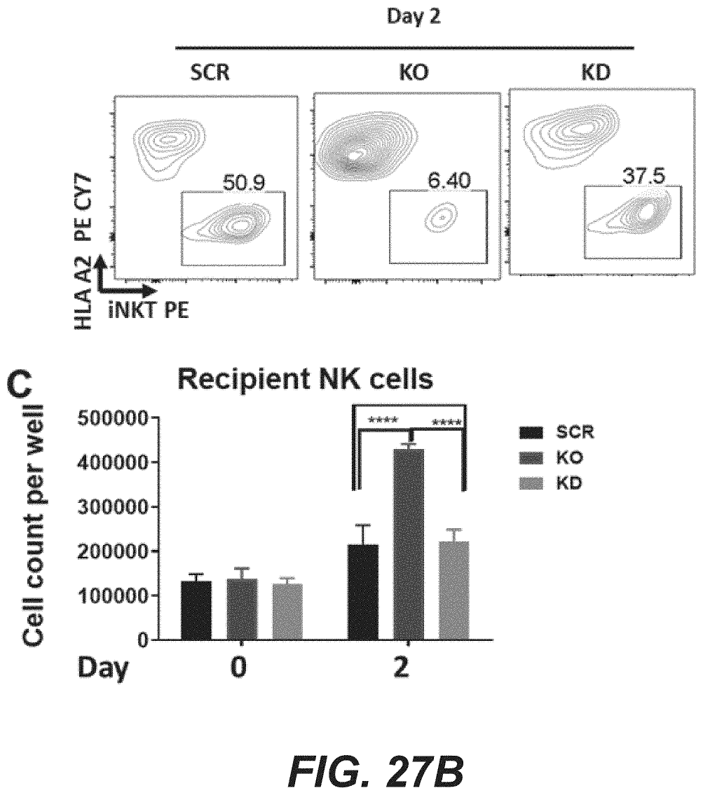

As used herein, a “transmembrane domain” is a region of predominantly of nonpolar amino acid residues that when the protein is expressed, traverses the bilayer at least once. Generally, the transmembrane domain is encoded by 18 to 21 amino acid residues and adopts an alpha helical configuration. As used herein, the transmembrane domain may be of any kind known in the art. In aspects the transmembrane domain is although in some cases it is CD28. Other sources include CD3-C, CD4, or CD8. An exemplary combination of an ectodomain is shown in FIG. 27b of PCT/US2022/015525. Other suitable transmembrane regions can be obtained from CD16, NKp44, NKp46, and NKG2d.

As used herein, the term “endodomain” refers to the intracellular domain of a CAR that provides for signal transmission in a cell. Generally, the endodomain can be further divided into two parts, a stimulatory domain and optionally, a co-stimulatory domain. The co-stimulatory domain is shown to be arranged amino-terminal to the stimulatory in FIG. 27a of PCT/US2022/015525, but the present specification also provides for an amino terminal stimulatory domain and followed by a co-stimulatory domain when present. The most commonly used endodomain component is CD3-zeta that contains 3 ITAMs and that transits an activation signal to the NKT cell after the antigen is bound. Other suitable stimulatory domains can be obtained from 2B4 (CD244), TNF receptor superfamily member 9 (Gene ID 3604, e.g., 4-1BB or CD137), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

As used herein, the term “ectodomain” refers to the extracellular portion of a CAR and encompasses a signal peptide, an antigen recognition domain, and a spacer or hinge region that links the antigen recognition domain to the transmembrane domain. When expressed, the signal peptide may be removed.

The term “tumor antigen” as used herein refers to an antigen (e.g., a polypeptide, glycoprotein, or glycolipid) that is uniquely or differentially expressed on a tumor cell compared to a normal or non-neoplastic cell. With reference to the invention, a tumor antigen includes any polypeptide expressed by a tumor that is capable of being recognized by an antigen recognizing receptor (e.g., CD19, Muc-1) or capable of suppressing an immune response via receptor-ligand binding (e.g., CD47, PD-L1/L2, 87.112).

By “tissue antigen” is meant an antigen (e.g., a polypeptide or glycoprotein or glycolipid) that is uniquely or differentially expressed on a normal or non-neoplastic cell or tissue compared to a tumor cell.

The terms “subject,” “individual,” and “patient,” are used interchangeably herein and refer to any vertebrate subject, including, without limitation, mammals, preferably a humans and other primates, including non-human primates such as laboratory animals including rodents such as mice, rats and guinea pigs; The term does not denote a particular age. Thus, both adult and newborn individuals are intended to be covered.

By “effective amount” is meant an amount sufficient to have a therapeutic effect. In one embodiment, an “effective amount” is an amount sufficient to arrest, ameliorate, or inhibit the continued proliferation, growth, or metastasis (e.g., invasion, or migration) of a neoplasia.

By a “heterologous nucleic acid molecule or polypeptide” is meant a nucleic acid molecule (e.g., acDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “immunoresponsive cell” is meant a cell that functions in an immune response or a progenitor, or progeny thereof.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “obtaining” as in “obtaining the agent” is intended to include purchasing, synthesizing or otherwise acquiring the agent (or indicated substance or material).

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells). Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

By “operably linked”, as used herein, is meant the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

By “promoter” is meant a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence.

By “reference” or “control” is meant a standard of comparison. For example, the immune response of a cell expressing a CAR and an additional protein may be compared to the immune response of a corresponding non-engineered cell expressing CAR alone.

By “analog” is meant a structurally related polypeptide or nucleic acid molecule having the function of a reference polypeptide or nucleic acid molecule.

By “disease” is meant any condition or disorder that damages or interferes with the normal function of a cell, tissue, or organ. Examples of diseases include neoplasia or pathogen infection of cell.

As used herein, the term “engineering” refers to the genetic modification of a cell to introduce one or more exogenous nucleic acid sequences. Preferably, engineering introduced exogenous nucleic acid sequences that are transcribed and translated to express a protein. Introducing exogenous nucleic acid sequences can be performed using methods known in the art including transformation, transfection and transduction.

The present disclosure provides for, and includes, a recombinant construct for suppressing the expression of an endogenous major histocompatibility complex (MHC) gene, comprising a DNA sequence encoding a chimeric antigen receptor (CAR) recognizing a tumor antigen and a DNA sequence encoding a small hairpin RNA (shRNA) sequence targeting an MHC class I or MHC class II gene, where the shRNA sequence is embedded in an artificial microRNA (amiR) scaffold.

In one aspect, the recombinant construct as disclosed herein further comprises a DNA sequence encoding a cytokine. In some aspects, the cytokine is interleukin-15 (IL-15), IL-7, IL-12, IL-18, IL-21, IL-27, IL-33, or a combination thereof. In one aspect, the cytokine is IL-15. In one aspect, the IL-15 is a human IL-15. In one aspect, the DNA sequence encoding an IL-15 is codon-optimized. In another aspect, the IL15 comprises an IL-2 signal peptide. In one aspect, the DNA sequence encoding an IL-15 in conjunction with IL15Ra. In another aspect, the DNA sequence encoding an IL-15 in conjunction with the IL15Ra Sushi domain. In some aspects, the DNA sequence encoding an IL-15 is upstream of the DNA sequence encoding a CAR. In other aspects, the DNA sequence encoding an IL-15 is downstream of the DNA sequence encoding a CAR.

In some aspects, the amiR used in the instant disclosure is amiR155. In another aspect, the amiR used in the instant disclosure is amiR30. In further aspects, the amiR used in the instant disclosure is an artificial microRNA scaffold known in the art.

In some aspect, the MHC class I and class II genes are human leukocyte antigen (HLA) class I and class II genes.

In some aspects, the MHC class I gene encodes a β2-microglobulin (B2M).

In some aspects, the MHC class II gene encodes an invariant chain (Ii) or a class II transactivator (CIITA).

In some aspects, the recombinant constructs as disclosed herein comprise a first shRNA sequence embedded in a first amiR scaffold and a second shRNA sequence embedded in a second amiR scaffold. In some aspects, the first shRNA sequence targets a MHC class I gene and the second shRNA sequence targets a MEW class I gene. In one aspect, the first amiR scaffold and the second amiR scaffold are from the same amiR sequence. In other aspects, the first amiR scaffold and the second amiR scaffold are from different amiR sequences.

In some aspects, the recombinant constructs as disclosed herein are suitable for expression in different types of immune cells. In certain other embodiments, the tumor antigen-specific CARs described herein are expressed in different types of immune cells. Examples of immune cells include, but are not limited to, T cells, NK cells, dendritic cells, NKT cells, MAΓΓ cells, γδ-T cells, or a mixture thereof. The T cells may be CD4+ T cells, CD8+ T cells, or Treg cells, Th1 T cells, Th2 T cells, Th17 T cells, unspecific T cells, or a population of T cells that comprises a combination of any of the foregoing. The immune cells may harbor a polynucleotide that encodes the CAR, and the polynucleotide may further comprise a suicide gene.

The present disclosure also provides for, and includes, a method for limiting rejection of an engineered natural killer T (NKT) cell by the immune system of an allogeneic host, comprising transducing an NKT cell with the recombinant constructs disclosed herein, where the expression of the endogenous MHC gene in the NKT cell is suppressed by the shRNA.

In some aspects, the present disclosure also provides for, and includes, a method for limiting rejection of an engineered immune cell by the immune system of an allogeneic host, comprising transducing an immune cell with the recombinant constructs disclosed herein, where the expression of the endogenous MHC gene in the immune cell is suppressed by the shRNA.

In some aspect, the immune system of an allogeneic host comprise immune cells including, but are not limited to, T cells, NK cells, dendritic cells, NKT cells, MAΓΓ cells, γδ-T cells, or a mixture thereof. The T cells may be CD4+ T cells, CD8+ T cells, or Treg cells, Th1 T cells, Th2 T cells, Th17 T cells, unspecific T cells, or a population of T cells that comprises a combination of any of the foregoing.

In some aspects, the expression level of the endogenous MHC gene in the engineered immune cell is decreased by at least 10%, at least 15%, at least 20%, at least 25%, at least 30%, at least 35%, as least 40%, as least 45%, at least 50%, at least 55%, at least 60%, at least 65%, at least 70%, at least 75%, at least 80%, at least 85%, at least 90%, or at least 95% at 2 days post-transduction. In some aspects, the expression level of the endogenous MHC gene in the engineered immune cell is decreased by 10% to 15%, 10% to 20%, 10% to 25%, 10% to 30%, 10% to 35%, 10% to 40%, 10 to 45%, 10% to 50%, 10% to 55%, 10% to 60%, 10% to 65%, 10% to 70%, 10% to 75%, 10% to 80%, 10% to 85%, 10% to 90%, 10% to 95%, 15% to 20%, 15% to 25%, 15% to 30%, 15% to 35%, 15% to 40%, 15% to 45%, 15% to 50%, 15% to 55%, 15% to 60%, 15% to 65%, 15% to 70%, 15% to 75%, 15% to 80%, 15% to 85%, 15% to 90%, 15% to 95%, 20% to 25%, 20% to 30%, 20% to 35%, 20% to 40%, 20% to 45%, 20% to 50%, 20% to 55%, 20% to 60%, 20% to 65%, 20% to 70%, 20% to 75%, 20% to 80%, 20% to 85%, 20% to 90%, 20% to 95%, 25% to 30%, 25% to 35%, 25% to 40%, 25% to 45%, 25% to 50%, 25% to 55%, 25% to 60%, 25% to 65%, 25% to 70%, 25% to 75%, 25% to 80%, 25% to 85%, 25% to 90%, 25% to 95%, 30% to 35%, 30% to 40%, 30% to 45%, 30% to 50%, 30% to 55%, 30% to 60%, 30% to 65%, 30% to 70%, 30% to 75%, 30% to 80%, 30% to 85%, 30% to 90%, 30% to 95%, 35% to 40%, 35% to 45%, 35% to 50%, 35% to 55%, 35% to 60%, 35% to 65%, 35% to 70%, 35% to 75%, 35% to 80%, 35% to 85%, 35% to 90%, 35% to 95%, 40% to 45%, 40% to 50%, 40% to 55%, 40% to 60%, 40% to 65%, 40% to 70%, 40% to 75%, 40% to 80%, 40% to 85%, 40% to 90%, 40% to 95%, 45% to 50%, 45% to 55%, 45% to 60%, 45% to 65%, 45% to 70%, 45% to 75%, 45% to 80%, 45% to 85%, 45% to 90%, 45% to 95%, 50% to 55%, 50% to 60%, 50% to 65%, 50% to 70%, 50% to 75%, 50% to 80%, 50% to 85%, 50% to 90%, 50% to 95%, 55% to 60%, 55% to 65%, 55% to 70%, 55% to 75%, 55% to 80%, 55% to 85%, 55% to 90%, 55% to 95%, 60% to 65%, 60% to 70%, 60% to 75%, 60% to 80%, 60% to 85%, 60% to 90%, 60% to 95%, 65% to 70%, 65% to 75%, 65% to 80%, 65% to 85%, 65% to 90%, 65% to 95%, 70% to 75%, 70% to 80%, 70% to 85%, 70% to 90%, 70% to 95%, 75% to 80%, 75% to 85%, 75% to 90%, 75% to 95%, 80% to 85%, 80% to 90%, 80% to 95%, 85% to 90%, 85% to 95%, or 90% to 95% at 2 days post-transduction.

In some aspects, the expression level of the endogenous MHC gene in the engineered immune cell is decreased by at least 10%, at least 15%, at least 20%, at least 25%, at least 30%, at least 35%, as least 40%, as least 45%, at least 50%, at least 55%, at least 60%, at least 65%, at least 70%, at least 75%, at least 80%, at least 85%, at least 90%, or at least 95% at 7 days post-transduction. In some aspects, the expression level of the endogenous MHC gene in the engineered immune cell is decreased by 10% to 15%, 10% to 20%, 10% to 25%, 10% to 30%, 10% to 35%, 10% to 40%, 10 to 45%, 10% to 50%, 10% to 55%, 10% to 60%, 10% to 65%, 10% to 70%, 10% to 75%, 10% to 80%, 10% to 85%, 10% to 90%, 10% to 95%, 15% to 20%, 15% to 25%, 15% to 30%, 15% to 35%, 15% to 40%, 15% to 45%, 15% to 50%, 15% to 55%, 15% to 60%, 15% to 65%, 15% to 70%, 15% to 75%, 15% to 80%, 15% to 85%, 15% to 90%, 15% to 95%, 20% to 25%, 20% to 30%, 20% to 35%, 20% to 40%, 20% to 45%, 20% to 50%, 20% to 55%, 20% to 60%, 20% to 65%, 20% to 70%, 20% to 75%, 20% to 80%, 20% to 85%, 20% to 90%, 20% to 95%, 25% to 30%, 25% to 35%, 25% to 40%, 25% to 45%, 25% to 50%, 25% to 55%, 25% to 60%, 25% to 65%, 25% to 70%, 25% to 75%, 25% to 80%, 25% to 85%, 25% to 90%, 25% to 95%, 30% to 35%, 30% to 40%, 30% to 45%, 30% to 50%, 30% to 55%, 30% to 60%, 30% to 65%, 30% to 70%, 30% to 75%, 30% to 80%, 30% to 85%, 30% to 90%, 30% to 95%, 35% to 40%, 35% to 45%, 35% to 50%, 35% to 55%, 35% to 60%, 35% to 65%, 35% to 70%, 35% to 75%, 35% to 80%, 35% to 85%, 35% to 90%, 35% to 95%, 40% to 45%, 40% to 50%, 40% to 55%, 40% to 60%, 40% to 65%, 40% to 70%, 40% to 75%, 40% to 80%, 40% to 85%, 40% to 90%, 40% to 95%, 45% to 50%, 45% to 55%, 45% to 60%, 45% to 65%, 45% to 70%, 45% to 75%, 45% to 80%, 45% to 85%, 45% to 90%, 45% to 95%, 50% to 55%, 50% to 60%, 50% to 65%, 50% to 70%, 50% to 75%, 50% to 80%, 50% to 85%, 50% to 90%, 50% to 95%, 55% to 60%, 55% to 65%, 55% to 70%, 55% to 75%, 55% to 80%, 55% to 85%, 55% to 90%, 55% to 95%, 60% to 65%, 60% to 70%, 60% to 75%, 60% to 80%, 60% to 85%, 60% to 90%, 60% to 95%, 65% to 70%, 65% to 75%, 65% to 80%, 65% to 85%, 65% to 90%, 65% to 95%, 70% to 75%, 70% to 80%, 70% to 85%, 70% to 90%, 70% to 95%, 75% to 80%, 75% to 85%, 75% to 90%, 75% to 95%, 80% to 85%, 80% to 90%, 80% to 95%, 85% to 90%, 85% to 95%, or 90% to 95% at 7 days post-transduction.

In some aspects, the expression level of the endogenous MHC gene is decreased by at least 10%, at least 15%, at least 20%, at least 25%, at least 30%, at least 35%, as least 40%, as least 45%, at least 50%, at least 55%, at least 60%, at least 65%, at least 70%, at least 75%, at least 80%, at least 85%, at least 90%, or at least 95% at 14 days post-transduction. In some aspects, the expression level of the endogenous MHC gene in the engineered immune cell is decreased by 10% to 15%, 10% to 20%, 10% to 25%, 10% to 30%, 10% to 35%, 10% to 40%, 10 to 45%, 10% to 50%, 10% to 55%, 10% to 60%, 10% to 65%, 10% to 70%, 10% to 75%, 10% to 80%, 10% to 85%, 10% to 90%, 10% to 95%, 15% to 20%, 15% to 25%, 15% to 30%, 15% to 35%, 15% to 40%, 15% to 45%, 15% to 50%, 15% to 55%, 15% to 60%, 15% to 65%, 15% to 70%, 15% to 75%, 15% to 80%, 15% to 85%, 15% to 90%, 15% to 95%, 20% to 25%, 20% to 30%, 20% to 35%, 20% to 40%, 20% to 45%, 20% to 50%, 20% to 55%, 20% to 60%, 20% to 65%, 20% to 70%, 20% to 75%, 20% to 80%, 20% to 85%, 20% to 90%, 20% to 95%, 25% to 30%, 25% to 35%, 25% to 40%, 25% to 45%, 25% to 50%, 25% to 55%, 25% to 60%, 25% to 65%, 25% to 70%, 25% to 75%, 25% to 80%, 25% to 85%, 25% to 90%, 25% to 95%, 30% to 35%, 30% to 40%, 30% to 45%, 30% to 50%, 30% to 55%, 30% to 60%, 30% to 65%, 30% to 70%, 30% to 75%, 30% to 80%, 30% to 85%, 30% to 90%, 30% to 95%, 35% to 40%, 35% to 45%, 35% to 50%, 35% to 55%, 35% to 60%, 35% to 65%, 35% to 70%, 35% to 75%, 35% to 80%, 35% to 85%, 35% to 90%, 35% to 95%, 40% to 45%, 40% to 50%, 40% to 55%, 40% to 60%, 40% to 65%, 40% to 70%, 40% to 75%, 40% to 80%, 40% to 85%, 40% to 90%, 40% to 95%, 45% to 50%, 45% to 55%, 45% to 60%, 45% to 65%, 45% to 70%, 45% to 75%, 45% to 80%, 45% to 85%, 45% to 90%, 45% to 95%, 50% to 55%, 50% to 60%, 50% to 65%, 50% to 70%, 50% to 75%, 50% to 80%, 50% to 85%, 50% to 90%, 50% to 95%, 55% to 60%, 55% to 65%, 55% to 70%, 55% to 75%, 55% to 80%, 55% to 85%, 55% to 90%, 55% to 95%, 60% to 65%, 60% to 70%, 60% to 75%, 60% to 80%, 60% to 85%, 60% to 90%, 60% to 95%, 65% to 70%, 65% to 75%, 65% to 80%, 65% to 85%, 65% to 90%, 65% to 95%, 70% to 75%, 70% to 80%, 70% to 85%, 70% to 90%, 70% to 95%, 75% to 80%, 75% to 85%, 75% to 90%, 75% to 95%, 80% to 85%, 80% to 90%, 80% to 95%, 85% to 90%, 85% to 95%, or 90% to 95% at 14 days post-transduction.

In some aspects, the NKT cell is a CD1d-restrictive NKT cell.

The present disclosure further provides for, and includes, an engineered NKT cell transduced with the recombinant constructs as disclosed herein, or produced by a method disclosed herein, where the expression of the endogenous MHC gene in the NKT cell is significantly suppressed compared with a control NKT cell not transduced with the recombinant construct.

The present disclosure also provides for, and includes, an engineered immune cell transduced with the recombinant constructs as disclosed herein, or produced by a method disclosed herein, where the expression of the endogenous MHC gene in the immune cell is significantly suppressed compared with a control immune cell not transduced with the recombinant construct. Examples of immune cells include, but are not limited to, T cells, NK cells, dendritic cells, NKT cells, MAΓΓ cells, γδ-T cells, or a mixture thereof. The T cells may be CD4+ T cells, CD8+ T cells, or Treg cells, Th1 T cells, Th2 T cells, Th17 T cells, unspecific T cells, or a population of T cells that comprises a combination of any of the foregoing.

In some aspects, the engineered NKT cell has improved resistance to rejection by allogeneic T cells or PBMCs.

In some aspects, the engineered NKT cell has improved resistance to destruction by allogeneic natural killer cells.

As provided herein, in aspects, a genetically engineered NKT cell is a Type I NKT cell. In an aspect the Type I NKT cell is a CD62L positive (CD62L+) NKT cell. Generally, the NKT cells of the present disclosure are isolated from human peripheral blood and have undergone less than 20 days of culture prior to introducing a gene construct to produce a genetically engineered NKT cell.

In aspects, the genetically engineered NKT cell of the present disclosure are further characterized by the expression of the cell markers CD4, CD28, 4-1BB, CD45RO (Gene ID5788), OX40, CCR7, and combinations thereof. The expression of these markers is closely associated with trafficking of the NKT cells to the tumor site where they can mediate anti-tumor responses. In further aspects, the genetically engineered NKT cells express markers of NKT cell survival and memory such as, but not limited to, S1PR1, IL-7Ra, IL21R. In aspects, the genetically engineered NKT cells of the present disclosure express low levels of the exhaustion markers TIM-3, LAG3, and PD-1.

The present disclosure provides for and includes CAR proteins that comprise antibody recognition domains that recognize a cancer antigen. In aspects, the CAR comprises an antibody recognition domain for a cancer antigen, a spacer or hinge region, a transmembrane domain, and an endodomain. In an aspect, the antibody recognition domain is a single-chain variable fragment (scFv). In certain aspects the antibody recognition domain is directed at cancer antigens on the cell surface of cancer cells that express an antigen of interest, for example. In aspects, the endodomain includes a stimulatory domain, such as those derived from the T cell receptor z-chain. In other aspect, the stimulatory domains of the present specification include, but are not limited to, endodomains from co-stimulatory molecules such as CD27, CD28, 4-IBB, and OX40 or the signaling components of cytokine receptors such as IL7 and IL15. In aspects, co-stimulatory molecules are employed to enhance the activation, proliferation, and cytotoxicity of the NKT cells produced by the CAR after antigen engagement. In specific aspects, the co-stimulatory molecules are CD28, OX40, or 4-1BB.

Included, and provided by the present disclosure are cancer antigens such as Melanoma-associated antigen (MAGE), Preferentially expressed antigen of melanoma (PRAME), CD19, CD20, CD22, K-light chain, CD30, CD33, CD123, CD38, CD138, ROR1, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-a, CD44v6, CD44v7/8, 8H9, NCAM, VEGF receptors, 5T4, Fetal AchR, or CD44v6. In an aspect, the cancer antigen is selected from the group consisting of CD19, GD2, and glypican-3 (GPC3). In another aspect, the cancer antigen is CD19. In an aspect, the cancer antigen is GD2. In yet another aspect, the cancer antigen is GPC3.

Also included and provided for by the present disclosure are genetically engineered NKT cells comprising two or more CAR molecules that recognize cancer antigens selected from the group consisting of MAGE, PRAME, CD19, CD20, CD22, K-light chain, CD30, CD33, CD123, CD38, CD138, ROR1, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-a, CD44v6, CD44v7/8, 8H9, NCAM, VEGF receptors, 5T4, Fetal AchR, and CD44v6.

In certain aspect, the antigen recognition domain comprises a single-chain variable fragment (scFv). In certain aspect, the antigen recognition domain recognizes a cancer antigen on the cell surface of cancel cells. Non-limiting examples of cancer antigens include any one of Melanoma-associated antigen (MAGE), Preferentially expressed antigen of melanoma (PRAME), CD19, CD20, CD22, K-light chain, CD30, CD33, CD123, CD38, CD138, ROR1,ErbB2,ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-a, CD44v6, CD44v7/8, 8H9, NCAM, VEGF receptors, 5T4, Fetal AchR, NKG2D ligands, or CD44v6. In some cases, the antigen recognition domain recognizes CD19, CD22, CD30, GD2, GPC3, CSPG4, HER2, CEA, or Mesothelin. In one particular aspect, the antigen recognition domain comprises a single-chain variable fragment (scFv) from the CD19-specific antibody FMC-63. In another particular aspect, the antigen recognition domain comprises a single-chain variable fragment (scFv) from the GD2-specific antibody 14G2a. In another particular aspect, the antigen recognition domain comprises a single-chain variable fragment (scFv) from the GPC3-specific antibody GC33 or YP7.

In one aspect, the endodomain sequence in the expression construct according to the present disclosure comprises a cytoplasmic signaling domain, such as those derived from the T cell receptor ζ-chain, in order to produce stimulatory signals for NKT cell proliferation and effector function following engagement of the antigen recognition domain with the target antigen. Non-limiting examples of the endodomain sequences include endodomains from co-stimulatory molecules such as CD27, CD28, 4-IBB, and OX40 or the signaling components of cytokine receptors such as IL7 and IL15. In certain aspects, co-stimulatory molecules are employed to enhance the activation, proliferation, and cytotoxicity of the NKT cells after antigen engagement. In specific aspects, the co-stimulatory molecules are CD28, OX40, and 4-1BB. In one aspect, the endodomain of the CAR according to the present disclosure is utilized for signal transmission in the cell after antigen recognition and cluster of the receptors. In one aspect, the endodomain comprises a CD3-zeta that contains 3 ITAMs and that transmits an activation signal to the NKT cell after the antigen is bound. In certain aspects, additional co-stimulatory signaling is utilized, such as CD3-zeta in combination with CD28, 4-IBB, and/or OX40. In one particular aspect, the endodomain sequence comprises the signal sequence of 4-1BB fused in-frame to a CD3-zeta chain.

The transmembrane domain may be of any kind. In one aspect, the transmembrane domain comprises the transmembrane domain of CD28. In another aspect, the transmembrane domain comprises the transmembrane domain of CD8.

In one particular aspect, the CAR.CD19, CAR.GD2, and CAR.GPC3 constructs are made as previously described (Heczey et al., 2014; Pule et al., A chimeric T cell antigen receptor that augments cytokine release and supports clonal expansion of primary human T cells.

Mol. Ther. 2005; 12(5):933-941) and contain a scFv from the CD19-specific antibody FMC-63 or the GD2-specific antibody 14G2a or the GPC3-specific antibody GC33, or YP7, connected via a short spacer derived from the IgG1 hinge region to the transmembrane domain derived from CD8a, followed by signaling endodomain sequences of 4-1BB fused with ζ chain.

Expression constructs according to the present disclosure can be introduced into the cells as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. The constructs once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In particular aspects, there are methods of generating cells encompassed by the disclosure, including cells that have downregulation of B2M, CIITA, or both. Such cells also may express one or more types of engineered receptors.

In some aspects, the method of producing the cells includes the step of obtaining cells to be manipulated, although in other cases the obtaining step is not included in the method. The donor cells may be obtained from a healthy subject, including one that does not have cancer, for example. The cells may or may not be expanded prior to recombinant manipulation to downregulate B2M and/or CIITA. In some methods, the cells may be selected to express or lack expression of a marker, for example whereupon such selection allows for enhanced expansion of the cells. For example, part of the method of producing the cells may include steps for selecting for expression of CD62L, expression of CD4, and/or reduced or absent expression of PD1.

In particular embodiments, cells of the disclosure are manipulated to express an entity other than the agent that downregulates B2M and/or CIITA, and the entity may be an engineered receptor, a cytokine, or another gene product. In specific embodiments, the entity is a chimeric antigen receptor (CAR). In some cases, the step that renders the cell to downregulate B2M and/or CIITA is a concomitant step that renders the cells capable of expressing the other entity, although in alternative cases these are different steps. In specific embodiments, when the cells are simultaneously engineered to downregulate B2M and/or CIITA and to express a CAR, it is because the agent that downregulates B2M and/or CIITA and the CAR are expressed on the same vector. However, in other cases the agent that downregulates B2M and/or CIITA and the CAR are expressed from different vectors.

Methods of the disclosure may or may not include steps of generating vectors to be introduced to the donor cells or expanded progeny thereof. Production of recombinant vectors is well-known in the art, and a variety of vectors may be utilized, including viral or non-viral vectors. In cases where a single vector encompasses both an agent that downregulates B2M and/or CIITA and an engineered receptor such as a CAR, the skilled artisan recognizes that design of the vector will take size constraints (for example) for the cells into consideration.

In cases wherein the cells to be manipulated are T cells, the endogenous T cell receptor of the cells may be downregulated or knocked out, such as using routine methods in the art.

Aspects of the disclosure include a cell or cells encompassed by the disclosure for use in the treatment of a medical condition, such as cancer or a premalignant condition, in a subject. The cells may be used for any type of cancer, including neuroblastoma, breast cancer, cervical cancer, ovary cancer, endometrial cancer, melanoma, bladder cancer, lung cancer, pancreatic cancer, colon cancer, prostate cancer, hematopoietic tumors of lymphoid lineage, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, myeloid leukemia, acute myelogenous leukemia (AML), chronic myelogenous leukemia, thyroid cancer, thyroid follicular cancer, tumors of mesenchymal origin, fibrosarcoma, rhabdomyosarcomas, melanoma, uveal melanoma, teratocarcinoma, neuroblastoma, glioma, glioblastoma, benign tumor of the skin, renal cancer, anaplastic large-cell lymphoma, esophageal squamous cells carcinoma, hepatocellular carcinoma (HCC), follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), or a hereditary cancer syndrome selected from Li-Fraumeni syndrome and Von Hippel-Lindau syndrome (VHL). In specific embodiments, the premalignant condition is myelodysplastic syndrome (MDS).

In particular aspects of the disclosure there are methods of treating a disease with cells encompassed in the disclosure. Although the disease may be of any kind, in specific embodiments the disease is cancer. Any type of cancer may be treated, including neuroblastoma, breast cancer, cervical cancer, ovary cancer, endometrial cancer, melanoma, bladder cancer, lung cancer, pancreatic cancer, colon cancer, prostate cancer, hematopoietic tumors of lymphoid lineage, leukemia, acute lymphocytic leukemia, chronic lymphocytic leukemia, B-cell lymphoma, Burkitt's lymphoma, multiple myeloma, Hodgkin's lymphoma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), tumors of mesenchymal origin, fibrosarcoma, rhabdomyosarcomas, melanoma, uveal melanoma, teratocarcinoma, neuroblastoma, glioma, glioblastoma, benign tumor of the skin, renal cancer, anaplastic large-cell lymphoma, esophageal squamous cells carcinoma, hepatocellular carcinoma, follicular dendritic cell carcinoma, intestinal cancer, muscle-invasive cancer, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, prostatic hypertrophy, myelodysplasia, Waldenstrom's macroglobinaemia, nasopharyngeal, neuroendocrine cancer myelodysplastic syndrome, mesothelioma, angiosarcoma, Kaposi's sarcoma, carcinoid, oesophagogastric, fallopian tube cancer, peritoneal cancer, papillary serous mullerian cancer, malignant ascites, gastrointestinal stromal tumor (GIST), or a hereditary cancer syndrome selected from Li-Fraumeni syndrome and Von Hippel-Lindau syndrome (VHL). In specific embodiments, the disease is myelodysplastic syndrome (MDS).

An effective amount of cells of the disclosure having reduced expression of B2M, CIITA, or both, are provided to a subject in need of therapy with the cells. The amount may be of any quantity as long as at least one symptom of the disease is ameliorated. In specific embodiments, the cells are provided in a range of at least from about 1×1O6 to about 1×1O9 cells, even more desirably, from about 1×1O7 to about 1×1O9 cells, although any suitable amount can be utilized either above, e.g., greater than 1×1O9 cells, or below, e.g., less than 1×1O7 cells. In specific embodiments, one or more doses of the cells are provided to the subject, and subsequent doses may be separated on the order of minutes, hours, days, weeks, months or years. In some cases, separate deliveries of the cells have different amounts of cells. For example, an initial dose of the cells may be greater or lower than one or more subsequent doses.

The individual being treated may be an adult, adolescent, child, infant or animal. The individual may be a mammal, including a human, dog, cat, horse, cow, sheep, pig, and so forth. The individual may be of any gender, race, genetic background, and so forth. The individual may or may not have a personal and/or family history of cancer. The cells to be manipulated for downregulation of expression of B2M and/or CIITA may or may not be obtained from a family member. In cases wherein the individual has cancer, the cancer may be of any stage or grade, and the cancer may be primary, metastatic, recurrent, sensitive, refractory, and so forth.

In some aspects, one or more therapies in addition to the immunotherapy of the disclosure may be provided to the subject, such as surgery, radiation, hormone therapy, another, nonidentical immunotherapy, chemotherapy, or a combination thereof.

In some aspects, the cells are employed for prevention of cancer in a subject, including, for example, a subject with a personal and/or family history of cancer.

Cells may be delivered to the subject in any suitable manner, including by injection, for example. It is in particular envisaged that the cells are administered to the subject via infusion or injection. Administration of the suitable compositions may be effected by different ways, e.g., by intravenous, subcutaneous, intraperitoneal, intramuscular, topical, parenteral, transdermal, intraluminal, intra-arterial, intrathecal or intradermal administration. The cells may be provided by direct injection into a cancer. Administration of the cells may be systemic or local.

The cells may or may not be targeted to a hypoxic environment associated with the cancer. In such cases, any regulatory element(s) to effect expression from an expression construct(s) in the cell may be effective in hypoxic environments.

In some aspects, compositions comprising allogeneic NKTs as described herein for use in the treatment of a medical condition, such as cancer or a premalignant condition in an individual are provided. Such compositions are off-the shelf products which can be administered to any individual, regardless whether the HLA matches or not. Such composition has significant advantages for patients with regards to immediate availability, safety and therapeutic potential. Further to the cells described herein, said compositions may comprise, without being limited to, suspending agents, anti-oxidants, buffers, bacteriostats and solutes.

Any of the cell compositions described herein and/or reagents to produce and/or use the cell compositions may be comprised in a kit. In a non-limiting example, cells or reagents to manipulate cells may be comprised in a kit. In certain embodiments, cells that have reduced expression of B2M and/or CIITA, or a population of cells that comprises NKT cells that have reduced expression of B2M and/or CIITA, may be comprised in a kit. Such a kit may or may not have one or more reagents for manipulation of cells. Such reagents include small molecules, proteins, nucleic acids, antibodies, buffers, primers, nucleotides, salts, and/or a combination thereof, for example. Nucleic acids (DNA or RNA) or other agents that are capable of directly or indirectly reducing expression of B2M and/or CIITA may be included in the kit, such as shRNA or CRISPR guide RNA. Nucleic acids that encode one or more cytokines, or cytokines themselves, may be included in the kit. Proteins, such as cytokines or antibodies, including agonistic monoclonal antibodies, may be included in the kit. Substrates that comprise the antibodies, or naked substrates themselves, may be included in the kit. Cells that comprise antigen presenting cell activity or reagents to generate same may be included in the kit. Nucleotides that encode engineered receptors, such as chimeric antigen receptors or chimeric cytokine receptors or engineered T-cell receptors, may be included in the kit, including one or more reagents to generate same.

In particular aspects, the kit comprises the cell therapy of the disclosure and also another therapy for a particular medical condition, such as a cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for a subject and comprise respective second cancer therapies for the subject.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the composition and any other reagent containers in close confinement for commercial sale. Such containers may include injection or blow-molded plastic containers into which the desired vials are retained.

### EXAMPLES

### Example 1: Materials and Methods

### NKT-Cell Isolation, Expansion and In Vivo Injection.

Isolate PBMCs via apheresis. Buffy Coats (Gulf Coast Regional Blood Center) are obtained. Samples are diluted with equal volume of PBS. 15 ml Ficoll-Paque is placed in 50 ml centrifugation tube, and is carefully overlayed with 35 ml of the peripheral blood/PBS onto Ficoll-Paque without disturbance of the interface. The tubes are centrifuged at 800×g for 30 min at RT with no brake. The upper PBS layer is carefully aspirated, leaving about 10 mls of PBS. The PBMCs are carefully harvested at the PBS/Ficoll-Paque using a serological pipette. The harvested PBMCs are washed 3 times with 50 ml PBS by centrifugation at 800×g for 5 mins at RT. PBMCs are resuspended in 50 ml MACS buffer and count using trypan blue. Proceed to iNKT isolation.

Isolate NKT cells with Miltenyi microbeads. Cell number is first determined from the previous step. Cell suspension is centrifuged at 300×g for 10 minutes. The supernatant is aspirated completely. Cell pellet is resuspended in 400 μL of MACS buffer per 10 total cells. 100 μL of Anti-iNKT MicroBeads (Miltenyi Biotec) is added per 10 total cells. The cells and the MicroBeads are mixed well and incubated for 15 minutes in the refrigerator (2-8° C.). The cells are washed by adding 1-2 mL of MACS buffer per 10 cells and centrifuged at 300×g for 10 minutes. The supernatant is aspirated completely. Up to 10 cells are resuspended in 500 μL of MACS buffer. The column is placed in the magnetic field of a suitable MACS Separator. The column is prepared by rinsing with the appropriate amount of MACS buffer: LS: 3 mL. Cell suspension is applied onto the column. Flow-through containing unlabeled cells is collected. The column is washed with the appropriate amount of MACS buffer. Unlabeled cells that pass through: LS: 3×3 mL are collected. The column is then removed from the separator and placed on a suitable collection tube. The appropriate amount of MACS buffer is pipetted onto the column. The magnetically labeled cells are immediately flushed out by firmly pushing the plunger into the column. LS: 5 mL.

NKT primary stimulation including transduction. NKT cells are centrifuged at 400 g for 5 mins at RT and resuspended in 1 ml complete RPMI media and plated in 1 well of 24-well plate. Cells are counted and small aliquot is taken for purity staining at this step. PBMCs are counted. An appropriate amount of PBMCs is irradiated with 2.5 Gy by setting irradiator to Level 5, and irradiated for 10 minutes, 40 seconds. After irradiation, PBMCs are washed and resuspended at 5×10⁶ cells/mL. 1 ml of PBMCs (5 million cells) are added to NKT cells in 24-well plate. 100 ng/ml (2 μL) αGalCer (stock: 100 μg/mL), 200 IU/mL (2 μL) IL-2 (Stock: 200 IU/μL), and 10 ng/mL IL-21 are added. Cells are incubated at 37° C., 5% CO₂ for 10 days, and are fed with 200 IU/ml IL-2 and 10 ng/mL IL-21 every other day. Media is changed and/or wells are split as necessary. On day 8 of primary expansion, NKT cell transduction is performed as follows. After the transduction, cells are transferred to a 6-well G-Rex plate once NKT number exceeds 10×10⁶ cells and continue to expand for 10-12 days total. At the end of primary expansion, NKT cells can either be frozen or proceed to secondary stimulation.

NKT cell transduction. Retronectin-coated plate is prepared: i). Determine the number of wells needed for transduction; ii). Make a suspension of Retronectin at 7 ug/ml in PBS for each well and add 1 ml of Retronectin suspension to each well of a non-tissue culture coated plate; iii) Seal the edges of the plate with Parafilm and incubate overnight at 4° C. Alternatively, for same-day use, incubate Retronectin-coated plate for 4 hours at 37° C. The Retronectin-coated plate is then removed from 4° C. and warmed in hood for about 10 min. At the same time, retroviral supernatant(s) are thawed. Retronectin suspension is aspirated and discarded. 1 ml of retroviral supernatant is added to each well. The plate is centrifuged at 4600G for 1 hr, 30° C. NKT cells are collected and prepared at a concentration of 0.25×10⁶ cells/ml. IL-2 200 IU/ml and IL-21 10 ng/ml are added to NKT suspension. Retroviral supernatant is aspirated. NKT suspension is plated into each well for a final concentration of 0.5×10⁶ NKTs per well. The plate is spun at 400 g for 10 minutes. The plate is then incubated at 37° C., 5% CO₂ for 48 hours. On day 9 of primary expansion, transfer NKT cells into a 24-well tissue culture plate with fresh media. Wells are generally pooled together in order to maintain approximately 1×10⁶ cells/ml concentration.

NKT secondary expansion. Following end of primary stimulation/transduction, or working with primary-expanded frozen NKT cells, NKT cells are resuspended at 2×10⁶ cells/ml. If using PBMCs for secondary stimulation, frozen aliquot is thawed and irradiated at Level 5 for 10 minutes and 40 seconds. If using artificial APC (B-8-2), cells are resuspended at 1×10⁶ cells/ml and irradiated at Level 5 for 27 minutes. Irradiated cells are washed and co-cultured with NKT cells at a 1:5 NKT:PBMC or a 2:1 NKT:aAPC ratio in a 24 well plate. 100 ng/ml (2 μL) αGalCer (stock: 100 μg/mL), 200 IU/mL (2 μL) IL-2 (Stock: 200 IU/μL), and 10 ng/mL IL-21 are added. Cells are incubated at 37° C., 5% CO₂ for 10 days, and are fed with 200 IU/ml IL-2 and 10 ng/mL IL-21 every other day. Media is changed and/or wells are split as necessary. Cells are transferred to G-Rex 10 once NKT number exceeds 10×10⁶ cells and continue to expand for 10-12 days total.

Evaluate CAR.CD19 transduction efficiency and NKT cell purity. Single-color compensation controls are set up using 0.5-1×10⁶ NKT cells per FACS tube for each individual antibody, and are stained in a final volume of 50 ul. Cells are incubated for 20 minutes at 4° C., and are washed once with 2 ml 1×PBS, spun at 400×g for 5 minutes, and resuspended in 300 ul 1×PBS. For unstained control, 0.5-1×10⁶ NKT cells are set aside in an additional FACS tube. For experimental samples, 0.5-1×10⁶ NKT cells are transferred from culture into a FACS tube. Non-transduced cells are used as negative control. Cells are washed with 2 ml 1×PBS. 5 ul Alexa 647 anti-CAR.CD19 antibody is added and the cells are incubated at 4° C. off-light for 20 minutes. Cells are then washed thoroughly.

Day 0: Establish lymphoma xenografts using firefly luciferase/GFP+ CD19+ Daudi cells. NOD/SCID/IL2γnull (NSG) mice are maintained at the Small Animal Core Facility of Texas Children's Hospital and are treated according to the protocols approved by Baylor College of Medicine's Institutional Biosafety Committee and Institutional Animal Care and Use Committee (IACUC)—refer to animal research protocol number AN-5194. On Day 0, NSG mice are injected via tail vein with 2×10⁵ firefly luciferase/GFP+ Daudi cells to establish disease. Cells are washed with PBS. 300 ul PBS is added and the samples are run on LSRII or iQue. First, gate on live lymphocytes in the FSC vs SSC plot. Gate directly on CAR/CD19+ positive cells, using non-transduced NKT cells to set up the CAR+ gate.

Day 3: Inject CAR.CD19 transduced NKTs. Three days after injecting Daudi xenografts, NSG mice carrying Daudi tumors are injected via tail vein with 5×10⁶ CAR.CD19 NKT cells followed by intraperitoneal injection of IL-2 (2000 U/mouse) every other day for two weeks. Tumor size/distribution is monitored every week using bioluminescence imaging as follows. Just prior to imaging, each mouse is injected with 100 μL luciferin at 30 mg/mL via intraperitoneal injection. After 5 min, the mice are imaged using an IVIS® Lumina II Quantitative Fluorescent and Bioluminescent imaging system under a bioluminescent channel at Texas Children's Hospital, Small Animal Imaging Facility. Bioluminescence counts are then analyzed using Living Image® software.

### In Vitro Cytotoxicity Assay

Cultures of luciferase positive Daudi or Raji cells are established in RPMI-1640/GlutaMAX/10% (v/v) FBS. Luciferase expression is confirmed prior to beginning experiment and the number of target cells is determined to use in cytotoxicity assay (A standard curve is set up with 200,000 cells at the highest concentration, then 1:2 serial dilutions are performed and evaluated for luciferase expression. Ensure that the number of target cells used in assay falls within linear range of standard curve). A suspension of Daudi cells is prepared at 0.2×10⁶ cells/mL (or number of cells calculated based on standard curve) in RPMI/20% (v/v) FBS medium. 100 μL (20,000 cells) is plated in appropriate wells of black clear bottom 96-well plates. At least three wells are set up with target cells only and three wells are set up for media only controls. The wells are placed in 37° C. in a 5% CO₂-in-air, fully humidified atmosphere while effector cells are processed. Effector cells are harvested and counted. The cells are diluted to appropriate concentration for 10:1, 5:1, 2.5:1, and 1.25:1 effector:target ratios, ensuring that transduction rate is normalized across all CAR-transduced NKT cells. Effector cells are added to target for each concentration in triplicate. Cells are cultured for 6 hours at 37° C. in a 5% CO₂-in-air, fully humidified atmosphere. Tecan Spark 10M plate reader is set up to warm to 37° C., bioluminescence signal is read, and an acquisition template is set up. 100 μL of medium is carefully removed from all wells of each plate while avoiding contact with base of wells. Immediately prior to use, required amount of 1.5 mg/ml working stock of luciferin is prepared. 100 ul of luciferin is added to all wells of each plate. The plates are incubated for 5 minutes at 37° C. in a 5% CO₂-in-air, fully humidified atmosphere. Plates are removed from incubator, the lid are then removed, and bioluminescence is read using Tecan Spark 10M plate reader. For data analysis: acquire data and calculate percentage killing/lysis as:

$$\frac{( Total luciferase ) - ( x ) \times 100 \%}{( Total luciferase ) - ( Spontaneous luciferase )}$$

### Retroviral Constructs and Retrovirus Production.

CAR.CD19, CAR.GD2, and CAR.GPC3 constructs are made as previously described (Heczey et al., 2014; Pule et al., 2005) and contained a scFv from the CD19-specific antibody FMC-63 or the GD2-specific antibody 14G2a connected via a short spacer derived from the IgG1 hinge region to the transmembrane domain derived from CD8a, followed by signaling endodomain sequences of 4-1BB fused with z chain.

Cloning and Sequence Information for CAR19.IL2SP-Opti15.amiR Construct

The primer sequences are from Sigma-Aldrich and are designed using “Primer BLAST” tool from the NCBI. Template is the CAR19.15 vector. Table 2 below shows the cloning primers and DNA fragments synthesized. Table 3 is the sequencing primers.

TABLE 2
Cloning primers and DNA fragments synthesized

| | SEQ ID | |
|---|---|---|
| Name | NO: | Sequence |
| F-car19 | 27 | TGCCATGGAGTTTGGGCTGAGCTGGC |
| R-zeta | 28 | GCGAGGGGGCAGGGCCTGCAT |
| Opti-15 | 29 | CAGTGTACTAATTATGCTCTCTTGAAATTGGCTGGAGATGTTGAGAGCAATCCCGGGCCC |
| (synthesized | | ATGAGAATCAGCAAGCCCCACCTGAGATCCATCAGCATCCAGTGCTACCTGTGCCTGCTG |
| by IDT) | | CTGAACAGCCACTTTCTGACAGAGGCCGGCATCCACGTGTTCATCCTGGGCTGTTTTTCT |
| | | GCCGGCCTGCCTAAGACCGAGGCCAACTGGGTTAACGTGATCAGCGACCTGAAGAAGATC |
| | | GAGGACCTGATCCAGAGCATGCACATCGACGCCACACTGTACACCGAGAGCGACGTGCAC |
| | | CCTAGCTGTAAAGTGACCGCCATGAAGTGCTTTCTGCTGGAACTGCAAGTGATCAGCCTG |
| | | GAAAGCGGCGACGCCAGCATCCACGACACCGTGGAAAACCTGATCATCCTGGCCAACAAC |
| | | AGCCTGAGCAGCAACGGCAATGTGACCGAGTCCGGCTGCAAAGAGTGCGAGGAACTGGAA |
| | | GAGAAGAATATCAAAGAGTTCCTGCAGAGCTTCGTGCACATCGTGCAGATGTTCATCAAC |
| | | ACCAGCTGAGAGCGCTTG |
| miR30- | 30 | AGAGCGCTTGTTTGAATGAGGCTTCAGTACTTTACAGAATCGTTGCCTGCACATCTTGGA |
| B2M | | AACACTTGCTGGGATTACTTCGACTTCTTAACCCAACAGAAGGCTCGAGAAGGTATATTG |
| fragment | | CTGTTGACAGTGAGCGAAGGTTTGAAGATGCCGCATTTTAGTGAAGCCACAGATGTAAAA |
| (synthesized | | TGCGGCATCTTCAAACCTCTGCCTACTGCCTCGGACTTCAAGGGGCTAGAATTCGAGCAA |
| by IDT, | | TTATCTTGTTTACTAAAACTGAATACCTTGCTATCTCTTTGATACATTTTTACAAAGCTG |
| underlined | | AATTAAAATGGTATAAATTAAATCACTTTGTTAACATGATGTCGACCT |
| text = | | |
| shRNA | | |
| sequence) | | |
| miR155- | 31 | ATGTCGACCTGGAGGCTTGCTGAAGGCTGTATGCTGTTTGTAGGCACCCAGGTCAGTGTT |
| CIITA | | TTGGCCACTGACTGACACTGACCTGTGCCTACAAACAGGACACAAGGCCTGTTACTAGCA |
| fragment | | CTCACATGGAACAAATGGCCGTCGACACCTCGAGAT |
| (synthesized | | |
| by IDT; | | |
| underlined | | |
| text = | | |
| shRNA | | |
| sequence) | | |

TABLE 3
Sequencing primers:

| | SEQ ID | |
|---|---|---|
| Name | NO: | Sequence |
| F-CAR19 | 32 | CACCGCCCTCAAAGTAGAC |
| F-Z | 33 | ATGGCCTTTACCAGGGTCTCAG |
| F-OPTI-15 | 34 | CGAGGAACTGGAAGAGAAGAAT |
| R-VECTOR | 35 | TCGTACTCTATAGGCTTCAGC |

### Proliferation and Apoptosis Assays

NKTs are labeled with CellTrace Violet (CTV; Thermo Fisher, Waltham, Mass.) and stimulated with αGalCer-pulsed B-8-2 cells. Cell proliferation is examined on day 6 by measuring CTV dilution using flow cytometry. Early and late apoptosis is measured on day 3 post-NKT stimulation by staining for annexin-V and 7-AAD (BD Biosciences, Franklin Lakes, N.J.), respectively, followed by flow cytometry.

Multiplex cytokine quantification assay CD19-CAR-NKTs are stimulated for 24 hours by Daudi lymphoma cells at a 1:1 ratio. Supernatants are collected and analyzed using the MILLIPLEX MAP Human Cytokine/Chemokine Immunoassay panel (Millipore) for Luminex® analysis according to the manufacturer's protocol.

### Flow Cytometry.

Immunophenotyping is performed using the following mAbs to: HLA-C EMR8-5, CD 1d CD1d42, CD86 2331, 4-1BBL C65-485, OX40L ik-1, CD3 OKT, Va24-Ja18 6B11, CD4 SK3, CD62L DREG-56, CD134 ACT35, CD137 4B4-1, PD-1 EH12.1, GATA3 L50-823 (BD Biosciences), LAG-3 Polyclonal, TEVI-3 344823 (R&D System), and rabbit anti-LEF1 EP2030Y mAb (ABCAM). BD or R&D-suggested fluorochrome and isotype-matching Abs is used as negative controls. The expression of CAR.CD19 on NKTs is determined using anti-Id (clone 136.20.1) CD19-CAR specific mAb (Torikai H, et al. Toward eliminating HLA class I expression to generate universal cells from allogeneic donors. fi/oo<i.2013; 122(8):1341-1349) and goat anti-mouse IgG (BD Biosciences).

### NKT-Cell Phenotypic Analysis

NKT-cell phenotype is assessed using monoclonal antibodies (mAbs) for CD3 (UCHT1), Vα24-Jα18 (6B11), CD4 (RPA-T4), granzyme B (GB11), CD62L (DREG-56; BD Biosciences, San Jose, Calif.), Vβ11 (C21; Beckman Coulter, Brea, Calif.), and IL-21R (17A12; BioLegend, San Diego, Calif. and BD Biosciences). CD19-CAR expression by transduced NKTs is detected using anti-Id mAb (clone 136.20.1) (25), a gift from Dr. B. Jena (MD Anderson Cancer Center, Houston, Tex.). Intracellular staining is performed using a fixation/permeabilization solution kit (BD Biosciences) with mAbs for Bcl2 (N46-467; BD Biosciences) and BIM (Y36; Abcam, Cambridge, Mass.) followed by staining with a secondary goat anti-rabbit IgG-AF488 mAb (Abcam). Phosflow staining is performed using Cytofix buffer (BD Biosciences) and Perm buffer III (BD Biosciences) with mAb for Stat3 (pY705; Clone 4; BD Biosciences). Detection of Stat3 phosphorylation is performed after 15 minutes of treatment with IL-21. Fluorochrome- and isotype-matching antibodies suggested by BD Biosciences or R&D Systems is used as negative controls.

Analysis is performed on an LSR-II 5-laser flow cytometer (BD Biosciences) using BD FACSDiva software version 6.0 and FlowJo 10.1 (Tree Star, Ashland, Oreg.).

### Gene Expression Analysis

Total RNA is collected using the Direct-zol™ RNA MiniPrep Kit (Zymo Research, Irvine, Calif.). Gene expression analysis is performed using the Immunology Panel version 2 (NanoString, Seattle, Wash.) with the nCounter Analysis System by the BCM Genomic and RNA Profiling Core. Data is analyzed using nSolver 3.0 software (NanoString). Differences in gene expression levels between CD62L+ and CD62L− subsets in the two culture conditions are evaluated using the paired moderated t-statistic of the Linear Models for Microarray Data (Limma) analysis package (26).

### In Vivo Experiments

NSG mice are obtained from the Jackson Laboratory and maintained at the BCM animal care facility. Mice are injected intravenously (IV) with 2×10⁵ luciferase-transduced Daudi lymphoma cells to initiate tumor growth. On day 3, mice are injected IV with 4×10×10⁶ CD19-CAR-NKTs followed by intraperitoneal (IP) injection of IL-2 (1,000 U/mouse) only or a combination of IL-2 (1,000 U/mouse) and IL-21 (50 ng/mouse) every other day for two weeks. Tumor growth is assessed once per week by bioluminescent imaging (Small Animal Imaging core facility, Texas Children's Hospital).

### Statistics

The Shapiro-Wilk test is used to assess normality of continuous variables. Normality is rejected when the P value is less than 0.05. For non-normally distributed data, the Mann-Whitney U test is used to evaluate differences in continuous variables between two groups. To evaluate differences in continuous variables, a two-sided paired Student's t-test is used to compare two groups, one-way ANOVA with post-test Bonferroni correction is used to compare more than two groups, and two-way ANOVA with Sidak's post-hoc test is used to compare in a two-by-two setting. Survival is analyzed using the Kaplan-Meier method with the log-rank (Mantel-Cox) test to compare two groups. Statistics are computed using GraphPad Prism 7 (GraphPad Software, San Diego, Calif.). Differences are considered significant when the P value was less than 0.05.

### Example 2: amiR Versus Pol III Promoter-Driven shRNA for HLA Class I/II Knockdown and Co-Expression with CAR19 in NKTS

To limit rejection of NKT cells by the immune system of an allogeneic host, recombinant constructs that incorporate U6 promoter-driven shRNA sequences against β2-microglobulin (B2M) and the invariant chain (Ii) (a.k.a. CD74) or the class II transactivator (CIITA) are designed to achieve knock-down of HLA class I and class II, respectively, in NKT cells. Constructs comprising the 7SK and the H1 polymerase III promoters instead of the U6 promoter are also designed and evaluated.

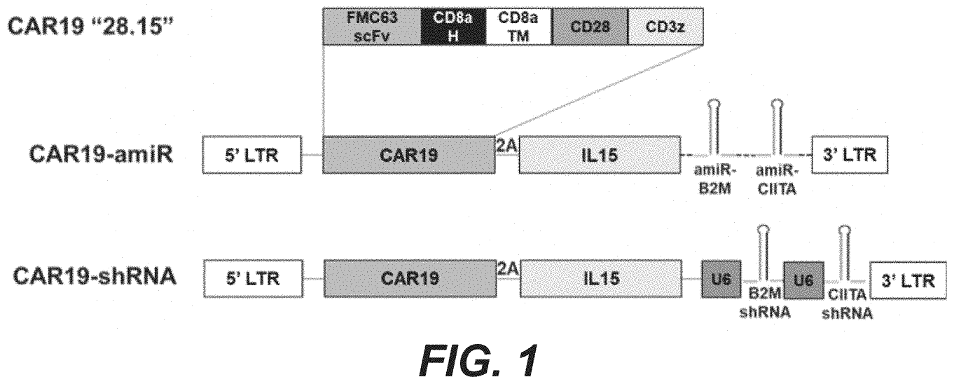

Meanwhile, experiments are carried out to evaluate the feasibility of using amiR scaffolds (e.g., amiR155 and amiR30) to support expression of B2M-shRNA sequences from within CAR19. The CAR19 construct is shown in FIG. 1. The goal is to evaluate how this approach compares to use of polymerase III promoter-driven shRNA in terms of impact on CAR expression and ability to effectively suppress expression of HLA class I and/or II in transduced NKTs.

In FIG. 2, NKT cells are transduced with CAR19 constructs containing scrambled (scr.) or B2M-specific shRNA driven by the U6, H1, or 7SK promoter or embedded in the miR155 scaffold. CAR expression was evaluated 2 days post-transduction. FIG. 2 shows that in NKT cells from a representative donor, incorporation of either promoter- or miR-driven shRNA at the 3′ end of the CAR19 construct similarly reduced the level of CAR expression regardless of shRNA specificity.

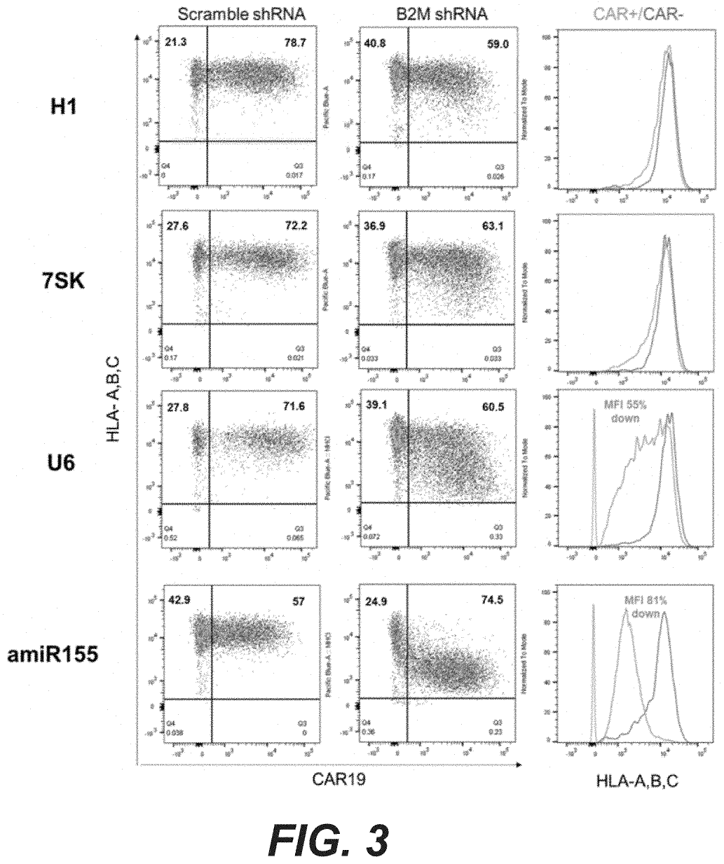

In FIG. 3, NKT cells are transduced with CAR19 constructs containing scrambled (scr.) or B2M-specific shRNA driven by the H1, 7SK, or U6 promoter or embedded in amiR155 as indicated. CAR and HLA-A,B,C expression are evaluated 2 days post-transduction. FIG. 3 shows that B2M shRNA expression supported by amiR155 from within CAR19 yields the greatest level of HLA-A,B,C knockdown compared to the three polymerase III-driven promoters evaluated.

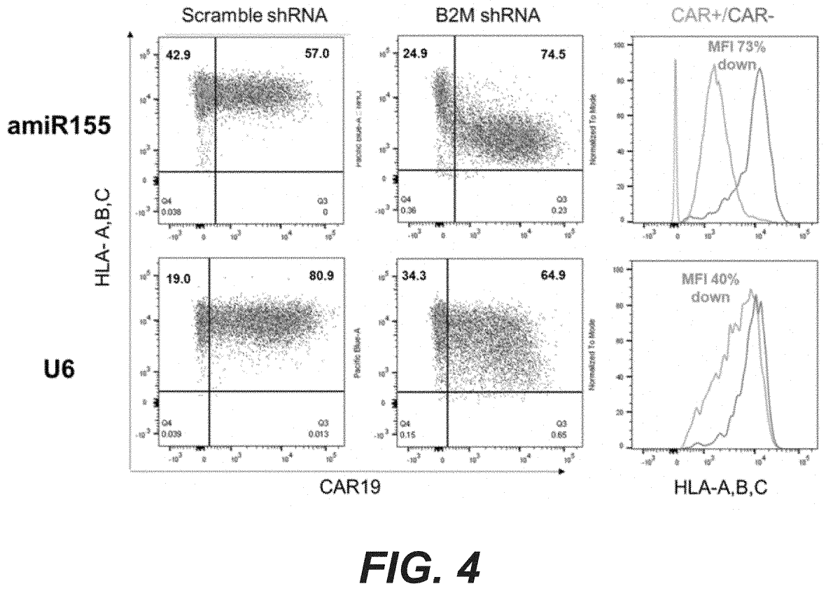

In FIG. 4, NKT cells are transduced with CAR19 constructs containing scrambled (scr.) or B2M-specific shRNA driven by the U6 promoter or embedded in amiR155 as indicated. CAR and HLA-A,B,C expression are evaluated 14 days post-transduction. FIG. 4 shows that the amiR155-B2M shRNA construct mediates effective long term (14 days post-transduction) suppression of HLA-A,B,C expression, demonstrating a greater degree of knockdown than the U6-B2M shRNA construct.

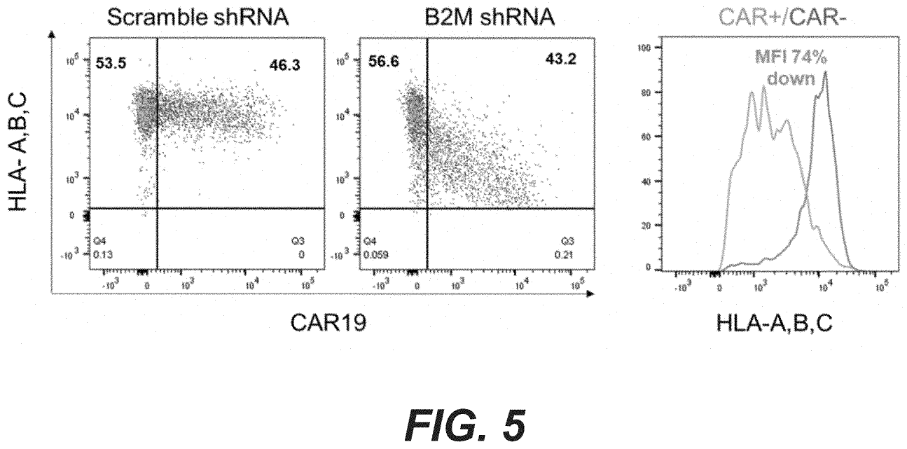

In FIG. 5, NKT cells are transduced with CAR19 constructs containing scrambled (scr.) or B2M-specific shRNA embedded in amiR30 as indicated. CAR and HLA-A,B,C expression are evaluated seven days post-transduction. FIG. 5 shows that the amiR30-B2M shRNA construct mediates effective suppression of HLA-A,B,C expression as assessed seven days post-transduction, demonstrating a comparable degree of knockdown to the amiR155-B2M shRNA construct.

Taken together, these experiments demonstrate that incorporation of either promoter- or miR-driven shRNA at the 3′ end of the CAR19 construct similarly reduces the level of CAR expression regardless of shRNA specificity. B2M shRNA expression supported by amiR155 from within CAR19 yields the greatest level of HLA-A,B,C knockdown compared to the U6, H1, and 7SK polymerase III-driven promoters. The amiR155-B2M shRNA construct mediates more effective and stable suppression of HLA-A,B,C expression compared to the U6-B2M shRNA construct. The amiR30-B2M shRNA construct mediates effective suppression of HLA-A,B,C expression as assessed seven days post-transduction, demonstrating a comparable degree of knockdown to the amiR155-B2M shRNA construct.

### Example 3: Screening amiR-shRNA Target Sequences for B2M, CIITA, and CD74

In this example, different shRNA candidate sequences targeting B2M, CIITA, and CD74 are screened as detailed below. The shRNA sequences are either selected from a set of validated shRNAs available through Sigma (1 in lists below) or designed using the Invitrogen RNAi tool (2 in lists below). This screening approach allows for selection of the shRNA sequence in each case that, in conjunction with amiR155 within CAR19, mediated the most efficient knockdown of HLA-A,B,C (for B2M shRNA) and HLA-DR,DP,DQ (for CIITA and CD74 shRNA) in transduced NKT cells.

Table 4 provides the sequences for the shRNA candidates:

TABLE 4
Sequences for the shRNA candidates

| | | SEQ | |
|---|---|---|---|
| Target | shRNA | ID NO | Sequence |
| HLA | B2M #1 | 1 | ctggtctttctatctcttgta¹ |
| class 1 | B2M #2 | 2 | cagcagagaatggaaagtcaa¹ |
| | B2M #3 | 3 | ccgtgtgaaccatgtgacttt¹ |
| | B2M #4 | 4 | agttaagcgtgcataagttaa¹ |
| | B2M #5 | 5 | tagagtttggctcacagtgta¹ |
| | B2M #6 | 6 | aggtttgaagatgccgcattt¹ |
| HLA | CIITA #1 | 7 | ttgtacaagcttagcctgagc¹ |
| class II | CIITA #2 | 8 | tagggtactttgatgtctgcg¹ |
| | CIITA #3 | 9 | gttaagaagctccaggtagcc¹ |
| | CIITA #4 | 10 | ttccatgtcacacaacagect² |
| | CIITA #5 | 11 | tttggaagcttgttggagacc² |
| | CIITA #6 | 12 | tttgtaggcacccaggtcagt² |
| | CIITA #7 | 13 | atctcaggctgatccgtgaat² |
| | CIITA #8 | 14 | tggagaagtactttctctgtg² |
| | CIITA #9 | 15 | ttagctgtttccctgctaagg² |
| | CIITA #10 | 16 | tgaactcaaaccctggacctg² |
| HLA | CD74 #1 | 17 | ccaccaagtatggcaacatga¹ |
| class II | CD74 #2 | 18 | ccacacagctacagctttctt¹ |
| | CD74 #3 | 19 | caagtcggaacagcagataac¹ |
| | CD74 #4 | 20 | cgcgaccttatctccaacaat¹ |
| | CD74 #5 | 21 | gaccatagactggaaggtctt¹ |
| | CD74 #6 | 22 | cctttgtagctttcacttcca² |
| | CD74 #7 | 23 | gaacctgagacaccttaagaa² |
| | CD74 #8 | 24 | gcaccattggctcctgtttga² |
| | CD74 #9 | 25 | tcacagcagcctccaacacaa² |
| | CD74 #10 | 26 | caacacaaggctccaagacct² |

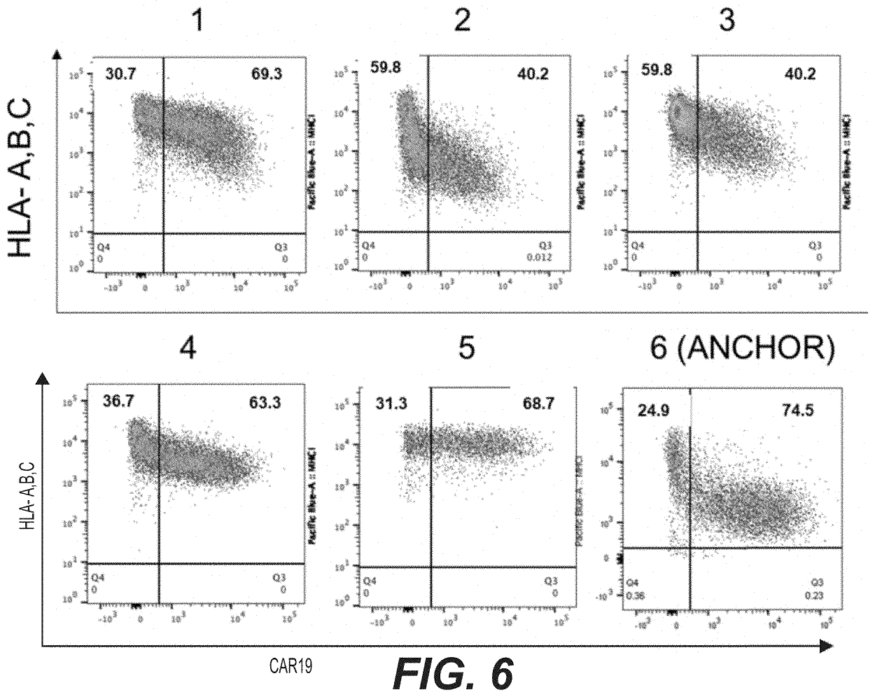

In FIG. 6, NKT cells are transduced with CAR19 constructs containing B2M-specific shRNA (5 distinct candidate sequences and previously evaluated shRNA sequence used in ANCHOR product) embedded in amiR155. CAR and HLA-A,B,C expression are evaluated 12 days post-transduction. The results show variation in HLA-A,B,C knockdown level depending on the specific shRNA sequence used to target B2M.

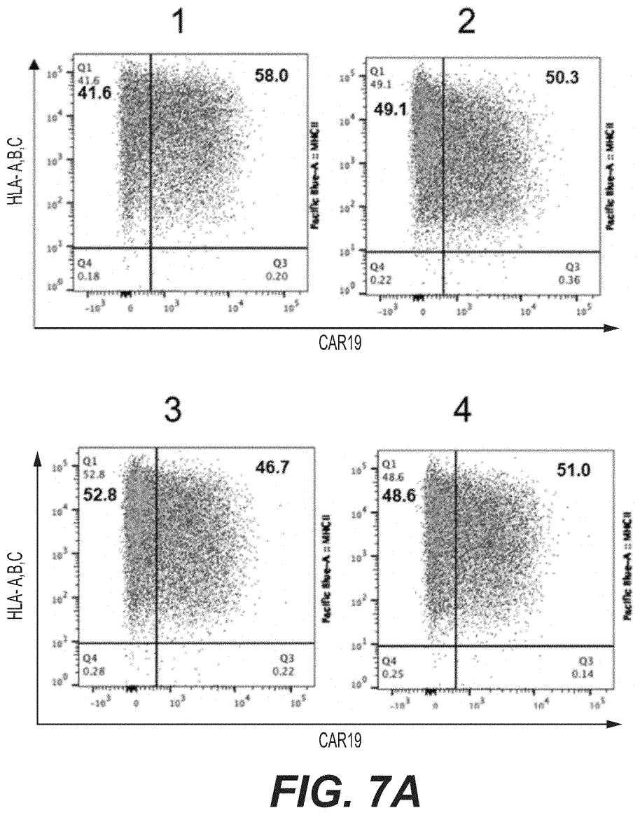

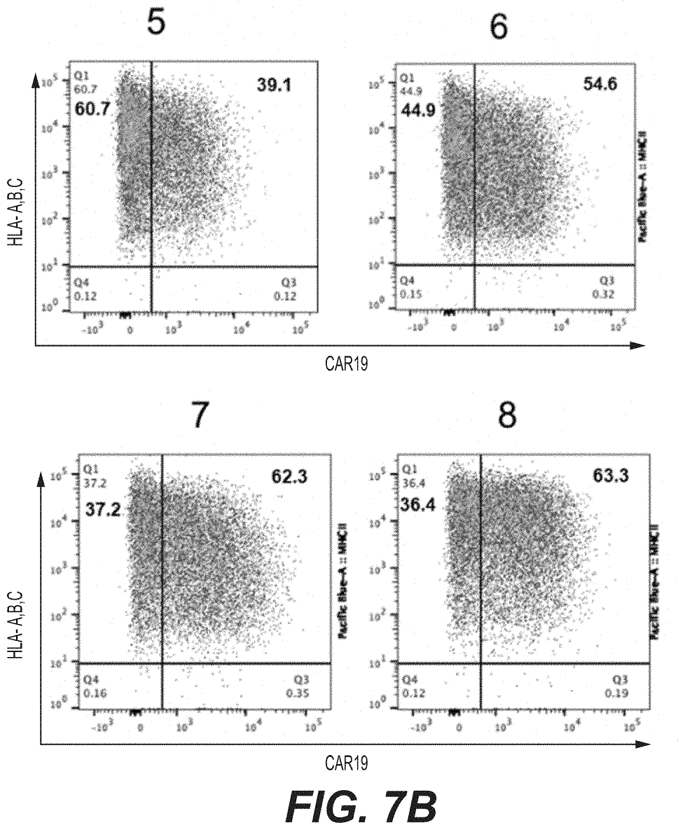

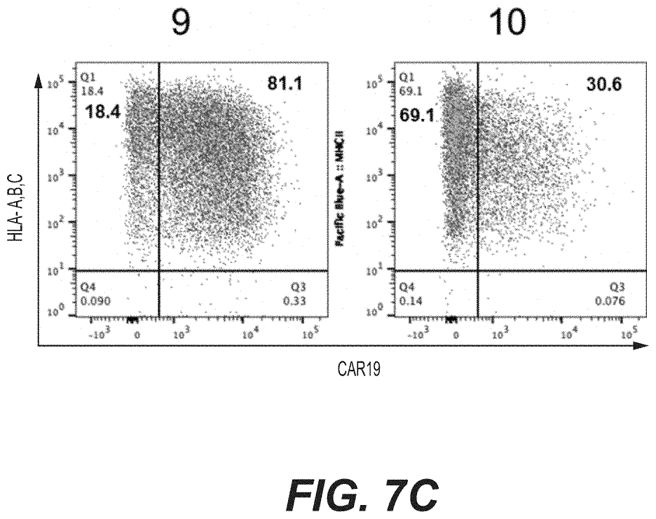

In FIG. 7, NKT cells are transduced with CAR19 constructs containing CIITA-specific shRNA (10 distinct candidate sequences) embedded in amiR155. CAR and HLA-DR,DP,DQ expression are evaluated 12 days post-transduction. The results show variation in HLA-DR,DP,DQ knockdown level depending on the specific shRNA sequence used to target CIITA.

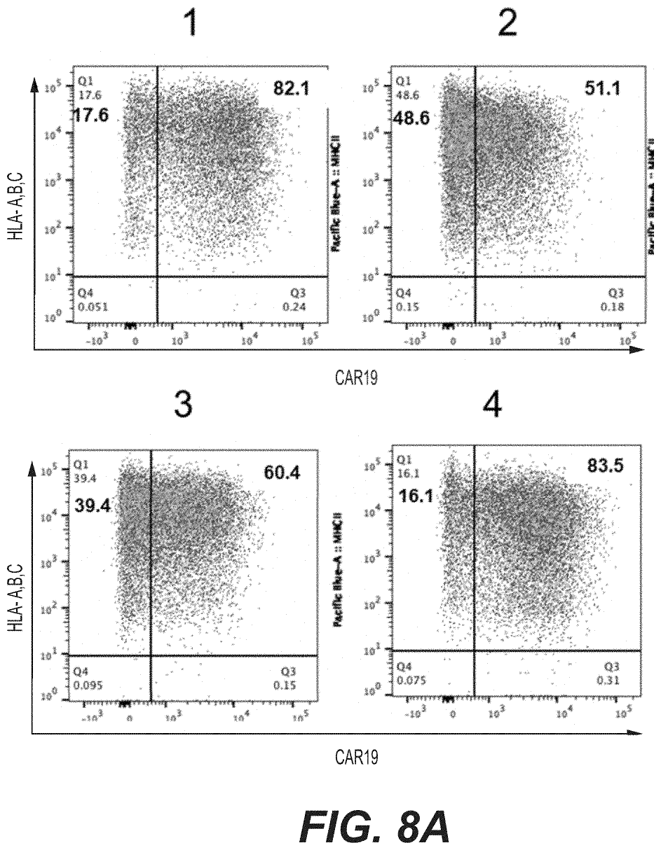

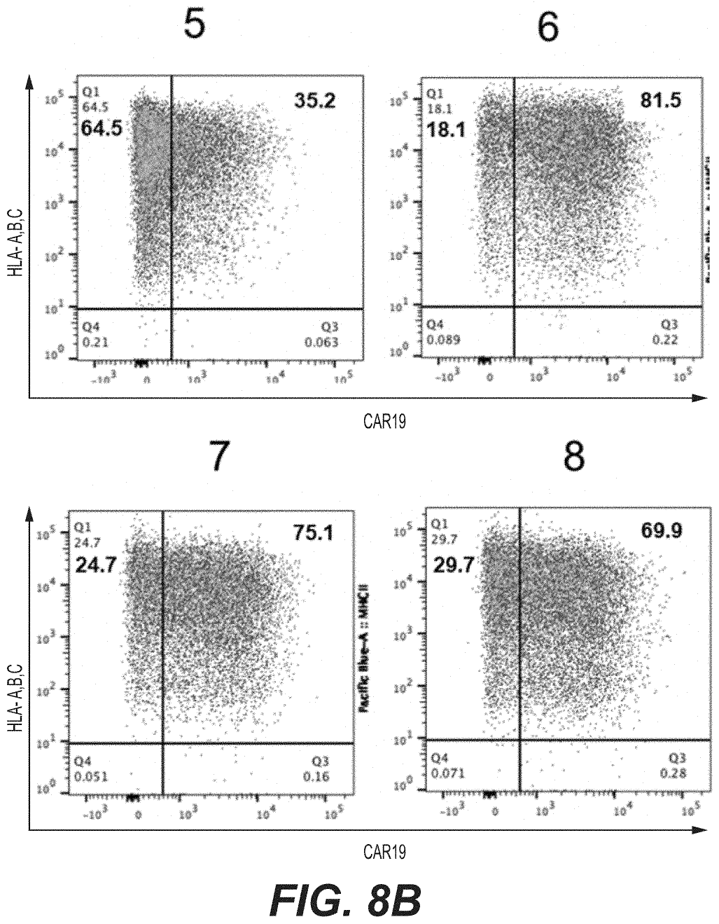

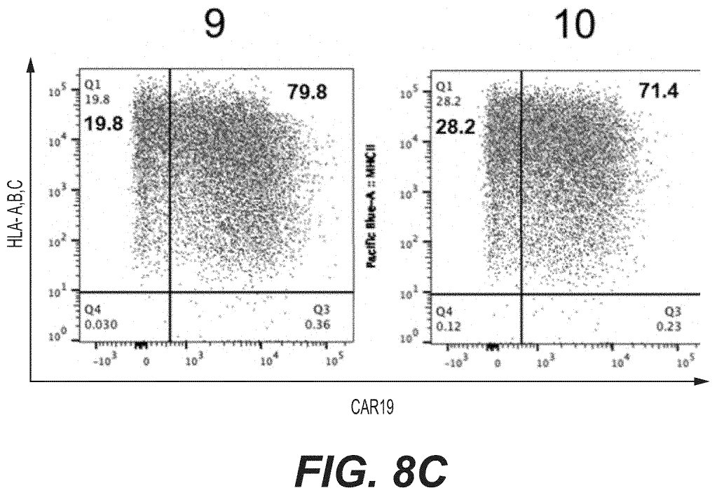

In FIG. 8, NKT cells are transduced with CAR19 constructs containing CD74-specific shRNA (10 distinct candidate sequences) embedded in amiR155. CAR and HLA-DR,DP,DQ expression are evaluated 12 days post-transduction. The results show variation in HLA-DR,DP,DQ knockdown level depending on the specific shRNA sequence used to target CD74.

Table 5 summarizes the quantification of HLA class I or II knockdown efficiency for the shRNA candidates evaluated in FIGS. 6-8.

TABLE 5

| | | | Q1: | Q2: | |
|---|---|---|---|---|---|
| | | | CAR19−, | CAR19+, | |
| | | SEQ | HLA I or | HLA I or | HLA I or II |
| | | ID | II + Mean | II + Mean | KD |
| Target | shRNA | NO | HLA I or II | HLA I or II | efficiency |
| HLA | CD74 #1 | 17 | 18293 | 15206 | 0.168753075 |
| class II | CD74 #2 | 18 | 16258 | 12099 | 0.255812523 |
| | CD74 #3 | 19 | 16811 | 15061 | 0.104098507 |
| | CD74 #4 | 20 | 15112 | 9676 | 0.359714134 |
| | CD74 #5 | 21 | 13606 | 12063 | 0.11340585 |
| | CD74 #6 | 22 | 20337 | 17670 | 0.131140286 |
| | CD74 #7 | 23 | 13519 | 10142 | 0.249796583 |
| | CD74 #8 | 24 | 13605 | 9599 | 0.29445057 |
| | CD74 #9 | 25 | 18403 | 11205 | 0.391131881 |
| | CD74 #10 | 26 | 17837 | 14136 | 0.207490049 |
| HLA | CIITA #1 | 7 | 16997 | 13119 | 0.22815791 |
| class II | CIITA #2 | 8 | 12275 | 5915 | 0.518126273 |
| | CIITA #3 | 9 | 14039 | 9201 | 0.34461144 |
| | CIITA #4 | 10 | 12166 | 7987 | 0.343498274 |
| | CIITA #5 | 11 | 13939 | 7670 | 0.449745319 |
| | CIITA #6 | 12 | 11472 | 5084 | 0.556834031 |
| | CIITA #7 | 13 | 12501 | 6707 | 0.463482921 |
| | CIITA #8 | 14 | 16581 | 13645 | 0.177070141 |
| | CIITA #9 | 15 | 15062 | 9191 | 0.389788873 |
| | CIITA #10 | 16 | 14499 | 7443 | 0.486654252 |
| HLA | B2M#1 | 1 | 9158 | 4449 | 0.514195239 |
| class I | B2M #2 | 2 | 4777 | 743 | 0.844463052 |
| | B2M #3 | 3 | 7853 | 3262 | 0.584617344 |
| | B2M #4 | 4 | 9236 | 3356 | 0.636639238 |
| | B2M #5 | 5 | 11634 | 10858 | 0.066701049 |
| | B2M #6 | 6 | 11281 | 2128 | 0.81 |

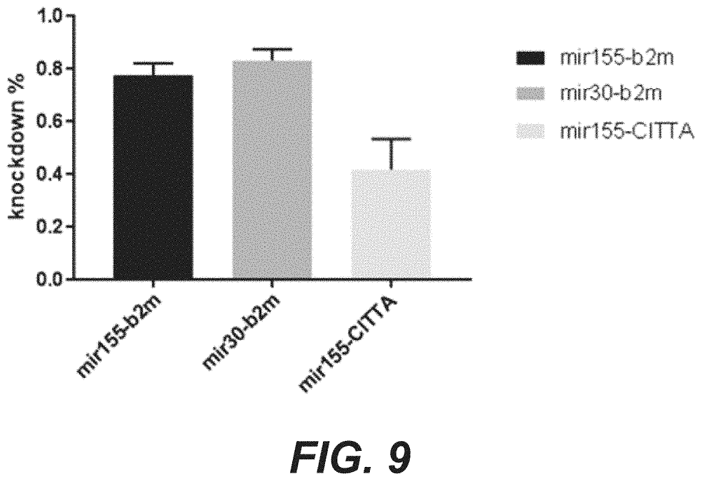

In FIG. 9, NKT cells are transduced with CAR19.15 constructs containing single amiR-embedded shRNA targeting B2M (using shRNA sequence from ANCHOR) or CIITA (using candidate sequence #6) as indicated. Knockdown efficiency is evaluated four days post-transduction. N=4 donors (BL #62, 80, 81, 83). Table 6 below presents the data corresponding to FIG. 9.

TABLE 6

| | KD % | KD % | KD % | KD % |
|---|---|---|---|---|
| | Donor 1 | Donor 2 | Donor 3 | Donor 4 |
| miR155-B2M | 0.81 | 0.745552 | 0.822627 | 0.736333 |
| miR30-B2M | 0.88 | 0.825983 | 0.848333 | 0.779514 |
| miR155-CIITA | 0.55 | 0.338715 | 0.48215 | 0.304001 |

Taken together, these experiments demonstrates the selection of the best shRNA candidates for B2M, CIITA, and CD74. For HLA class II knockdown, CIITA is selected over CD74 for shRNA targeting.

### Example 4: Improving IL15 Production by NKTS Expressing CAR19-amiR Constructs

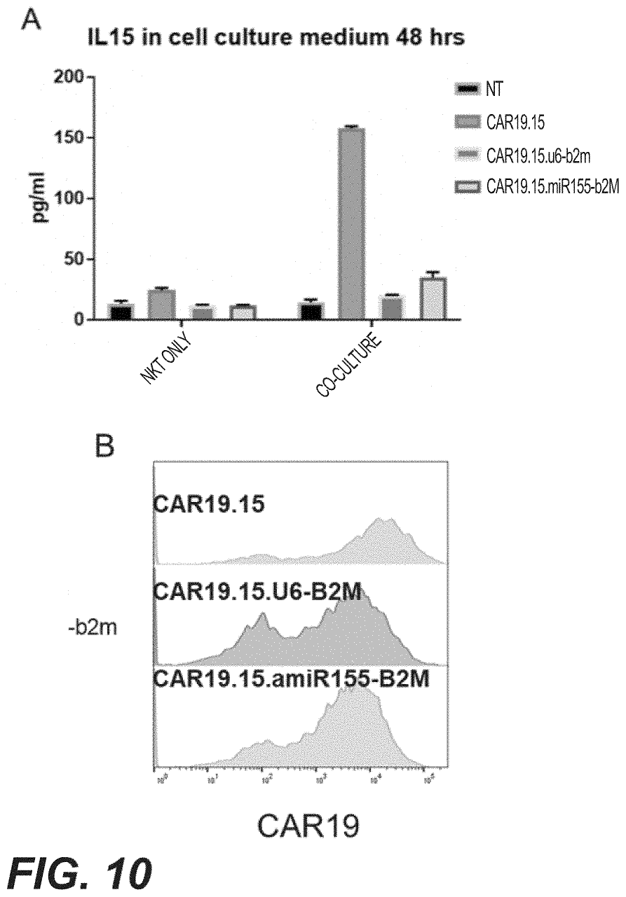

Efficient co-expression of IL15 from the CAR19 construct is important for promoting survival and anti-tumor activity of transduced NKTs. An IL15 ELISA is performed and the results indicate that NKTs expressing CAR19.15 with either U6-driven B2M shRNA or miR155-embedded B2M shRNA produce significantly reduced levels of IL15 compared to NKTs expressing the original CAR19.15 (FIG. 10 panel A). This reduction in IL15 levels also corresponds to a lower level of CAR expression from NKTs expressing these constructs (FIG. 10 panel B). Table 7 below presents the data corresponding to FIG. 10, panel A.

TABLE 7

| | NKT only | | | Lymphoma cell co-culture | | |
|---|---|---|---|---|---|---|
| NT | 15.46921 | 10.30059 | 14.22287 | 12.82991 | 13.37977 | 17.48534 |
| CAR19.15 | 26.8695 | 23.71701 | 23.60704 | 159.0543 | 156.4516 | 159.1642 |
| CAR19.15.U6-B2M | 11.91349 | 12.5 | 10.99707 | 19.83138 | 20.60117 | 20.71114 |
| CAR19.15.miR155-B2M | 12.31672 | 11.1437 | 11.84018 | 31.70821 | 34.42082 | 39.8827 |

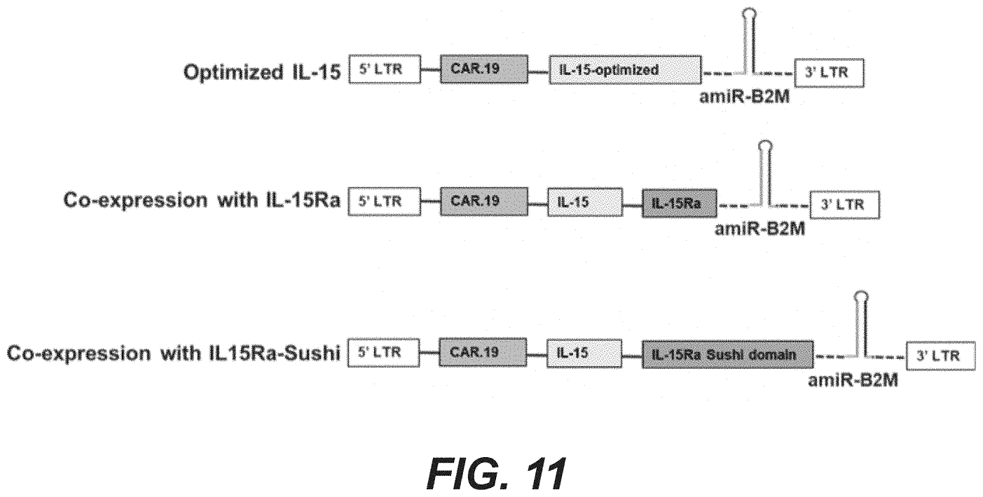

In order to address this issue, a set of three constructs (FIG. 11) are designed with modifications aimed to improve IL15 expression (codon-optimized IL15) or biological potency/activity (IL15 expressed in conjunction with IL15Ra or the IL15Ra Sushi domain). The NKT cells are transduced with the new constructs and evaluated for the impact on IL15 production.

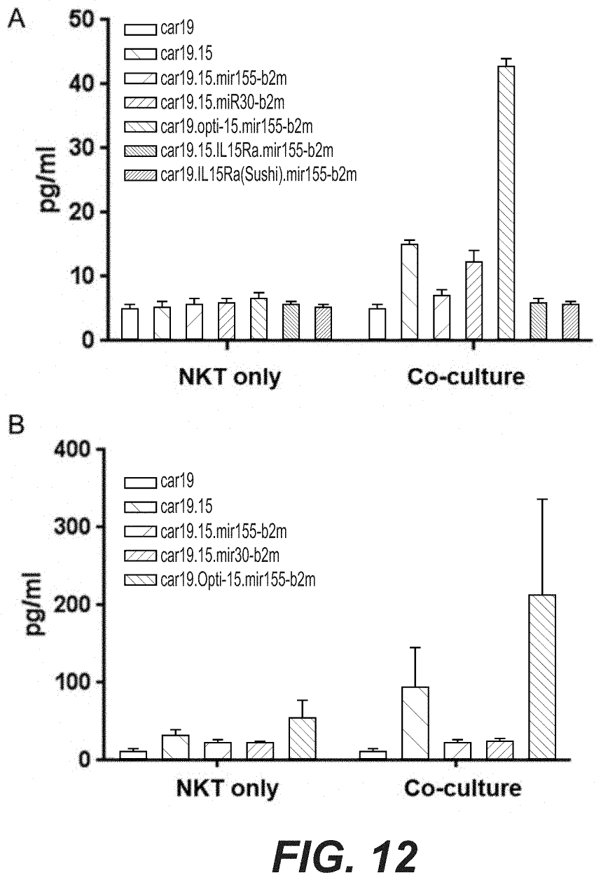

FIG. 12 shows that expression of codon-optimized (opti) IL15 from CAR19 construct with amiR155-driven B2M shRNA boosts secretion of IL15 following co-culture with CD19+ tumor cells. Table 8 below presents the data corresponding to FIG. 12, panel A. Table 9 below presents the data corresponding to FIG. 12, panel B.

TABLE 8

| | NKT only | | | Lymphoma cell co-culture | | |
|---|---|---|---|---|---|---|
| CAR19 | 5.45675 | 5.315279 | 5.153597 | 5.315279 | 5.537591 | 5.537591 |
| CAR19.15 | 5.638642 | 5.921584 | 4.769604 | 15.40016 | 14.67259 | 15.46079 |
| CAR19.15.miR155-B2M | 5.578011 | 6.669361 | 5.194018 | 7.134196 | 6.729992 | 7.881973 |
| CAR19.15.miR30-B2M | 5.881164 | 6.5481 | 6.042846 | 10.85287 | 12.20695 | 14.12692 |
| CAR19.opti15.miR155-B2M | 6.063056 | 6.143897 | 7.396928 | 41.81487 | 43.06791 | 43.75505 |
| CAR19.15.IL15Ra.miR155-B2M | 5.679062 | 5.820534 | 5.840744 | 6.50768 | 6.204527 | 5.679062 |
| CAR19.15.IL15Ra(Sushi).miR155- | 5.638642 | 5.679062 | 5.699272 | 5.941795 | 6.042846 | 5.901374 |
| B2M | | | | | | |

TABLE 9

| | NKT only | | | Lymphoma cell co-culture | | |
|---|---|---|---|---|---|---|
| CAR19 | 12.95922 | 12.19217 | 15.30077 | 14.61445 | 13.5648 | 11.62697 |
| CAR19.15 | 40.16956 | 26.96811 | 32.86233 | 150.5854 | 54.50141 | 84.77998 |
| CAR19.15.miR155-B2M | 27.33145 | 24.30359 | 23.25394 | 23.98062 | 24.78805 | 27.85628 |
| CAR19.15.miR30-B2M | 25.43399 | 24.86879 | 24.22285 | 25.03028 | 26.76625 | 29.06742 |
| CAR19.opti15.miR155-B2M | 66.69358 | 33.14493 | 69.03512 | 344.3682 | 97.69883 | 194.025 |

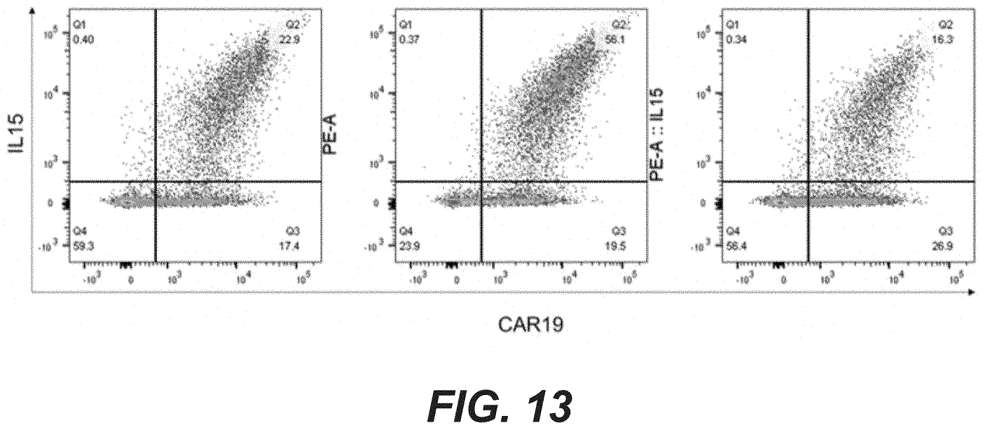

FIG. 13 shows that co-expression of IL15-IL15Ra from CAR19.15 promotes surface expression of IL15 by transduced NKTs via binding to IL15Ra. The data is from three donors.

Taken together, these experiments demonstrate that IL15 secretion and CAR expression are lower in NKTs expressing CAR19.15 construct with U6- or amiR155-driven B2M shRNA versus original CAR19.15. Expression of codon-optimized (opti) IL15 from CAR19 construct with amiR155-driven B2M shRNA boosts secretion of IL15 in NKTs. However, the effect was variable in three donors tested. Co-expression of IL15-IL15Ra from CAR19.15 promotes surface expression of IL15 by transduced NKTs via binding to IL15Ra. This binding may promote effective trans-presentation of IL15 to neighboring/target cells expressing the IL2R-beta and common gamma chains.

### Example 5: Evaluation of Double Knockdown Constructs: amiR-Embedded shRNA Sequences Co-Expressed with CAR19 and Optimized IL15

To minimize rejection of CAR19 NKTs in an allogeneic patient, a construct is designed to knock down HLA class I and II simultaneously using amiR-embedded shRNA sequences to target B2M (class I) and CIITA (class II). The best performing B2M and CIITA-specific shRNAs are selected and evaluated in the single knockdown screening for inclusion in the double knockdown construct: the B2M shRNA target sequence is the same as the one used in the ANCHOR product and is embedded within amiR30, and CIITA shRNA candidate #6 is embedded within amiR155. Codon-optimized IL15 is also integrated to maximize IL15 secretion by NKT cells transduced with this construct based on findings from the previous experiments.

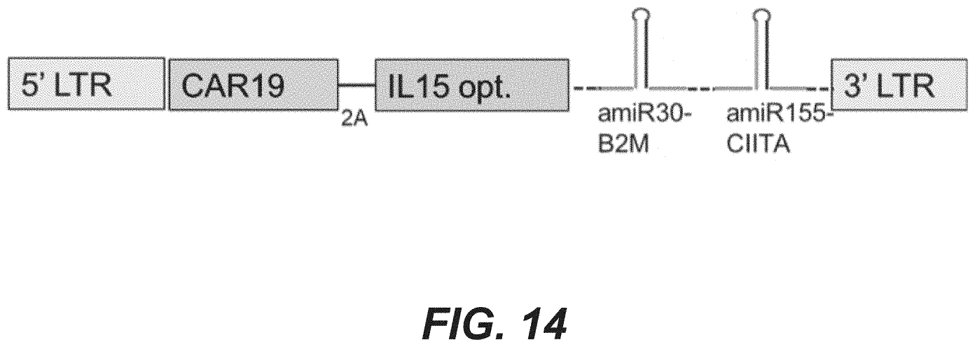

The efficacy of HLA class I and II knockdown mediated by this double knockdown construct (FIG. 14) is evaluated in transduced NKT cells. An IL15 ELISA is also performed to determine whether the presence of the additional amiR-shRNA impacts IL15 expression or secretion. Additionally, the anti-tumor activity of NKT cells expressing this construct is also evaluated in relevant in vitro and in vivo models.

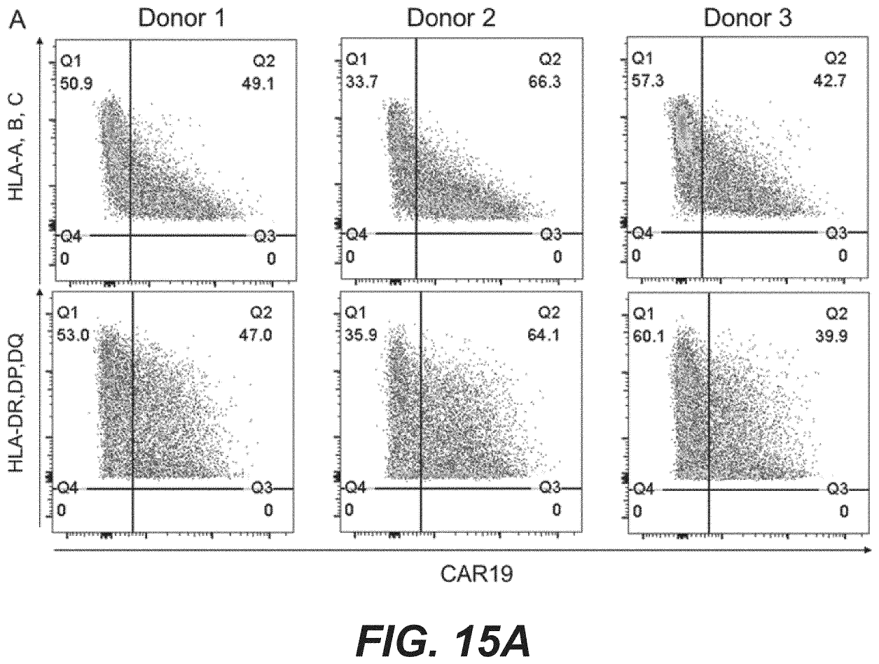

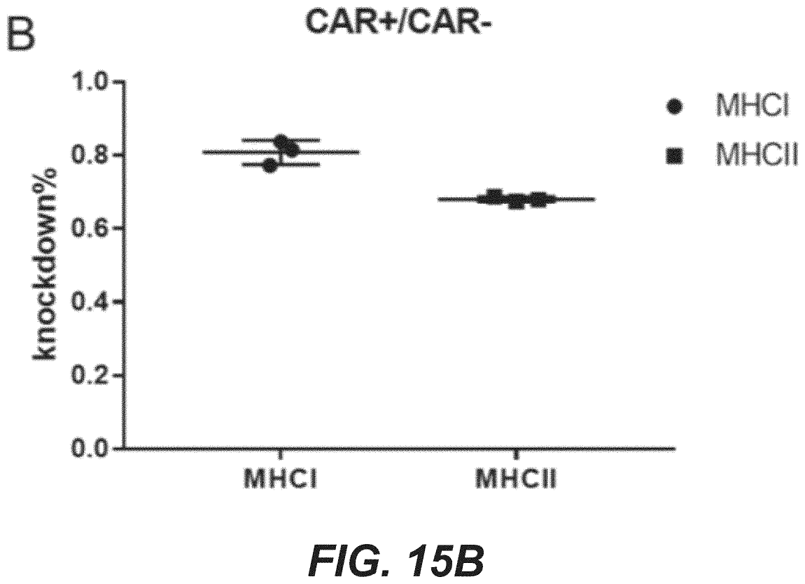

FIG. 15 shows that CAR19.opti-IL15 double knockdown construct mediates effective HLA class I and II knockdown in NKTs from three donors 10 days post-transduction. NKT cells are transduced with CAR19 construct shown in FIG. 14. CAR, HLA-A,B,C, and HLA-DR,DP,DQ expression are evaluated 10 days post-transduction. Knockdown percentage results for the three donors (BL #81, 82, 83) are summarized in FIG. 15, panel B. Table 10 below presents the data corresponding to FIG. 15B.

TABLE 10

| | Donor 1 | Donor 2 | Donor 3 |
|---|---|---|---|
| MHC class I KD % | 0.814455 | 0.836503 | 0.772071 |
| MHC class II KD % | 0.67364 | 0.686854 | 0.678659 |

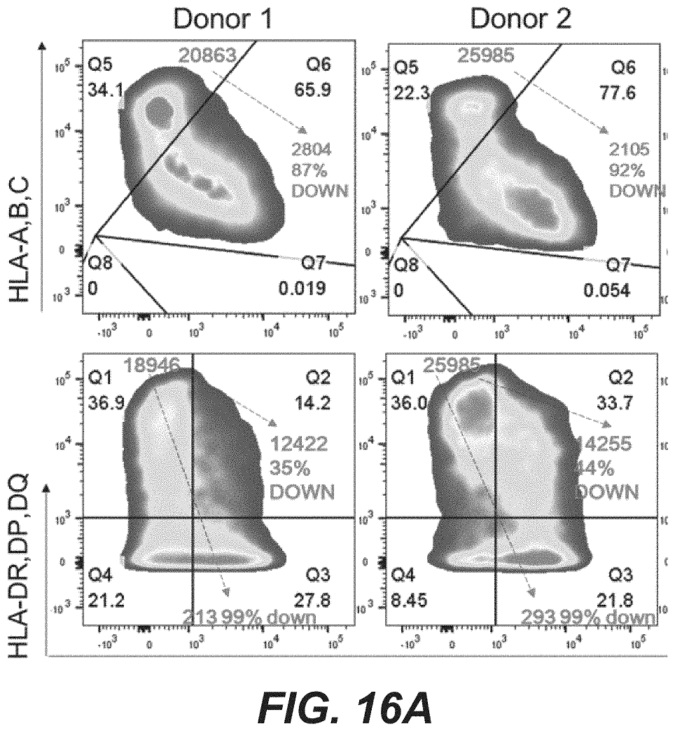

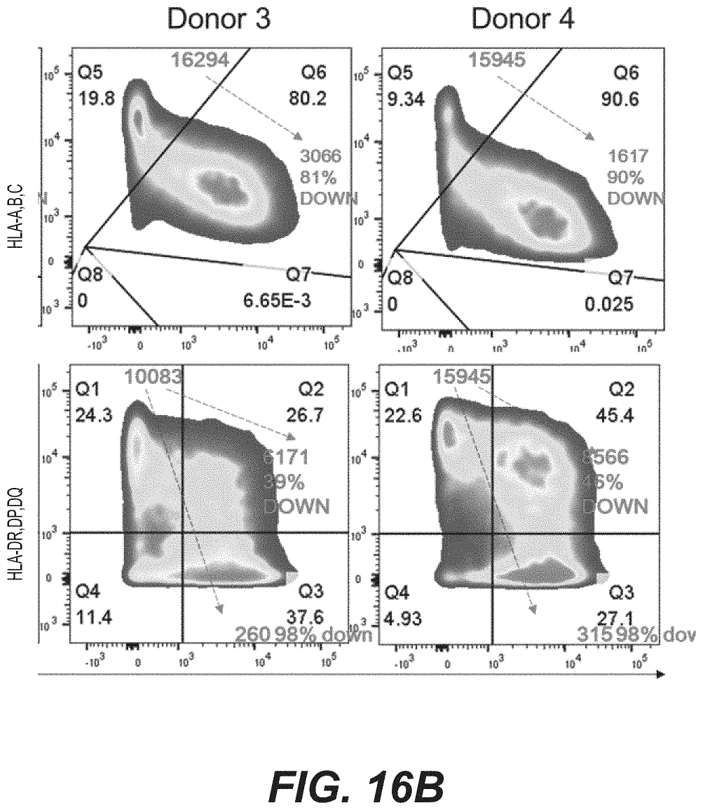

FIG. 16 shows that CAR19.opti-IL15 double knockdown construct mediates effective HLA class I and II knockdown in NKTs from four healthy donors at day 19 post-transduction.

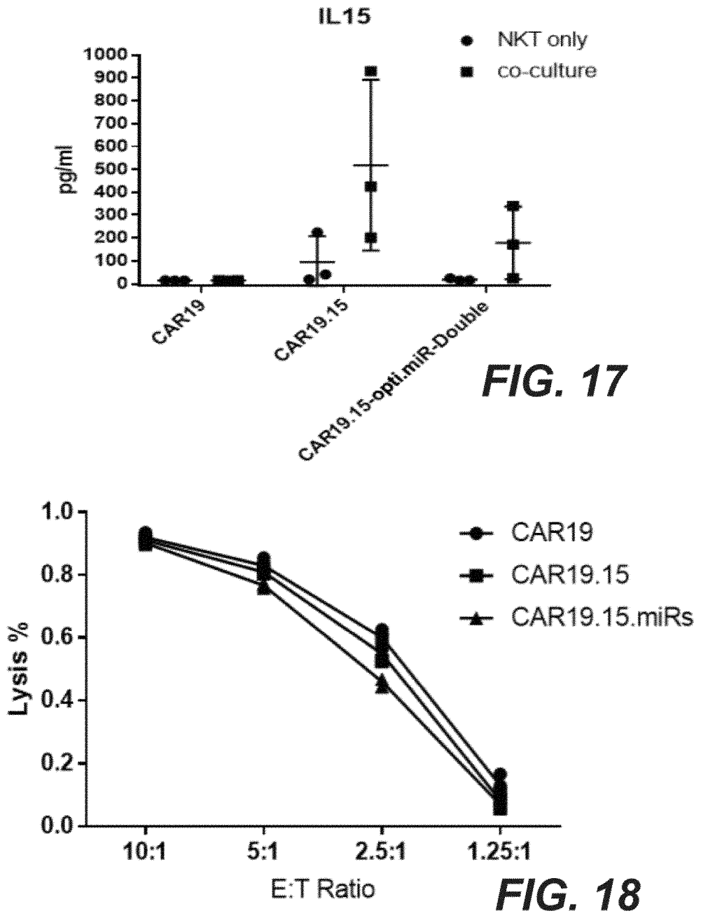

FIG. 17 shows that L15 secretion remains lower in NKT cells expressing CAR19.opti-IL15 double knockdown construct versus the original CAR19.15 construct. The NKT cells are transduced with the indicated constructs or non-transduced and either cultured alone or co-cultured with CD19+ Raji lymphoma cells for 48 hours. The culture supernatant is then processed using the BioLegend ELISA MAX™ Deluxe Set Human IL-15 kit (BioLegend #435104) to detect IL15 secretion. N=3 donors (BL #81, 82, 83). Table 11 below presents data corresponding to FIG. 17.

TABLE 11

| | NKT only | | | Lymphoma cell co-culture | | |
|---|---|---|---|---|---|---|
| CAR19 | 14.93776 | 15.11065 | 15.47372 | 14.29806 | 15.52559 | 14.64385 |
| CAR19.15 | 40.88866 | 224.6369 | 19.31189 | 424.5678 | 929.6162 | 200.4495 |
| CAR19.opti15.miR double | 15.73306 | 24.44675 | 15.16252 | 170.2974 | 339.6611 | 24.03181 |

FIG. 18 indicates that NKT cells transduced with CAR19.opti-IL15 double knock-down construct show similar level of in vitro cytotoxicity against CD19-positive target cells compared with CAR19 and CAR19.IL15 NKT cells. The NKT cells are transduced with indicated constructs and co-cultured for six hours with CD19+ Raji lymphoma cells engineered to express high levels of firefly luciferase at specified effector-to-target ratios. Luciferin is added at the conclusion of the assay for detection of bioluminescence. Table 12 below presents data corresponding to FIG. 18.

TABLE 12

| | 10 to 1 | | | 5 to 1 | | |
|---|---|---|---|---|---|---|
| CAR19 | 0.935568 | 0.917244 | 0.909342 | 0.854247 | 0.814848 | 0.821503 |
| CAR19.15 | 0.917881 | 0.910696 | 0.905255 | 0.823626 | 0.803791 | 0.800438 |
| CAR19.opti15.miR double | 0.905981 | 0.901736 | 0.897573 | 0.759216 | 0.773896 | 0.767962 |
| | 2.5 to 1 | | | 1.25 to 1 | | |
| CAR19 | 0.626282 | 0.56297 | 0.61185 | 0.129573 | 0.094069 | 0.166452 |
| CAR19.15 | 0.593768 | 0.52453 | 0.5276 | 0.105881 | 0.086567 | 0.056699 |
| CAR19.opti15.miR double | 0.444 | 0.467793 | 0.469963 | 0.06229 | 0.072074 | 0.073339 |

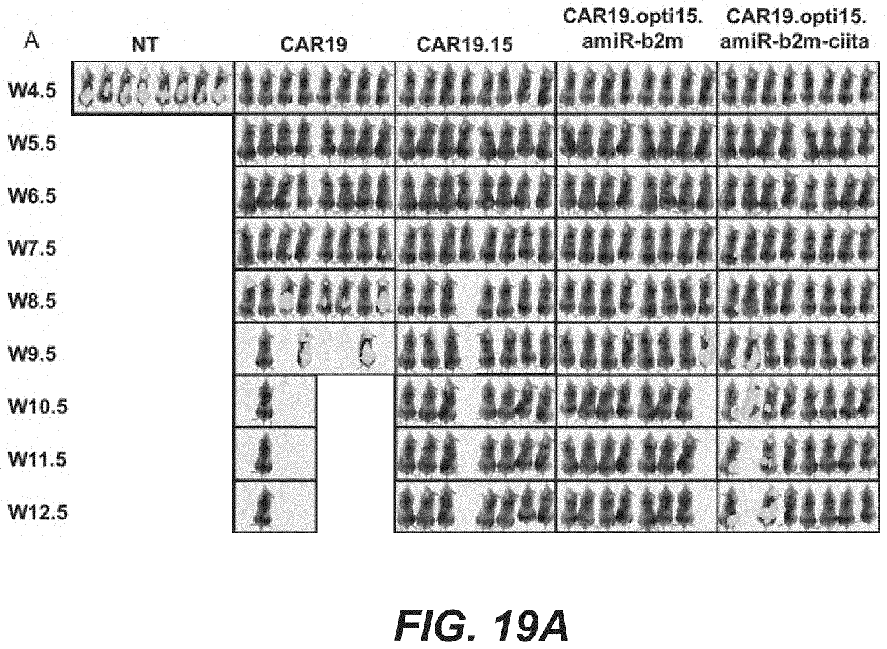

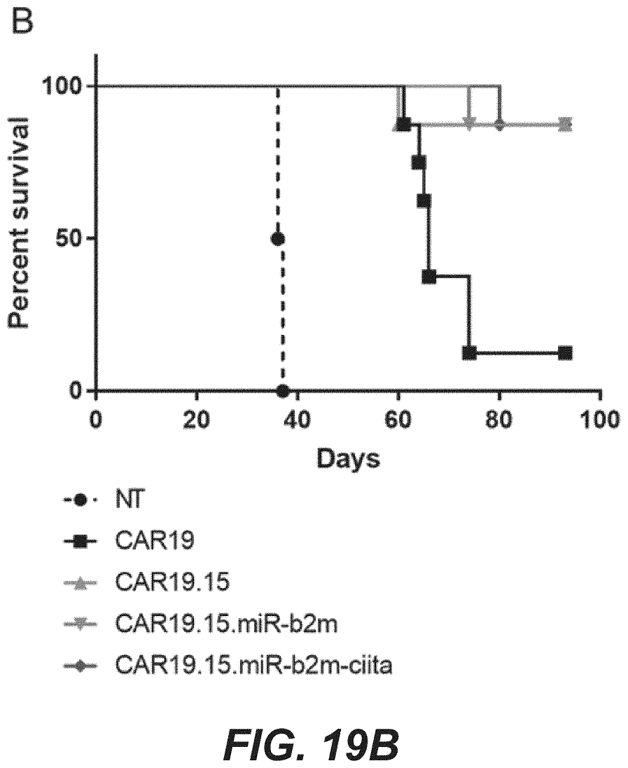

FIG. 19 demonstrates that NKT cells transduced with CAR19.opti-IL15 double knockdown construct control CD19+ tumors in vivo and promote survival of NSG mice comparably to CAR19.15 NKTs. NSG mice are injected intravenously with 2×10⁵ firefly luciferase-positive Daudi lymphoma cells on day 0 followed by intravenous injection of 5×10⁶ NKTs transduced with indicated constructs or no construct (non-transduced, NT) on day 3. Just prior to imaging, each mouse receive 100 μL luciferin at 30 mg/mL via intraperitoneal injection and are imaged under a bioluminescent channel. Bioluminescent counts scale 600-30,000. Panel B is the Kaplan Meier survival curve for mice shown in Panel A. Table 13 below presents data corresponding to FIG. 19B.

TABLE 13

| | Experimental Day (Death) | | | | | | | |
|---|---|---|---|---|---|---|---|---|
| | 37 | 37 | 37 | 37 | 36 | 36 | 36 | 36 |
| NT | 1 | 1 | 1 | 1 | 1 | 1 | 1 | 1 |
| CAR19 | | | | | | | | |
| CAR19.15 | | | | | | | | |
| CAR19.15.miR-B2M | | | | | | | | |
| CAR19.15.miR-B2M-CIITA | | | | | | | | |
| | 64 | 93 | 65 | 74 | 66 | 66 | 74 | 61 |
| NT | | | | | | | | |
| CAR19 | 1 | 0 | 1 | 1 | 1 | 1 | 1 | 1 |
| CAR19.15 | | | | | | | | |
| CAR19.15.miR-B2M | | | | | | | | |
| CAR19.15.miR-B2M-CIITA | | | | | | | | |
| | 93 | 93 | 93 | 60 | 93 | 93 | 93 | 93 |
| NT | | | | | | | | |
| CAR19 | | | | | | | | |
| CAR19.15 | 0 | 0 | 0 | 1 | 0 | 0 | 0 | 0 |
| CAR19.15.miR-B2M | | | | | | | | |
| CAR19.15.miR-B2M-CIITA | | | | | | | | |
| | 93 | 93 | 93 | 93 | 93 | 93 | 93 | 74 |
| NT | | | | | | | | |
| CAR19 | | | | | | | | |
| CAR19.15 | | | | | | | | |
| CAR19.15.miR-B2M | 0 | 0 | 0 | 0 | 0 | 0 | 0 | 1 |
| CAR19.15.miR-B2M-CIITA | | | | | | | | |
| | 93 | 80 | 93 | 93 | 93 | 93 | 93 | 93 |
| NT | | | | | | | | |
| CAR19 | | | | | | | | |
| CAR19.15 | | | | | | | | |
| CAR19.15.miR-B2M | | | | | | | | |
| CAR19.15.miR-B2M-CIITA | 0 | 1 | 0 | 0 | 0 | 0 | 0 | 0 |

Taken together, these experiments demonstrate that CAR19.opti-IL15 double knockdown construct mediates effective HLA class I and II knockdown in NKTs. NKTs transduced with CAR19.opti-IL15 double knockdown construct show similar level of in vitro cytotoxicity against CD19-positive target cells compared with CAR19 and CAR19.IL15 NKTs. NKTs transduced with CAR19.opti-IL15 double knockdown construct control CD19+ tumors in vivo and promote survival of NSG mice comparably to CAR19.15 NKTs. IL15 secretion remains lower in NKTs expressing CAR19.opti-IL15 double knockdown construct versus original CAR19.15.

### Example 6: Replacing IL15 Signal Peptide with IL2 Signal Peptide to Boost IL15 Secretion from NKTS Expressing Double Knockdown Construct

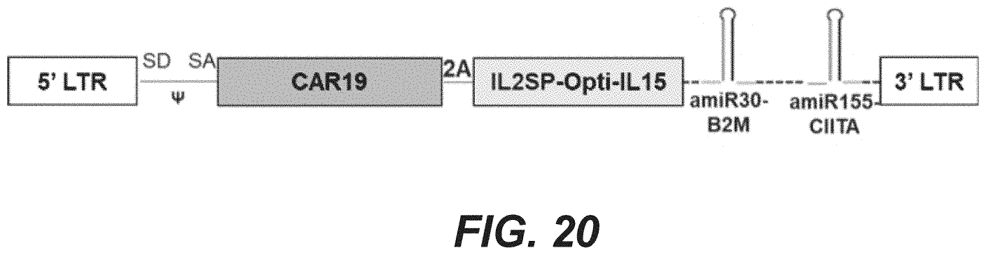

In order to enhance secretion of IL15 by NKT cells expressing the double knock-down construct, the IL15 signal peptide is replaced with the IL2 signal peptide, which is commonly used to mediate secretion of fusion proteins (FIG. 20). IL15 secretion by NKTs expressing the modified construct versus the original construct is compared. Anti-tumor activity experiments in NSG mice are also performed to evaluate any impact on in vivo function.

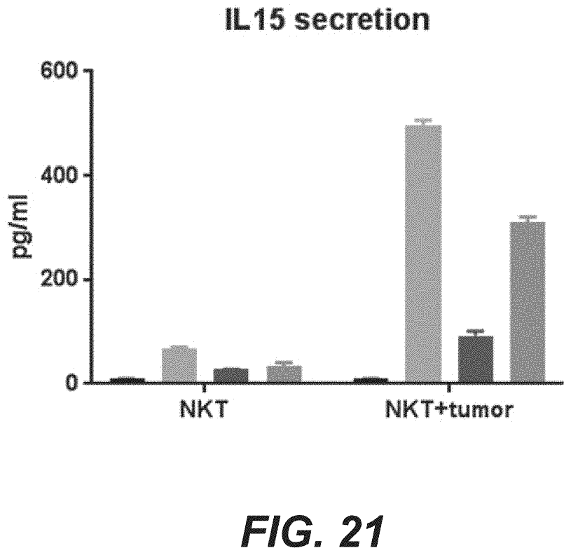

FIG. 21 indicates that the IL2 signal peptide boosts IL15 secretion by NKT cells expressing double knockdown construct. NKT cells are transduced with the indicated constructs or non-transduced and either cultured alone or co-cultured with CD19+ Raji lymphoma cells for 48 hours. The culture supernatant is then processed using the BioLegend ELISA MAX™ Deluxe Set Human IL-15 kit (BioLegend #435104) to detect IL15 secretion. Table 14 presents the data corresponding to FIG. 21.

TABLE 14

| | NKT cells | | | NKT + tumor | | |
|---|---|---|---|---|---|---|
| CAR19 | 9.390671 | 9.026692 | 9.053653 | 8.784039 | 9.148018 | 9.458075 |
| CAR19.15 | 66.54894 | 69.09679 | 70.8358 | 485.5433 | 494.4136 | 506.5732 |
| CAR19.15.miRs | 28.30413 | 27.58965 | 27.8997 | 102.6099 | 84.01995 | 89.74926 |
| CAR19.IL2SP-15.miRs | 26.0124 | 38.05069 | 37.78107 | 314.9447 | 317.7892 | 298.66 |

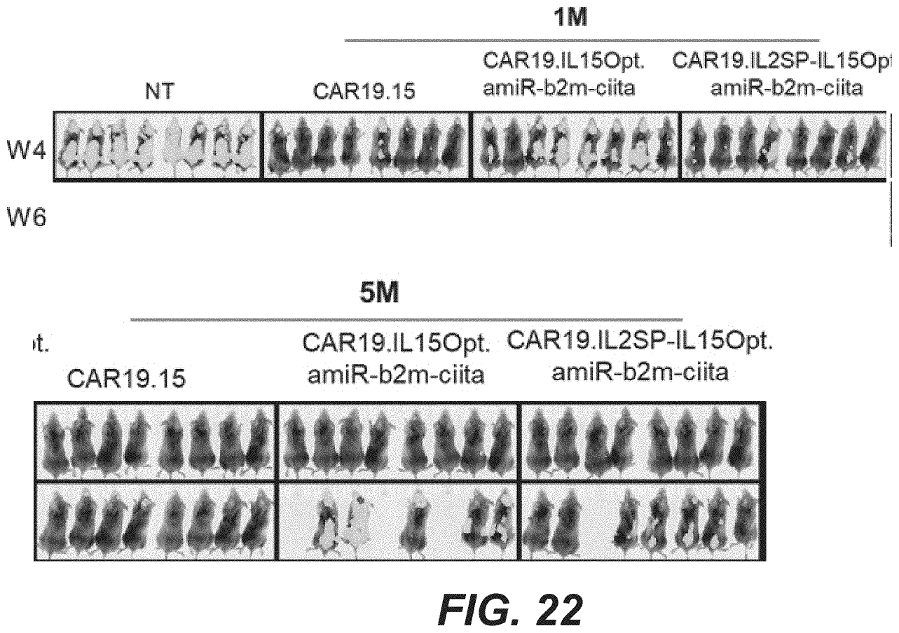

FIG. 22 shows the in vivo evaluation of NKTs expressing IL2SP-opti IL15 CAR19 construct with double amiR knockdown. NSG mice are injected intravenously with 2×10⁵ firefly luciferase-positive Daudi lymphoma cells on day 0 followed by intravenous injection of 1×10⁶ or 5×10⁶ NKTs transduced with indicated constructs or no construct (non-transduced, NT) on day 4. Just prior to imaging, each mouse receives 100 μL luciferin at 30 mg/mL via intraperitoneal injection and are imaged under a bioluminescent channel. Bioluminescent counts scale 600-30,000.

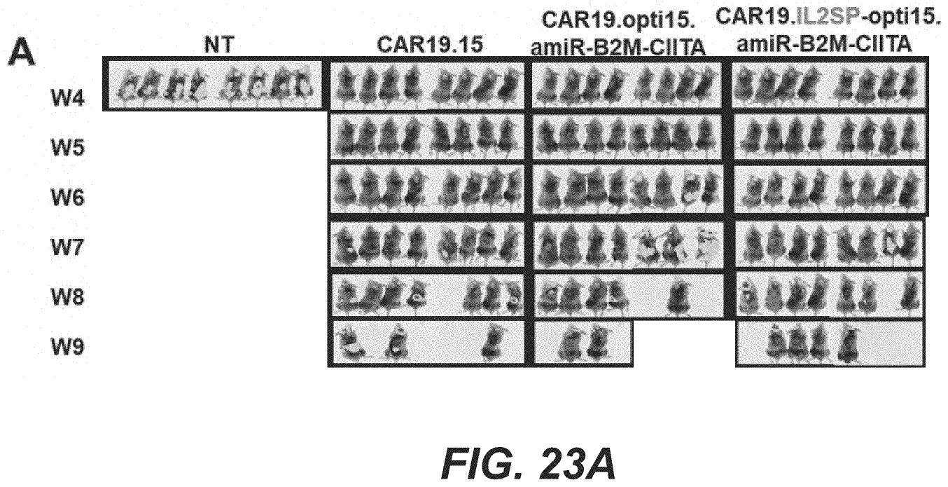

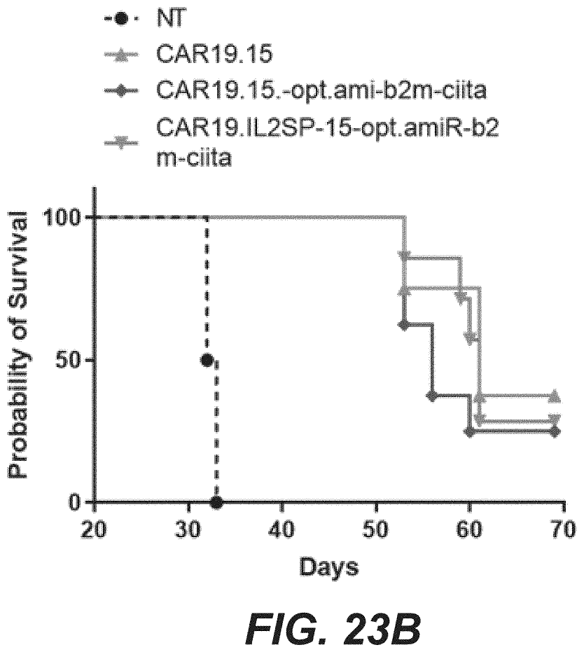

FIG. 23 indicates that IL2SP appears to delay tumor progression in NSG mice albeit without extending survival of mice treated with CAR NKTs expressing double knockdown construct. NSG mice are injected intravenously with 2×10⁵ firefly luciferase-positive Daudi lymphoma cells on day 0 followed by intravenous injection of 5×10⁶ NKTs transduced with indicated constructs or no construct (non-transduced, NT) on day 3. Just prior to imaging, each mouse receives 100 μL luciferin at 30 mg/mL via intraperitoneal injection and are imaged under a bioluminescent channel. Bioluminescent counts scale 2000-30,000. Panel B is the Kaplan Meier survival curve for mice shown in Panel A. Table 15 below presents the data corresponding to FIG. 23B.

TABLE 15

| | 33 | 33 | 33 | 33 | 32 | 32 | 32 | 32 |
|---|---|---|---|---|---|---|---|---|
| NT | 1 | 1 | 1 | 1 | 1 | 1 | 1 | 1 |
| CAR19.15 | | | | | | | | |
| CAR19.15-opt-amiR-B2M- | | | | | | | | |
| CIITA | | | | | | | | |
| CAR19.IL2SP-15-opti.amiR- | | | | | | | | |
| B2M-CIITA | | | | | | | | |
| | 61 | 65 | 61 | 65 | 53 | 65 | 61 | 53 |
| NT | | | | | | | | |
| CAR19.15 | 1 | 0 | 1 | 0 | 1 | 0 | 1 | 1 |
| CAR19.15-opt-amiR-B2M- | | | | | | | | |
| CIITA | | | | | | | | |
| CAR19.IL2SP-15-opti.amiR- | | | | | | | | |
| B2M-CIITA | | | | | | | | |
| | 56 | 65 | 65 | 56 | 53 | 60 | 53 | 53 |
| NT | | | | | | | | |
| CAR19.15 | | | | | | | | |
| CAR19.15-opt-amiR-B2M- | 1 | 0 | 0 | 1 | 1 | 1 | 1 | 1 |
| CIITA | | | | | | | | |
| CAR19.IL2SP-15-opti.amiR- | | | | | | | | |
| B2M-CIITA | | | | | | | | |
| | 65 | 65 | 65 | 60 | 59 | 53 | 65 | |
| NT | | | | | | | | |
| CAR19.15 | | | | | | | | |
| CAR19.15-opt-amiR-B2M- | | | | | | | | |
| CIITA | | | | | | | | |
| CAR19.IL2SP-15-opti.amiR- | 0 | 0 | 0 | 1 | 1 | 1 | 0 | |
| B2M-CIITA | | | | | | | | |

Taken together, these experiments show that IL2 signal peptide boosts IL15 secretion by NKTs expressing double knockdown construct. IL2SP may delay tumor progression in mice treated with CAR NKTs expressing double knockdown construct.

### Example 7: Evaluating Allogenicity of NKTS Expressing Double Knockdown Construct Via Mixed Lymphocyte Reactions (MLR)

The ultimate goal of knocking down HLA class I and II expression is to reduce the allogenicity of transduced NKTs, thereby preventing or delaying rejection and increasing the therapeutic time window for these cells within an allogeneic patient.

To determine how HLA knock-down mediated by the amiR construct impacts NKT allogenicity, several mixed lymphocyte reactions (MLRs) are performed by co-culturing CAR19.IL2SP-opti15 double knockdown (CAR19.IL2SP-opti15.amiR-B2M-amiR-CIITA) NKTs with HLA-mismatched NK cells, T cells, or PBMCs. At multiple time-points during co-culture, NKT cell numbers, CAR expression, and HLA expression are evaluated to determine whether the NKTs are able to persist in the presence of allogeneic immune cells. In parallel, the same co-cultures are performed using CAR19.IL2SP-opti15 NKTs with scrambled shRNA sequences in place of B2M and CIITA shRNA sequences (CAR19.IL2SP-opti15.amiR-SCR-amiR-SCR), as well as NKTs with B2M and CIITA knocked out (mediated by specific guide RNAs via CRISPR/Cas9).

In addition, several in vivo MLR rejection assays are performed, including allogeneic T cell and PBMC rejection. In these experiments, HLA-mismatched recipient T cells or PBMCs are infused into NSG mice (MHC null in the case of PBMCs) followed four days later by donor NKT cells expressing the amiR double knockdown construct with B2M/CIITA shRNAs or scrambled sequence shRNAs. The T cell rejection model is also evaluated in the context of mice with CD19+ Daudi lymphoma tumors.

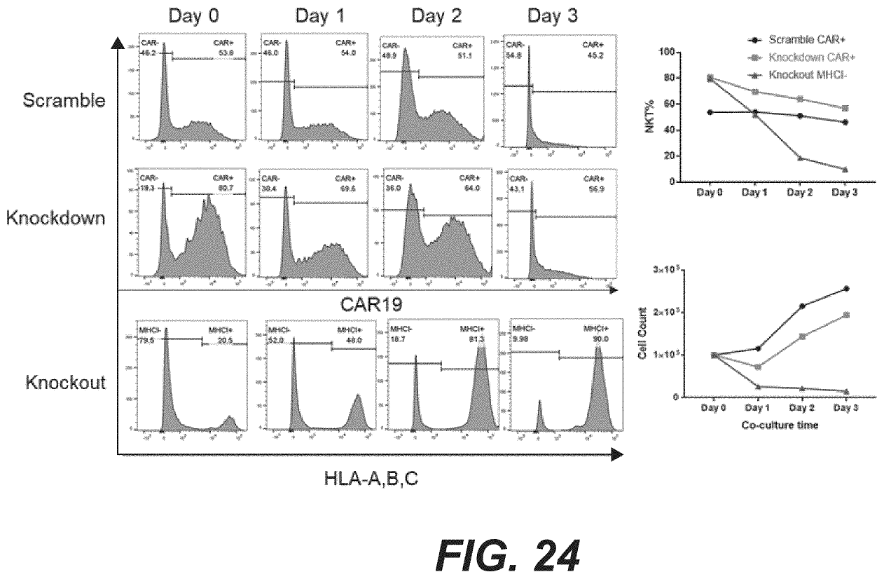

As shown in FIG. 24, NKTs expressing the B2M/CIITA double knockdown construct persist in the presence of allogeneic NK cells while double knock-out leaves NKTs vulnerable to NK cell killing in the in vitro MLR. Recipient NK cells (HLA-A2+) are isolated using the NK cell isolation kit (Miltenyi Biotech) and co-cultured with donor NKTs (HLA-A2−) at a 1:1 ratio for three days. NKTs are transduced with 1) CAR19.15 containing two scrambled shRNA sequences in place of B2M and CIITA (CAR19.IL2SP-opti15.amiR-SCR-amiR-SCR, scramble), 2) CAR19.15 with amiR-embedded B2M and CIITA shRNA sequences (CAR19.IL2SP-opti15.amiR-B2M-amiR-CIITA, knockdown), 3) NKTs with B2M/CIITA double knockout. NKTs are evaluated by flow cytometry daily for CAR and HLA expression, gated on HLA I-cells. Table 16 below presents the data corresponding to FIG. 24.

TABLE 16

| NKT % | Scramble | KD | KO |
|---|---|---|---|
| Day 0 | 53.8 | 80.7 | 79.5 |
| Day 1 | 54 | 69.6 | 52 |
| Day 2 | 51.1 | 64 | 18.7 |
| Day 3 | 46.2 | 56.9 | 9.98 |
| NK count | Scramble | KD | KO |
| Day 0 | 100000 | 100000 | 100000 |
| Day 1 | 115266.3 | 72228.1 | 25968.8 |
| Day 2 | 215024.7 | 143390.7 | 21524.45 |
| Day 3 | 256026.4 | 194198.6 | 14602 |

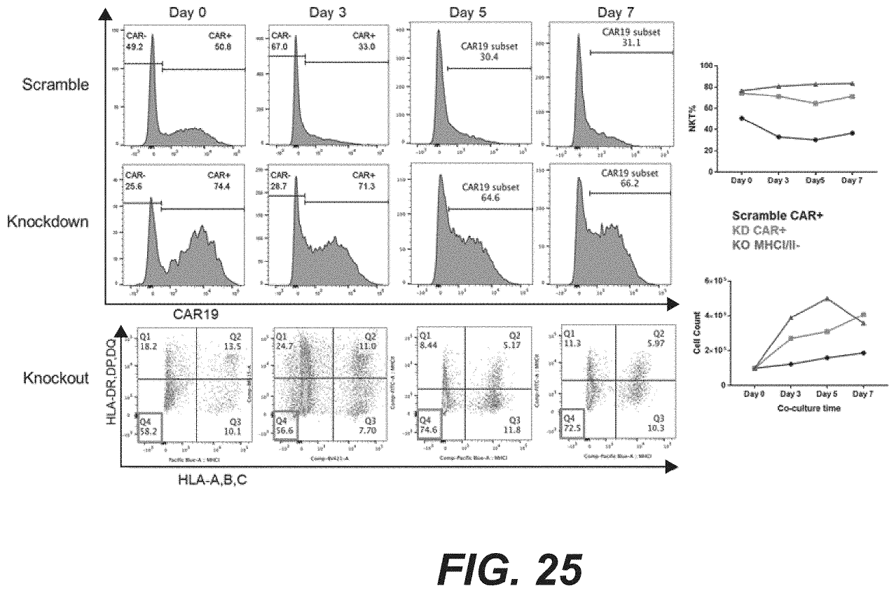

As shown in FIG. 25, NKTs expressing the B2M/CIITA double knockdown construct resist rejection by allogeneic T cells compared to NKTs carrying scrambled shRNA control construct in the in vitro MLR. Pan T cells are isolated from recipient PBMCs using the naive pan T cell isolation kit, human (Miltenyi Biotech. Recipient T cells (HLA-A2+) are co-cultured with donor NKTs (HLA-A2−) at a 2:1 (T:NKT) ratio for seven days. NKTs are transduced with 1) CAR19.15 scrambled shRNA control, 2) CAR19.15 with double knockdown, 3) NKTs with B2M/CIITA double knockout. NKTs are evaluated by flow cytometry every 2-3 days. Tables 17 and 18 below present the data corresponding to FIG. 25.

TABLE 17

| NKT % | Scramble | KD | KO |
|---|---|---|---|
| Day 0 | 50.8 | 74.4 | 76.7 |
| Day 3 | 33 | 71.3 | 80.9 |
| Day 5 | 30.4 | 64.6 | 82.8 |
| Day 7 | 36.6 | 71.2 | 83.5 |

TABLE 18

| T count | Scramble | KD | KO |
|---|---|---|---|
| Day 0 | 100000 | 100000 | 100000 |
| Day 3 | 123337.5 | 271054.1 | 390310.1 |
| Day 5 | 159463.8 | 310390.1 | 499668.2 |
| Day 7 | 186958.7 | 407170 | 358134.8 |

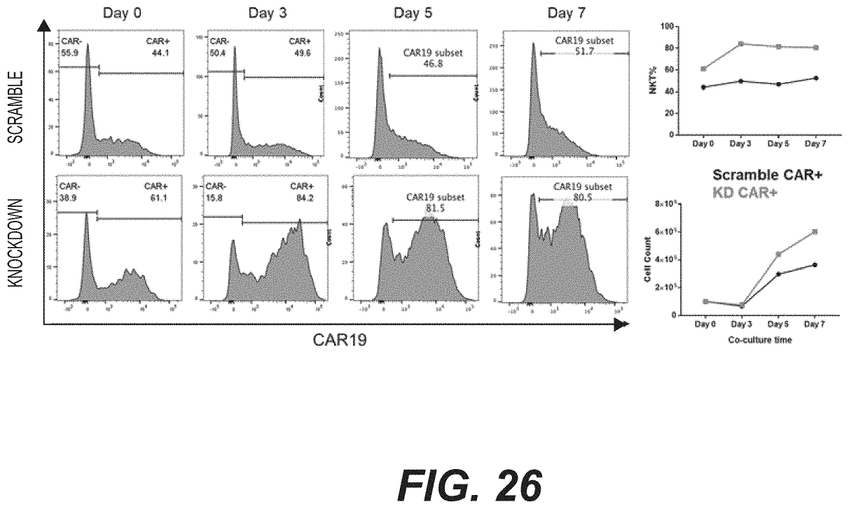

As shown in FIG. 26, NKTs expressing the B2M/CIITA double knockdown construct resist rejection by allogeneic PBMCs compared to NKTs carrying scrambled shRNA control construct in the in vitro MLR. Recipient PBMCs (HLA-A2+) are co-cultured with donor NKTs (HLA-A2−) at a 10:1 (PBMC:NKT) ratio for seven days. NKTs are transduced with 1) CAR19.15 with scrambled shRNA control, or 2) CAR19.15 with double knockdown. NKT cells are evaluated by flow cytometry every 2-3 days. Tables 19 and 20 below present the data corresponding to FIG. 26.

TABLE 19

| NKT % | Scramble | KD |
|---|---|---|
| Day 0 | 44.1 | 61.1 |
| Day 3 | 49.6 | 84.2 |
| Day 5 | 46.8 | 81.5 |
| Day 7 | 52.5 | 80.8 |

TABLE 20

| PBMC count | Scramble | KD |
|---|---|---|
| Day 0 | 100000 | 100000 |
| Day 3 | 66154.5 | 74733.23 |
| Day 5 | 298614 | 439500.2 |
| Day 7 | 363283.2 | 603981.6 |

As shown in FIG. 27, NKTs expressing the B2M/CIITA double knockdown construct resist killing by allogeneic NK cells while double knockout leaves NKTs vulnerable to NK cell killing in the in vitro MLR. Recipient NK cells (HLA-A2+) are isolated using the NK cell isolation kit (Miltenyi Biotech) and co-cultured after isolation with donor NKTs (HLA-A2−) at a 2:1 (NK:NKT) ratio for two days. NKTs are transduced with 1) CAR19.IL2SP-opti15 with scrambled shRNA sequences (Scr), 2) CAR19.IL2SP-opti15 with double knockdown (KD), 3) CAR19.IL2SP-opti15 with double knockout (KO). A) Representative flow plots showing total frequency of donor NKT cells on day 0 and day 2 of co-culture. Absolute cell counts of B) donor NKT cells and C) recipient NK cells on day 0 and day 2 of co-culture. All data denote mean±s.d., three unique donor-recipient pairs are used. P values are determined using two-way ANOVA with Sidak's correction for multiple comparisons and nonsignificant (P>0.05) values are not shown. P values are determined using the two-tailed, paired Student's t-test. Table 21 below presents the data corresponding to FIG. 27A. Table 22 below presents the data corresponding to FIG. 27B.

TABLE 21

| | Scramble | | | KO | | | KD | | |
|---|---|---|---|---|---|---|---|---|---|
| Day 0 | 53404.05 | 58479.33 | 55945.89 | 37043.16 | 65688 | 53830.98 | 37441.95 | 57170.4 | 55380.78 |
| Day 2 | 172533.1 | 111520.1 | 178017.8 | 21791.27 | 31399.89 | 23076 | 107980.6 | 157472.7 | 94897.83 |

TABLE 22

| | Scramble | | | KO | | | KD | | |
|---|---|---|---|---|---|---|---|---|---|
| Day 0 | 138600 | 114660 | 144060 | 157920 | 113190 | 143220 | 136920 | 109200 | 128520 |
| Day 2 | 212940 | 259560 | 173880 | 429580 | 421400 | 442080 | 243100 | 192525 | 231200 |

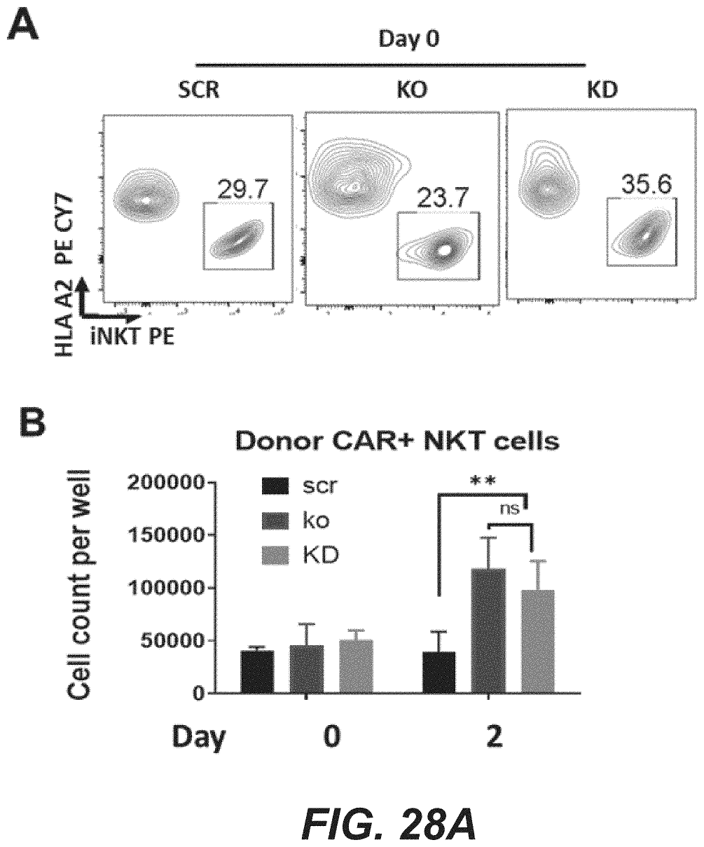

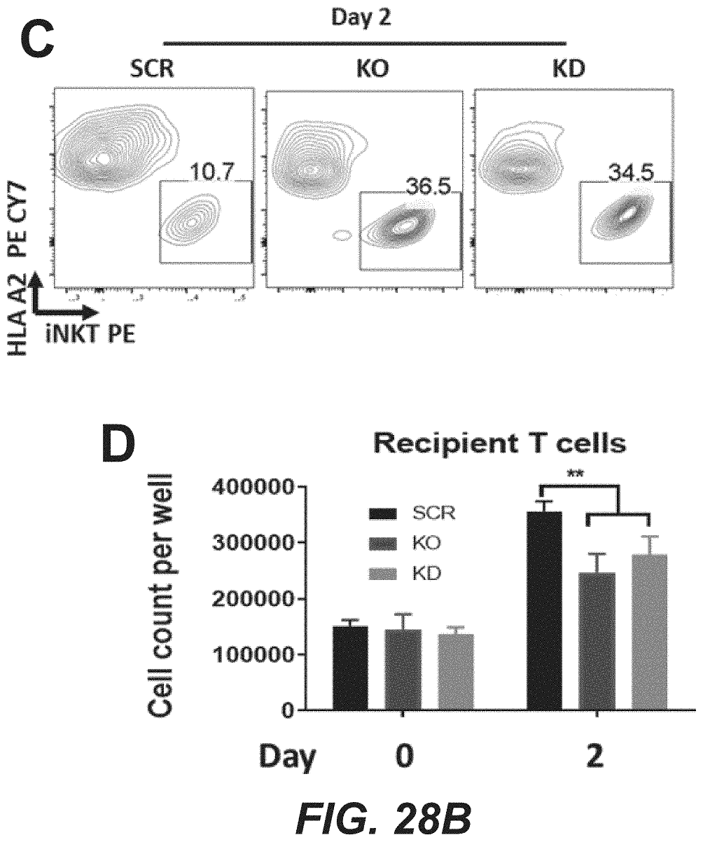

As shown in FIG. 28, NKTs expressing the B2M/CIITA double knockdown construct resist rejection by allogeneic T cells compared to NKTs carrying scrambled shRNA control construct in the in vitro MLR. Pan T cells are isolated from recipient PBMCs (HLA-A2+) using the naive pan T cell isolation kit, human (Miltenyi Biotech). Purified T cells are then stimulated with OKT3/αCD28 for 24 hours, in vitro expanded for 5-10 days, and co-cultured with donor NKTs (HLA-A2−) at a 2:1 (T:NKT) ratio for two days. NKTs are transduced with 1) CAR19.IL2SP-opti15 with scrambled shRNA sequences (Scr), 2) CAR19.IL2SP-opti15 with double knockdown (KD), 3) CAR19.IL2SP-opti15 with double knockout (KO). A) Representative flow plots showing total frequency of donor NKT cells on day 0 and day 2 of co-culture. Absolute cell counts of B) donor NKT cells and C) recipient T cells on day 2 of co-culture. All data denote mean±s.d., five unique donor-recipient pairs are used. P values are determined using two-way ANOVA with Sidak's correction for multiple comparisons and nonsignificant (P>0.05) values are not shown. Table 23 below presents the data corresponding to FIG. 28A. Table 24 below presents the data corresponding to FIG. 28B.

TABLE 23

| | Scramble | | | KO | | | KD | | |
|---|---|---|---|---|---|---|---|---|---|
| Day 0 | 37956.24 | 44163 | 40578.3 | 30635.85 | 38024.28 | 68668.32 | 44427.6 | 61103.7 | 46393.62 |
| Day 2 | 29457.12 | 28420.6 | 61302.36 | 136986.7 | 83705.39 | 133276.1 | 118738.6 | 108431.1 | 67381.2 |

TABLE 24

| | Scramble | | | KO | | | KD | | |
|---|---|---|---|---|---|---|---|---|---|
| Day 0 | 163800 | 144060 | 147630 | 168420 | 152250 | 115080 | 150570 | 127050 | 132510 |
| Day 2 | 372120 | 360400 | 336840 | 243600 | 282130 | 215100 | 254150 | 265625 | 315775 |

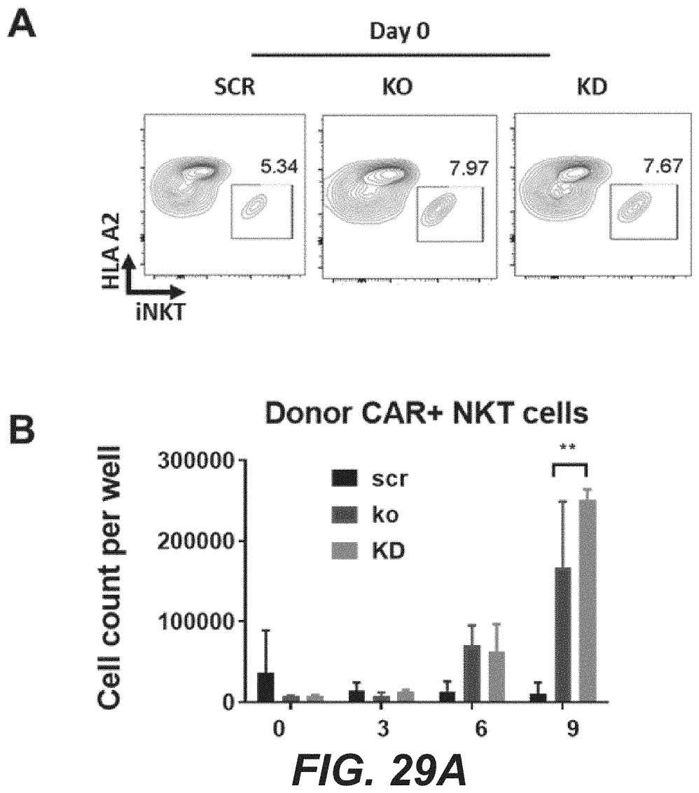

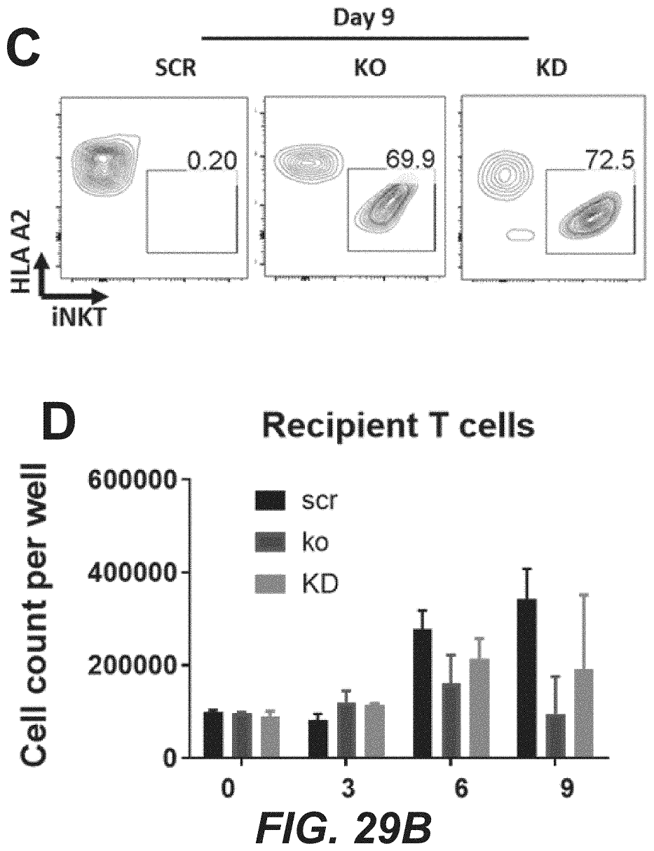

As shown in FIG. 29, NKTs expressing the B2M/CIITA double knockdown construct resist rejection by allogeneic PBMCs compared to NKTs carrying scrambled shRNA control construct in the in vitro MLR. Recipient whole PBMCs (HLA-A2+) are co-cultured with donor NKTs (HLA-A2−) at a 10:1 (PBMC:NKT) ratio for nine days. NKTs are transduced with 1) CAR19.IL2SP-opti15 with scrambled shRNA sequences (Scr), 2) CAR19.IL2SP-opti15 with double knockdown (KD), 3) CAR19.IL2SP-opti15 with double knockout (KO). A) Representative flow plots showing total frequency of donor NKT cells on day 0 and day 9 of co-culture. Absolute cell counts of B) donor NKT cells and C) recipient cells on days 0, 3, 6, and 9 of co-culture. All data denote mean±s.d., three unique donor-recipient pairs are used. P values are determined using two-way ANOVA with Sidak's correction for multiple comparisons and nonsignificant (P>0.05) values are not shown. P values are determined using the two-tailed, paired Student's t-test. Table 25 below presents the data corresponding to FIG. 29A. Table 26 below presents the data corresponding to FIG. 29B.

TABLE 25

| | Scramble | | | KO | | | KD | | |
|---|---|---|---|---|---|---|---|---|---|
| Day 0 | 9744.042 | 5721.276 | 5442.228 | 6684.579 | 8381.252 | 6684.579 | 8378.095 | 8040.461 | 8385.685 |
| Day 3 | 8452.44 | 9467.82 | 25905.24 | 4319.055 | 8339.31 | 11970 | 12051.59 | 11163.83 | 15810.39 |
| Day 6 | 28594.72 | 95.942 | 15760.5 | 72765 | 76387.5 | 73040 | 80778.88 | 55125.84 | 46390.4 |
| Day 9 | 41807.1 | 205.2 | 45659.08 | 161406.5 | 78926.4 | 141746 | 101154.2 | 264001.5 | 259461.2 |

TABLE 26

| | Scramble | | | KO | | | KD | | |
|---|---|---|---|---|---|---|---|---|---|
| Day 0 | 95040 | 102300 | 102740 | 100650 | 97680 | 100320 | 80410 | 99440 | 97350 |
| Day 3 | 106800 | 68220 | 90840 | 139950 | 128400 | 92250 | 115830 | 117045 | 110295 |
| Day 6 | 312200 | 340200 | 322105 | 178850 | 244300 | 115600 | 207100 | 242060 | 204400 |
| Day 9 | 443190 | 441900 | 505620 | 195320 | 273240 | 242800 | 266400 | 112200 | 317200 |

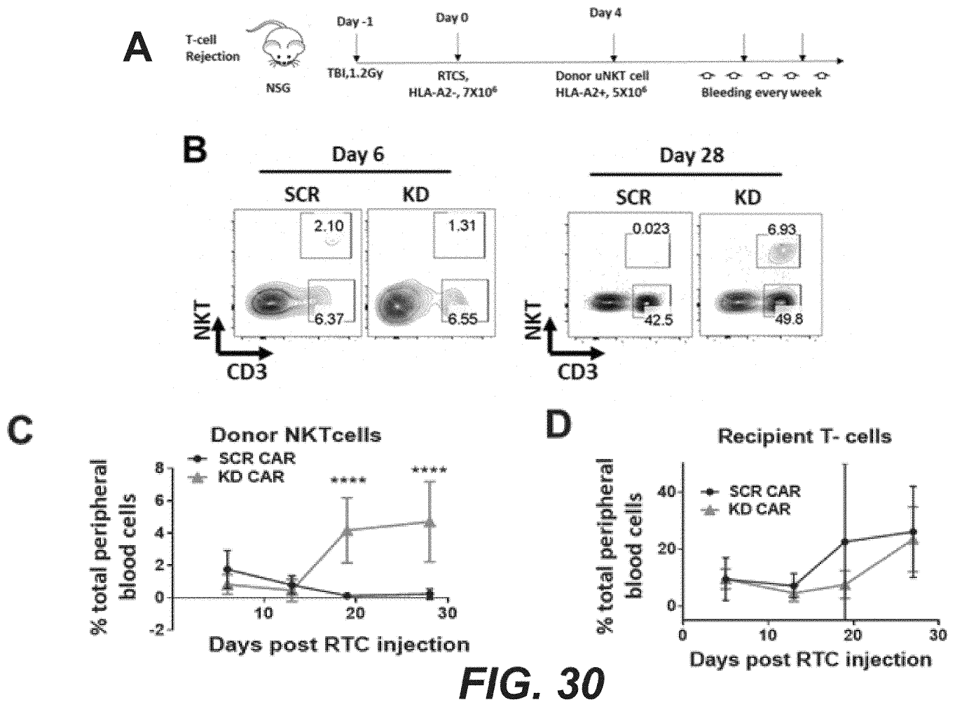

As shown in FIG. 30, NKTs expressing the B2M/CIITA double knockdown construct persist in vivo in the presence of allogeneic T cells compared to scrambled control NKTs in the in vivo T cell-mediated rejection model. A) NSG mice are irradiated at 1.2 Gy on day −1, and on the following day received 7×106 in vitro expanded human T-cells (day 5-10 post initial OKT3/αCD28 stimulation) from an HLA-A2-recipient. Four days later, mice received 2×10⁶ control construct (CAR19.IL2SP-opti15.amiR-SCR-amiR-SCR) or knockdown construct (CAR19.IL2SP-opti15.amiR-b2m-amiR-ciita) transduced NKTs from an HLA-A2+ donor intravenously. RTC=recipient T cells. B) Representative flow plot showing frequencies of donor HLA-A2+ Scr control or double KD NKT cells in peripheral blood on days 6 and 28. Frequency of C) donor HL-A2+ NKT cells and D) recipient HLA-A2-T-cells at specified time points. Data denote mean±SD with 7-8 mice per group. Table 27 below presents the data corresponding to FIG. 30, panel C. Table 28 below presents the data corresponding to FIG. 30, panel D.

TABLE 27

| | Scramble | | | | | | | |
|---|---|---|---|---|---|---|---|---|
| Day 6 | 0.89 | 1.1 | 4.06 | 2.13 | 2.1 | 4.62 | 1.59 | 0 |
| Day 13 | 0.13 | 0.49 | 0.078 | 0.076 | 0.39 | 0.067 | 0.53 | 0.69 |
| Day 19 | 0.44 | 0.27 | 0 | 0 | 0 | 0 | 0 | 0.092 |
| Day 28 | 0 | 0 | 0 | 0 | 0 | 0.024 | 0 | 0 |
| | KD | | | | | | | |
| Day 6 | 6.88 | 1.17 | 1.1 | 5.59 | 2.44 | 1.98 | 1.31 | 0.98 |
| Day 13 | 0.75 | 1.18 | 0.067 | 0.059 | 0.042 | 0.12 | 0.59 | 0.27 |
| Day 19 | 11 | 7.95 | 5.59 | 4.29 | 5.69 | 5.46 | 9.36 | 5.89 |
| Day 28 | 17.4 | 14.4 | 5.03 | 3.11 | 2.88 | 3.37 | 5.6 | 3.11 |

TABLE 28

| | Scramble | | | | | | | |
|---|---|---|---|---|---|---|---|---|
| Day 5 | 9.15 | 4.72 | 4.11 | 6.17 | 6.37 | 3.75 | 18.3 | 24 |
| Day 13 | 5.94 | 9.22 | 1.17 | 9.88 | 9.03 | 0.86 | 8.31 | 13.1 |
| Day 19 | 77.7 | 19.4 | 8.02 | 5.19 | 8.97 | 5.43 | 3.46 | 52.7 |
| Day 27 | 25.9 | 17 | 8.43 | 23.3 | 55.2 | 44 | 12 | 23 |
| | KD | | | | | | | |
| Day 5 | 11.2 | 6.77 | 7.51 | 10.2 | 15.6 | 12.9 | 6.55 | 5.85 |
| Day 13 | 5.12 | 8.98 | 5.37 | 0.68 | 1.02 | 2.56 | 7.32 | 5.89 |
| Day 19 | 8.59 | 4.53 | 5.9 | 1.14 | 17.8 | 8.92 | 6.88 | 7.01 |
| Day 27 | 13.4 | 11.2 | 33.1 | 23.5 | 16.7 | 38.7 | 14 | 37.3 |

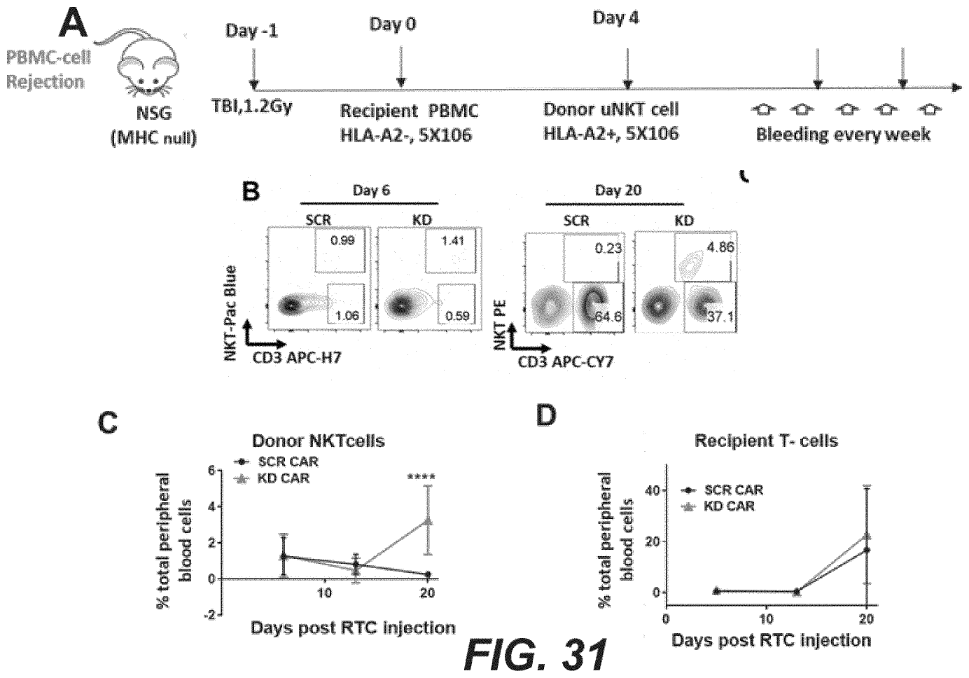

As shown in FIG. 31, NKTs expressing the B2M/CIITA double knockdown construct persist in vivo in the presence of allogeneic PBMCs compared to scrambled control NKTs in the in vivo PBMC-mediated rejection model. A) NSG)(MHC^{KO}) mice are irradiated at 1.2 Gy on day −1, and then received intravenously 5×10⁶ freshly isolated PBMC from an HLA-A2− recipient on day 0. Four days later, 5×10⁶ scrambled control or double knockdown transduced NKTs from an HLA-A2+ donor are administered intravenously. B) Representative flow plot showing frequencies of donor HLA-A2+ Scr control or double KD NKT cells in peripheral blood on days 6 and 20. Frequency of C) donor HL-A2+ NKT cells and D) recipient HLA-A2−T cells at specified time points. Data denote mean±SD with 7-8 mice per group. Table 29 below presents the data corresponding to FIG. 31, panel C. Table 30 below presents the data corresponding to FIG. 31, panel D.

TABLE 29

| | Scramble | | | | | | | |
|---|---|---|---|---|---|---|---|---|
| Day 6 | 3.5 | 1.38 | 1.77 | 0.99 | 0.39 | 1.03 | 0.61 | 0.31 |
| Day 13 | 0.038 | 0.059 | 0.26 | 0.065 | 0.031 | 0 | 0.12 | 0.077 |
| Day 20 | 0.12 | 0.085 | 0.23 | 0.69 | 0.2 | 0.2 | 1.02 | 0.98 |
| | KD | | | | | | | |
| Day 6 | 0.59 | 0.53 | 1.03 | 1.41 | 2.3 | 0.41 | 3.73 | 0.27 |
| Day 13 | 0.034 | 0.044 | 0.2 | 0.16 | 0.034 | 0.053 | 0.08 | 0.22 |
| Day 20 | 2.87 | 4.34 | 5.44 | 4.86 | 4.9 | 1.53 | 1.68 | 0.29 |

TABLE 30

| | Scramble | | | | | | | |
|---|---|---|---|---|---|---|---|---|
| Day 5 | 0.87 | 0.57 | 0.41 | 1.06 | 0 | 1.03 | 0.26 | 0.012 |
| Day 13 | 0.79 | 0.18 | 0.25 | 1.02 | 0.41 | 0.57 | 0.23 | 0.19 |
| Day 20 | 17.8 | 0.9 | 64.6 | 41.9 | 3.29 | 2.16 | 1.52 | 1.97 |
| | KD | | | | | | | |
| Day 5 | 0.15 | 0.43 | 1.2 | 0.59 | 0.57 | 0.091 | 1.69 | 2.96 |
| Day 13 | 0.068 | 0.22 | 0.23 | 0.2 | 0.3 | 0.25 | 0.4 | 0.62 |
| Day 20 | 1.71 | 26.6 | 50.4 | 37.1 | 15.6 | 3.86 | 42.6 | 4.21 |

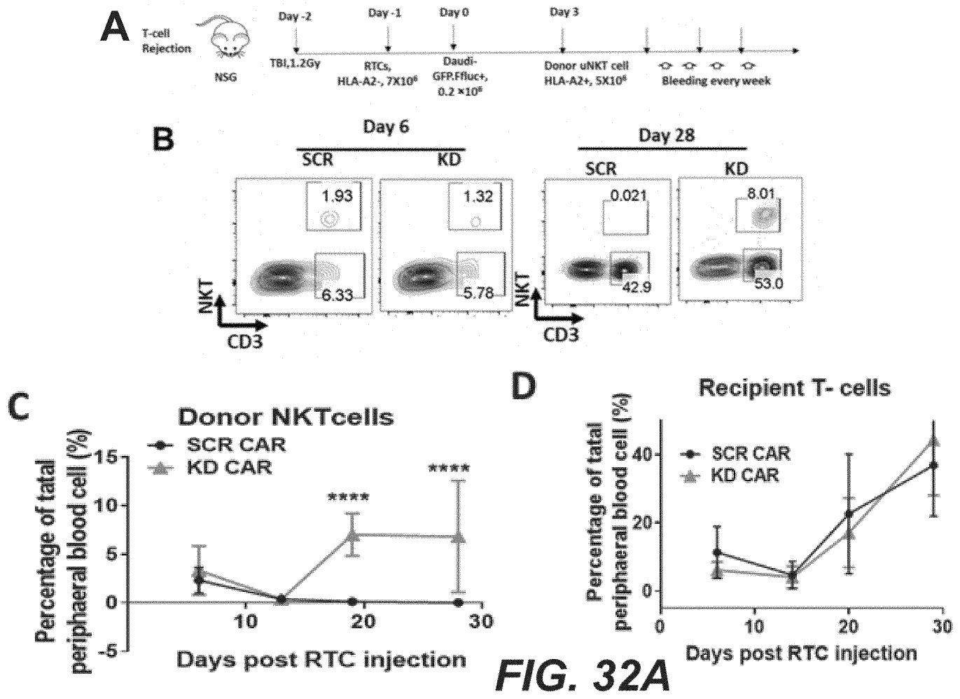

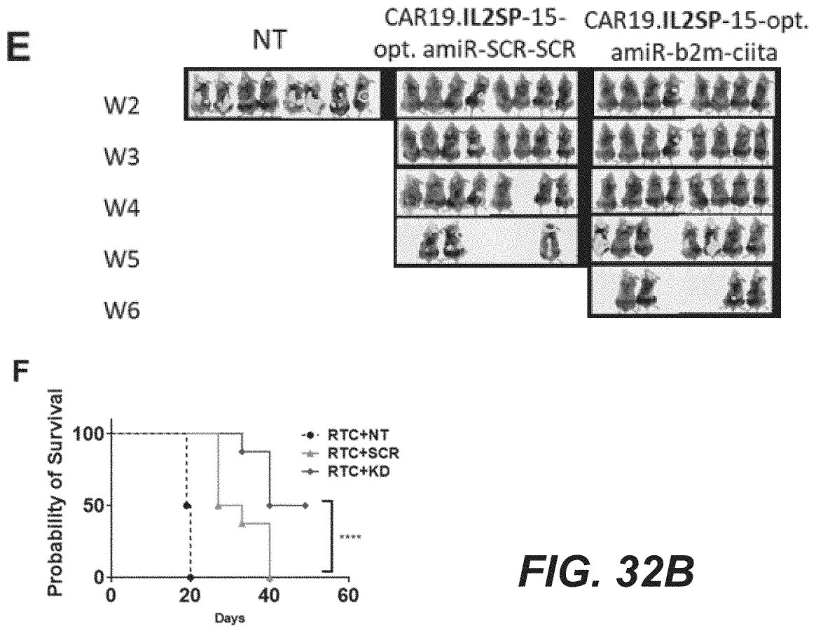

As shown in FIG. 32, NKTs expressing the B2M/CIITA double knockdown construct persist and mediate potent anti-tumor activity in vivo in the presence of allogeneic T cells compared to scrambled control NKTs in the in vivo T cell-mediated rejection model with B cell lymphoma xenograft. NSG mice are irradiated at 1.2 Gy and received intravenously 7×106 in vitro expanded human T cells (days 8-10 postinitial OKT3/αCD28 stimulation) from an HLA-A2-recipient on the following day. One day later, 2×105 firefly luciferase-positive Daudi cells are injected intravenously, followed three days later by 5×106 scrambled control or knockdown transduced NKTs generated from an HLA-A2+ donor. RTC=recipient T cells. B) Representative flow plot showing frequencies of donor HLA-A2+ scrambled control (Scr) or double KD NKT cells in peripheral blood of mice on days 6 and 28. Frequencies of C) HLA-A2+ donor CAR NKT cells and D) HLA-A2− RTCs in peripheral blood after tumor injection. E) Lymphoma progression measured using IVIS imaging at specified time points. F) Kaplan-Meier curve showing survival of mice in each experimental group. P values are determined using two-sided log-rank test. Table 31 below presents the data corresponding to FIG. 32A, panel C. Table 32 below presents the data corresponding to FIG. 32A, panel D. Table 33 presents the data corresponding to FIG. 32B, panel F.

TABLE 31

| | Scramble | | | | | | | |
|---|---|---|---|---|---|---|---|---|
| Day 6 | 1.1 | 0.31 | 1.93 | 1.2 | 2.26 | 2.48 | 3.96 | 0.76 |
| Day 13 | 0.5 | 0.48 | 0.37 | 0.076 | 1.55 | 0.61 | 1.45 | 1.32 |
| Day 19 | 0 | 0 | 0.51 | 0 | 0 | 0.11 | 0.056 | 0.3 |
| Day 28 | 0 | 0.021 | 0.023 | 0.39 | 0 | 0.79 | 0.59 | 0 |
| | KD | | | | | | | |
| Day 6 | 0.02 | 0.16 | 1.44 | 0.25 | 1.65 | 1.02 | 1.24 | 0.76 |
| Day 13 | 0.38 | 2.11 | 0.017 | 0.45 | 0.25 | 0.3 | 0.04 | 0.17 |
| Day 19 | 3.37 | 5.18 | 6.2 | 4.46 | 1.53 | 1.14 | 6.63 | 4.83 |
| Day 28 | 1.64 | 6.01 | 3.97 | 6.97 | 0.43 | 6.93 | 5.42 | 6.24 |

TABLE 32

| | Scramble | | | | | | | |
|---|---|---|---|---|---|---|---|---|
| Day 6 | 18.9 | 4.8 | 15.3 | 6.61 | 6.58 | 3.85 | 10.7 | 24.7 |
| Day 14 | 8.3 | 12 | 7.42 | 0.73 | 2.28 | 1.46 | 3.19 | 2.98 |
| Day 20 | 51.3 | 9.94 | 10.7 | 45.5 | 5.39 | 16 | 30.1 | 12.3 |
| Day 29 | 51.2 | 42.9 | 42.5 | 43.2 | 26.6 | 40.5 | 44.3 | 4.2 |
| | KD | | | | | | | |
| Day 6 | 3.8 | 7.77 | 5.38 | 2.97 | 6.65 | 10.9 | 6.55 | 5.85 |
| Day 14 | 8.45 | 5 | 1.09 | 7.95 | 3.96 | 5.6 | 0.5 | 0.46 |
| Day 20 | 28.4 | 13.3 | 31.6 | 8.46 | 10.4 | 10.8 | | |
| Day 29 | 6.07 | 53 | 54.1 | 51.9 | 55.1 | 49.8 | 40.8 | 43.8 |

TABLE 33

| | | | | | | | | |
|---|---|---|---|---|---|---|---|---|
| | 20 | 20 | 20 | 20 | 19 | 19 | 19 | 19 |
| RTC + NT | 1 | 1 | 1 | 1 | 1 | 1 | 1 | 1 |
| RTC + | | | | | | | | |
| Scramble | | | | | | | | |
| RTC + KD | | | | | | | | |
| | 27 | 40 | 40 | 27 | 33 | 40 | 27 | 27 |
| RTC + NT | | | | | | | | |
| RTC + | 1 | 1 | 1 | 1 | 1 | 1 | 1 | 1 |
| Scramble | | | | | | | | |
| RTC + KD | | | | | | | | |
| | 33 | 49 | 49 | 40 | 49 | 49 | 40 | 40 |
| RTC + NT | | | | | | | | |
| RTC + | | | | | | | | |
| Scramble | | | | | | | | |
| RTC + KD | 1 | 0 | 0 | 1 | 0 | 0 | 1 | 1 |

Taken together, these experiments demonstrate that NKTs expressing the B2M/CIITA double knockdown construct (CAR19.IL2SP-opti15.amiR-B2M-amiR-CIITA) resist killing by allogeneic NK cells while B2M knockout leaves NKTs vulnerable to NK cell killing. NKTs expressing the B2M/CIITA double knockdown construct resist rejection by allogeneic T cells compared to NKTs carrying the scrambled shRNA control construct (CAR19.IL2SP-optil5.amiR-scr-amiR-scr). NKTs expressing the double knockdown construct persist significantly better than scrambled shRNA control NKTs in both T cell and PBMC-mediated in vivo rejection models. NKTs expressing the double knockdown construct retain potent anti-tumor activity in an in vivo T cell-mediated rejection model with Daudi cell xenograft.

### Example 8: amiR Versus Pol III Promoter-Driven shRNA for HLA Class I/II Knockdown and Co-Expression with CAR.GPC3 in NKTS

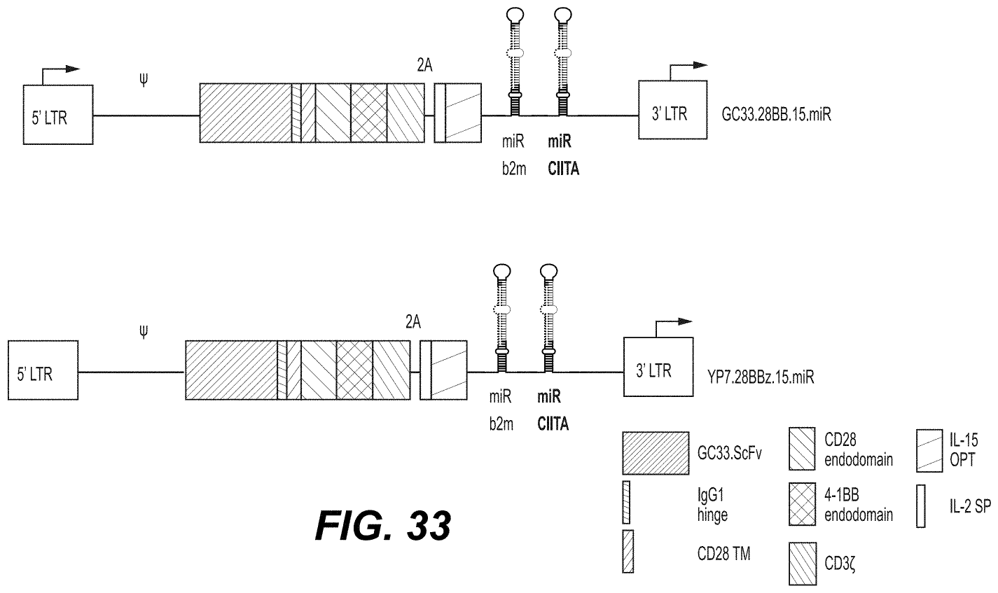

Experiments are carried out to evaluate the feasibility of using amiR scaffolds (e.g., amiR155 and amiR30) to support expression of B2M-shRNA sequences from within CAR.GPC3. A few representative CAR.GPC3 constructs are described, for example, in FIG. 33. The goal is to evaluate how this approach compares to use of polymerase III promoter-driven shRNA in terms of impact on CAR expression and ability to effectively suppress expression of HLA class I and/or II in transduced NKTs.

These experiments are predicted to demonstrate that incorporation of either promoter- or miR-driven shRNA at the 3′ end of the CAR.GPC3 construct similarly reduces the level of CAR expression regardless of shRNA specificity. B2M shRNA expression supported by amiR155 from within CAR.GPC3 yields the greatest level of HLA-A,B,C knockdown compared to the U6, H1, and 7SK polymerase III-driven promoters. The amiR155-B2M shRNA construct mediates more effective and stable suppression of HLA-A,B,C expression compared to the U6-B2M shRNA construct. The amiR30-B2M shRNA construct mediates effective suppression of HLA-A,B,C expression as assessed seven days post-transduction, demonstrating a comparable degree of knockdown to the amiR155-B2M shRNA construct.

### Example 9: Evaluation of Double Knockdown Constructs: amiR-Embedded shRNA Sequences Co-Expressed with CAR.GPC3 and Optimized IL15

To minimize rejection of CAR.GPC3 NKT cells in an allogeneic patient, a construct is designed to knock down HLA class I and II simultaneously using amiR-embedded shRNA sequences to target B2M (class I) and CIITA (class II). The best performing B2M and CIITA-specific shRNAs are selected and evaluated in the single knockdown screening for inclusion in the double knockdown construct: the B2M shRNA target sequence is the same as the one used in the ANCHOR product and is embedded within amiR30, and CIITA shRNA candidate #6 is embedded within amiR155. Codon-optimized IL15 is also integrated to maximize IL15 secretion by NKT cells transduced with this construct based on findings from the previous experiments.

The efficacy of HLA class I and II knockdown mediated by this double knockdown construct is evaluated in transduced NKT cells. An IL15 ELISA is also performed to determine whether the presence of the additional amiR-shRNA impacts IL15 expression or secretion. Additionally, the anti-tumor activity of NKT cells expressing this construct is also evaluated in relevant in vitro and in vivo models.

These experiments are predicted to demonstrate that CAR.GPC3.opti-IL15 double knockdown construct mediates effective HLA class I and II knockdown in NKT cells. NKT cells transduced with CAR.GPC3.opti-IL15 double knockdown construct show similar level of in vitro cytotoxicity against GPC3-positive target cells compared with CAR.GPC3 and CAR.GPC3.IL15 NKTs. NKTs transduced with CAR.GPC3.opti-IL15 double knockdown construct control GPC3+ tumors in vivo and promote survival of NSG mice comparably to CAR.GPC3.15 NKTs. IL15 secretion remains lower in NKTs expressing CAR.GPC3.opti-IL15 double knockdown construct versus original CAR.GPC3.15.

### Example 10: Evaluating Allogenicity of CAR.GPC3 NKTS Expressing Double Knockdown Construct Via Mixed Lymphocyte Reactions (MLR)

To determine how HLA knock-down mediated by the amiR construct impacts NKT allogenicity, several mixed lymphocyte reactions (MLRs) are performed by co-culturing CAR.GPC3.IL2SP-opti15 double knockdown (CAR.GPC3.IL2SP-opti15.amiR-B2M-amiR-CIITA) NKTs with HLA-mismatched NK cells, T cells, or PBMCs. At multiple time-points during co-culture, NKT cell numbers, CAR expression, and HLA expression are evaluated to determine whether the NKTs are able to persist in the presence of allogeneic immune cells. In parallel, the same co-cultures are performed using CAR.GPC3.IL2SP-opti15 NKTs with scrambled shRNA sequences in place of B2M and CIITA shRNA sequences (CAR.GPC3.IL2SP-opti15.amiR-SCR-amiR-SCR), as well as NKTs with B2M and CIITA knocked out (mediated by specific guide RNAs via CRISPR/Cas9).

In addition, several in vivo MLR rejection assays are performed, including allogeneic T cell and PBMC rejection. In these experiments, HLA-mismatched recipient T cells or PBMCs are infused into NSG mice (MHC null in the case of PBMCs) followed four days later by donor NKT cells expressing the amiR double knockdown construct with B2M/CIITA shRNAs or scrambled sequence shRNAs. The T cell rejection model is also evaluated in the context of mice with GPC3+ Daudi lymphoma tumors.

These experiments are predicted to demonstrate that NKTs expressing the B2M/CIITA double knockdown construct (CAR.GPC3.IL2SP-opti15.amiR-B2M-amiR-CIITA) resist killing by allogeneic NK cells while B2M knockout leaves NKTs vulnerable to NK cell killing. NKTs expressing the B2M/CIITA double knockdown construct resist rejection by allogeneic T cells compared to NKTs carrying the scrambled shRNA control construct (CAR.GPC3.IL2SP-opti15.amiR-scr-amiR-scr). NKTs expressing the double knockdown construct persist significantly better than scrambled shRNA control NKTs in both T cell and

PBMC-mediated in vivo rejection models. NKTs expressing the double knockdown construct retain potent anti-tumor activity in an in vivo T cell-mediated rejection model with Daudi cell xenograft.

### Example 11: Evaluating NKT Cells Expressing CAR.GPC3.OPTI-IL15 Double Knockdown Constructs

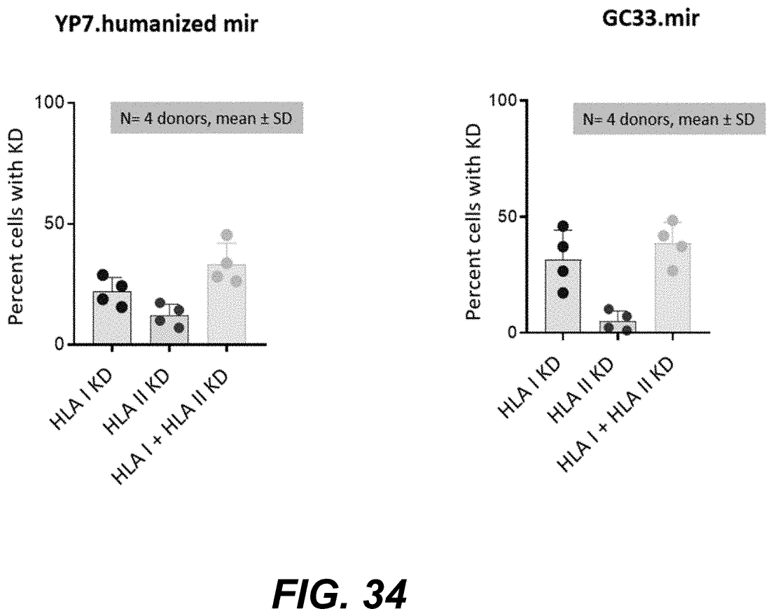

Examples of CAR.GPC3.opti-IL15 double knockdown constructs are shown in FIG. 33. The constructs comprise sequences encoding either the GPC3-specific scFv from GC33 or the scFv from the humanized YP7. FIG. 34 indicates that similar levels of HLA class I or class II gene knockdown are observed in CAR.GPC3 NKT cells expressing either the humanized GPC3 scFv (YP7) or murine GPC3 scFv (GC33).

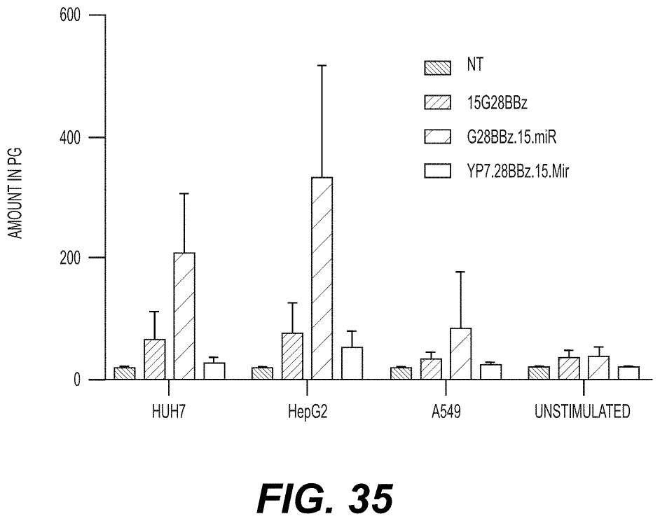

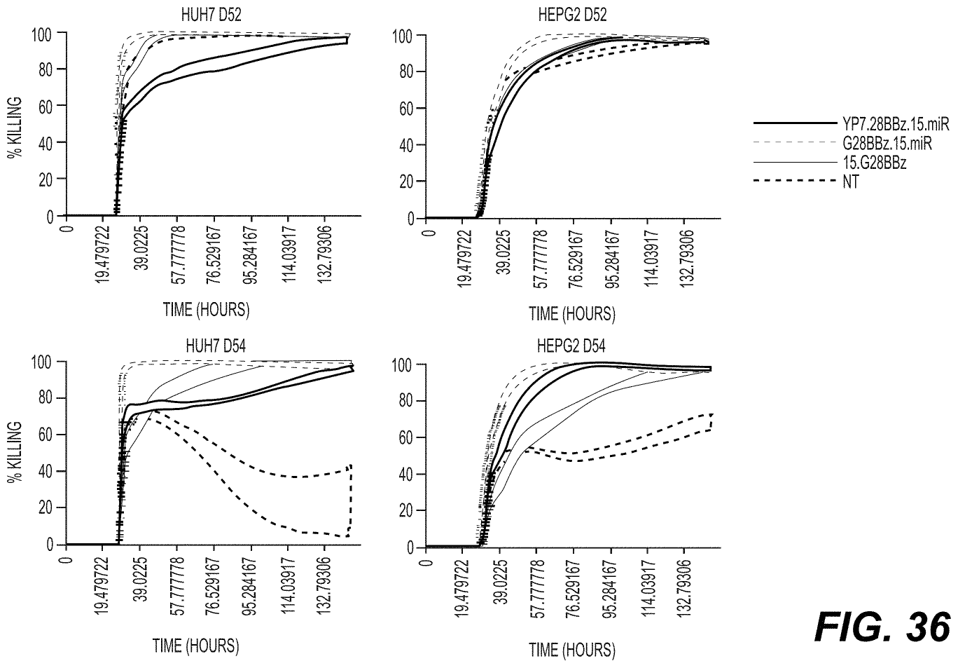

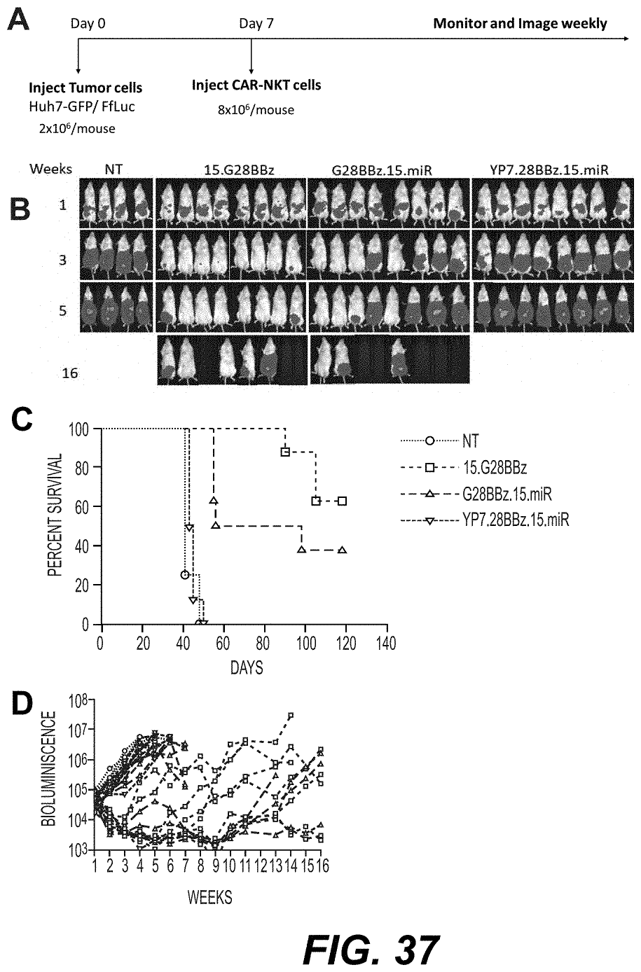

IL-15 production by the CAR.GPC3 NKT cells is measured at baseline (unstimulated) or after stimulation with GPC3-positive Huh-7, HepG2, or A549 cells. FIG. 35 shows that in one experiment, NKT cells expressing murine GPC3 scFv (GC33) double knockdown construct secret more IL-15 than NKT cells expressing humanized GPC3 scFv (YP7) double knockdown construct. Table 34 below presents the data corresponding to FIG. 35. FIG. 36 indicates that in another experiment, NKT cells expressing GC33 double knockdown construct show higher cytotoxicity levels than NKT cells expressing YP7 double knockdown construct, as measured by the xCelligence assay. As indicated in FIG. 37, experiments are carried out to evaluate NKT cells expressing humanized scFv YP7 double knockdown construct or GC33 double knockdown construct in an HCC xenograft model. Table 35 below presents the data corresponding to FIG. 37.

TABLE 34

| | YP7.28BBz.15.miR | | | | G28BBz.15.miR | | | |
|---|---|---|---|---|---|---|---|---|
| HUH7 | 26.82456 | 40.34211 | 20.67544 | 21.67544 | 147.5351 | 290.3158 | 103.3684 | 295.114 |
| HepG2 | 90.03509 | 46.95614 | 24.00877 | 46.09649 | 287.614 | 233.5 | 208.8421 | 607.8421 |
| A549 | 25.99123 | 19.89474 | 19.37719 | 28.94737 | 41.03509 | 45.40351 | 29.42982 | 222.9123 |
| Unstimulated | 20.05263 | 21.24561 | 19.86842 | 21.24561 | 31.26316 | 59.18421 | 22.66667 | 37.15789 |
| | 15G28BBz | | | | NT | | | |
| HUH7 | 133.7193 | 34.70175 | 34.48246 | 60.36842 | 20.55263 | 20.25439 | 20.39474 | 20.75439 |
| HepG2 | 148.2193 | 37.77193 | 50.33333 | 72.52632 | 20.2193 | 19.51754 | 19.99123 | 19.57018 |
| A549 | 45.09649 | 21.00877 | 25.2193 | 42.17544 | 19.88596 | 20.64035 | 19.27193 | 19.14912 |
| Unstimulated | 53.51754 | 24.46491 | 33.29825 | 31.48246 | 19.9386 | 20.35088 | 20.74561 | 20.2807 |

TABLE 35

| | | | | | | | | |
|---|---|---|---|---|---|---|---|---|
| | 41 | 41 | 41 | 48 | | | | |
| NT | 1 | 1 | 1 | 1 | | | | |
| 15.G28BBz | | | | | | | | |
| GC33CAR.15.amiR | | | | | | | | |
| YP7CAR.15.amiR | | | | | | | | |
| | 105 | 105 | 105 | 105 | 105 | 105 | 90 | 105 |
| NT | | | | | | | | |
| 15.G28BBz | 0 | 0 | 1 | 0 | 0 | 0 | 1 | 1 |
| GC33CAR.15.amiR | | | | | | | | |
| YP7CAR.15.amiR | | | | | | | | |
| | 105 | 105 | 98 | 56 | 105 | 55 | 55 | 55 |
| NT | | | | | | | | |
| 15.G28BBz | | | | | | | | |
| GC33CAR.15.amiR | 0 | 0 | 1 | 1 | 0 | 1 | 1 | 1 |
| YP7CAR.15.amiR | | | | | | | | |
| | 50 | 45 | 45 | 45 | 43 | 43 | 43 | 43 |
| NT | | | | | | | | |
| 15.G28BBz | | | | | | | | |
| GC33CAR.15.amiR | | | | | | | | |
| YP7CAR.15.amiR | 1 | 1 | 1 | 1 | 1 | 1 | 1 | 1 |

### Example 12: Positioning IL-15 Upstream of CAR Enhances Transgenic IL-15 Gene Expression

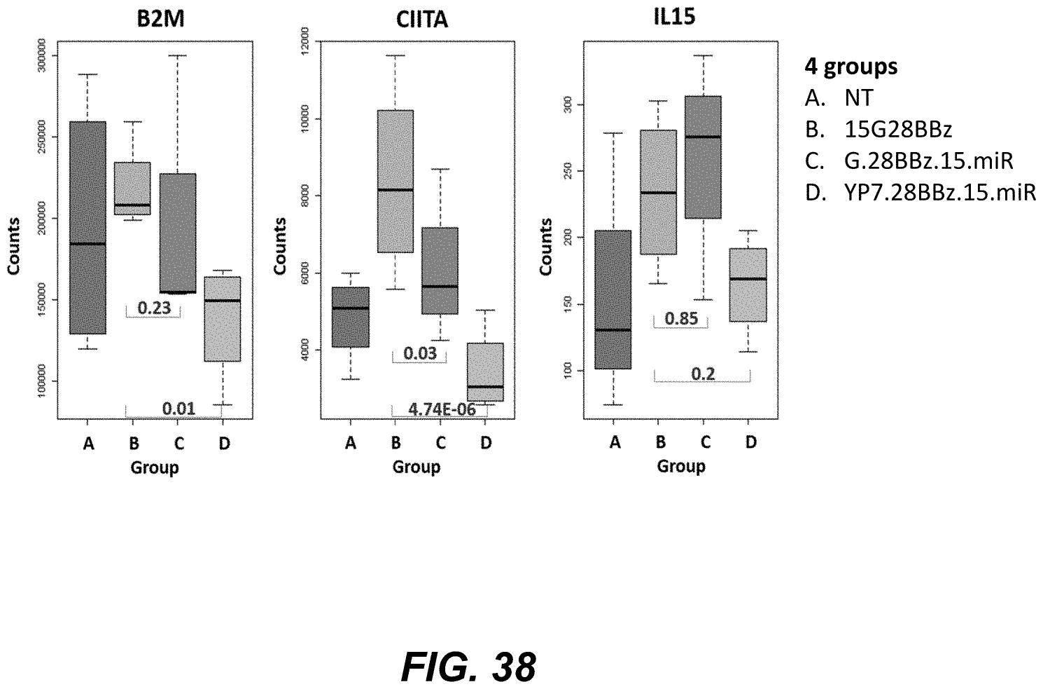

FIG. 38 shows that CAR.GPC3 NKT cells expressing amiR constructs targeting B2M and CIITA express lower levels of these targeted genes, but CAR.GPC3 NKT cells comprising IL15 constructs express higher levels of native IL15. Table 36 below presents the data corresponding to FIG. 38.

TABLE 36

| Gene Symbol | ID | CIITA | B2M | IL15 |
|---|---|---|---|---|
| S1 | A | 3247 | 230091 | 74 |
| S5 | A | 5997 | 288113 | 129 |
| S9 | A | 5278 | 119725 | 132 |
| S12 | A | 4918 | 138546 | 279 |
| S2 | B | 7483 | 206228 | 210 |
| S6 | B | 5584 | 259397 | 303 |
| S10 | B | 8812 | 209223 | 165 |
| S13 | B | 11638 | 198667 | 258 |
| S3 | C | 4248 | 154674 | 153 |
| S7 | C | 8690 | 299664 | 276 |
| S14 | C | 5656 | 153397 | 337 |
| S4 | D | 3300 | 85375 | 114 |
| S8 | D | 2783 | 168190 | 160 |
| S11 | D | 2582 | 139451 | 205 |
| S15 | D | 5051 | 159156 | 178 |

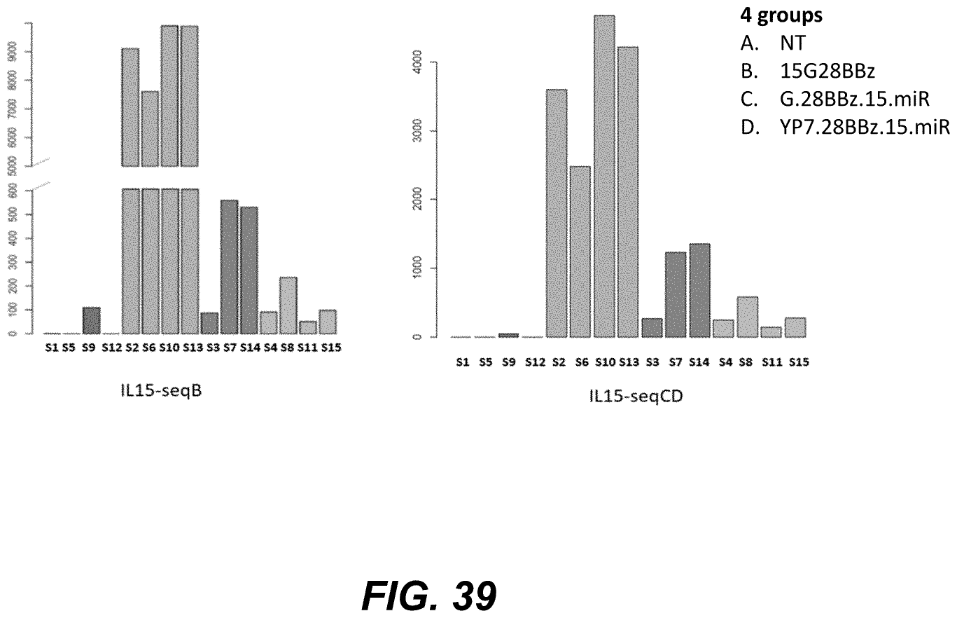

Alternative constructs are prepared to test the effect of positioning IL-15 upstream of CAR.GPC3 on the level of transgenic IL-15 gene expression. FIG. 39 indicates that positioning IL-15 coding sequence upstream of CAR.GPC3 enhances the expression level of transgenic IL-15 gene. Table 37 below presents the data corresponding to FIG. 39.

TABLE 37

| GeneSymbol | Group | IL15-B | IL15-CD |
|---|---|---|---|
| S1 | A | 1 | 0 |
| S5 | A | 0 | 0 |
| S9 | A | 110 | 41 |
| S12 | A | 0 | 0 |
| S2 | B | 8285 | 3599 |
| S6 | B | 5258 | 2482 |
| S10 | B | 9898 | 4675 |
| S13 | B | 9877 | 4220 |
| S3 | C | 87 | 267 |
| S7 | C | 559 | 1232 |
| S14 | C | 530 | 1358 |
| S4 | D | 91 | 245 |
| S8 | D | 236 | 576 |
| S11 | D | 51 | 137 |
| S15 | D | 98 | 274 |

### Example 13: Evaluating the Effect of B2M and CIITA Knockdowns on Global Gene Expression in CAR.GPC3 NKT Cells

Global differential gene expression is analyzed to examine the effect of HLA class I and class II double knockdown by amiRs on humanized YP7 or murine GPC3-expressing CAR.GPC3 NKT cells. Table 38 below summaries the numbers of upregulated or downregulated genes as analyzed in 4 donors.

TABLE 38

| | | | | G.28BBz. |
|---|---|---|---|---|
| | | G28BBz. | YP7.28BBz. | 15.miR vs. |
| | 15.G28BBz | 15.miR vs. | 15.miR vs. | YP7.G28BBz. |
| | vs. NT | 15.G28BBz | 15.G28BBz | 15.miR |
| Up-regulated | 159 | 6 | 36 | 8 |
| Down-regulated | 2 | 43 | 119 | 4 |

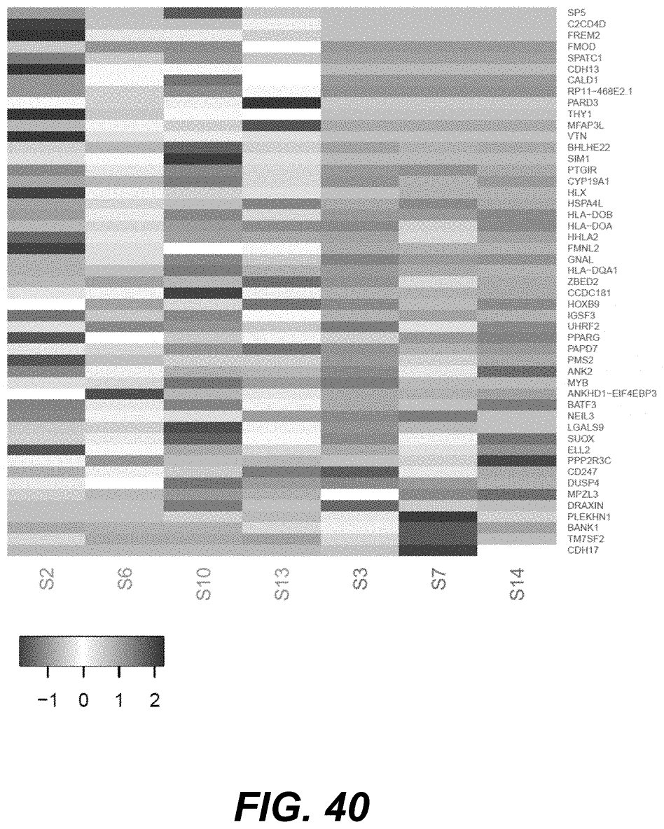

FIG. 40 is a heat map illustrating the HLA-specific genes downregulated in G.28BBz.15.miR-expressing NKT cells in comparison with 15G28BBz-expressing NKT cells. Table 39 below summarizes the negatively regulated genes. Table 40 presents the data corresponding to FIG. 40.

TABLE 39

| pathway | members_input_overlap | p-value | q-value |
|---|---|---|---|
| Asthma - Homo sapiens (human) | HLA-DOA; HLA-DOB; | 4.26E−05 | 0.001424807 |
| | HLA-DQA1 | | |
| Th1 and Th2 cell differentiation - | CD247; HLA-DOA; | 6.64E−05 | 0.001424807 |
| Homo sapiens (human) | HLA-DOB; HLA-DQA1 | | |
| Allograft rejection - Homo sapiens | HLA-DQA1; HLA-DOB; | 8.42E−05 | 0.001424807 |
| (human) | HLA-DOA | | |
| Graft-versus-host disease - Homo | HLA-DQA1; HLA-DOB; | 9.96E−05 | 0.001424807 |
| sapiens (human) | HLA-DOA | | |
| Th17 cell differentiation - Homo | CD247; HLA-DOA; | 0.000121137 | 0.001424807 |
| sapiens (human) | HLA-DOB; HLA-DQA1 | | |
| Type I diabetes mellitus - Homo | HLA-DQA1; HLA-DOB; | 0.000135696 | 0.001424807 |
| sapiens (human) | HLA-DOA | | |
| Intestinal immune network for IgA | HLA-DQA1; HLA-DOB; | 0.000179432 | 0.001614891 |
| production - Homo sapiens (human) | HLA-DOA | | |
| Autoimmune thyroid disease - | HLA-DOA; HLA-DOB; | 0.000245788 | 0.001933526 |
| Homo sapiens (human) | HLA-DQA1 | | |
| Staphylococcus aureus infection - | HLA-DQA1; HLA-DOB; | 0.000276218 | 0.001933526 |
| Homo sapiens (human) | HLA-DOA | | |
| Viral myocarditis - Homo sapiens | HLA-DQA1; HLA-DOB; | 0.000344162 | 0.002077158 |
| (human) | HLA-DOA | | |

TABLE 40

| GeneSymbol | S2 | S6 | S10 | S13 | S3 | S7 | S14 |
|---|---|---|---|---|---|---|---|
| CDH17 | 0 | 0 | 0 | 0 | 9 | 19 | 4 |
| TM7SF2 | 239 | 72 | 74 | 64 | 552 | 1019 | 646 |
| BANK1 | 4 | 8 | 8 | 19 | 27 | 93 | 66 |
| PLEKHN1 | 62 | 66 | 103 | 51 | 146 | 680 | 313 |
| DRAXIN | 2063 | 2070 | 620 | 2004 | 7288 | 4732 | 5162 |
| MPZL3 | 2535 | 2141 | 1322 | 1990 | 3867 | 7004 | 7809 |
| DUSP4 | 19607 | 17684 | 28254 | 25238 | 10657 | 11759 | 13226 |
| CD247 | 51712 | 35620 | 47633 | 60685 | 14951 | 35234 | 26019 |
| PPP2R3C | 2223 | 3000 | 2683 | 2738 | 1567 | 1697 | 617 |
| ELL2 | 16092 | 7184 | 11880 | 5736 | 3884 | 6076 | 3045 |
| SUOX | 390 | 352 | 580 | 324 | 137 | 277 | 108 |
| LGALS9 | 1413 | 745 | 2264 | 1147 | 406 | 614 | 662 |
| NEIL3 | 1247 | 831 | 903 | 1161 | 365 | 306 | 536 |
| BATF3 | 7245 | 3707 | 7222 | 4663 | 2373 | 2963 | 1523 |
| ANKHD1- | 3129 | 6854 | 4695 | 2767 | 1723 | 1848 | 1540 |
| EIF4EBP3 | | | | | | | |
| MYB | 5221 | 3329 | 7780 | 6913 | 1278 | 2686 | 2754 |
| ANK2 | 103 | 55 | 89 | 88 | 27 | 54 | 18 |
| PMS2 | 555 | 153 | 330 | 330 | 99 | 172 | 115 |
| PAPD7 | 1718 | 2468 | 3235 | 3620 | 861 | 1162 | 965 |
| PPARG | 1103 | 513 | 716 | 560 | 376 | 205 | 145 |
| UHRF2 | 1116 | 2341 | 2234 | 1805 | 411 | 1086 | 544 |
| IGSF3 | 145 | 43 | 133 | 75 | 32 | 27 | 31 |
| HOXB9 | 126 | 201 | 159 | 239 | 45 | 75 | 43 |
| CCDC181 | 24 | 21 | 51 | 16 | 7 | 7 | 6 |
| ZBED2 | 14613 | 2244 | 14743 | 20461 | 1309 | 6107 | 2519 |
| HLA-DQA1 | 21103 | 6398 | 26714 | 21053 | 3342 | 4573 | 5088 |
| GNAL | 384 | 316 | 458 | 344 | 67 | 95 | 89 |
| FMNL2 | 269 | 123 | 91 | 85 | 25 | 28 | 33 |
| HHLA2 | 205 | 101 | 154 | 18 | 15 | 51 | 13 |
| HLA-DOA | 1884 | 784 | 2092 | 2235 | 155 | 772 | 170 |
| HLA-DOB | 97 | 62 | 104 | 71 | 17 | 16 | 12 |
| HSPA4L | 40 | 14 | 34 | 46 | 6 | 0 | 5 |
| HLX | 5391 | 1226 | 2504 | 934 | 451 | 180 | 131 |
| CYP19A1 | 103 | 98 | 128 | 34 | 4 | 17 | 8 |
| PTGIR | 749 | 247 | 746 | 477 | 37 | 14 | 85 |
| SIM1 | 378 | 203 | 976 | 146 | 29 | 18 | 16 |
| BHLHE22 | 60 | 238 | 369 | 198 | 4 | 21 | 10 |
| VTN | 35 | 4 | 11 | 4 | 0 | 0 | 0 |
| MFAP3L | 18 | 11 | 4 | 29 | 0 | 0 | 0 |
| THY1 | 1074 | 44 | 273 | 246 | 4 | 8 | 1 |
| PARD3 | 12 | 2 | 7 | 53 | 0 | 0 | 0 |
| RP11-468E2.1 | 24 | 17 | 25 | 12 | 0 | 0 | 0 |
| CALD1 | 27 | 13 | 31 | 12 | 0 | 0 | 0 |
| CDH13 | 53 | 11 | 11 | 13 | 0 | 0 | 0 |
| SPATC1 | 37 | 23 | 33 | 5 | 0 | 0 | 0 |
| FMOD | 30 | 42 | 43 | 19 | 0 | 0 | 0 |
| FREM2 | 109 | 14 | 33 | 4 | 0 | 0 | 0 |
| C2CD4D | 154 | 75 | 0 | 25 | 0 | 0 | 0 |
| SP5 | 168 | 0 | 238 | 4 | 0 | 0 | 0 |

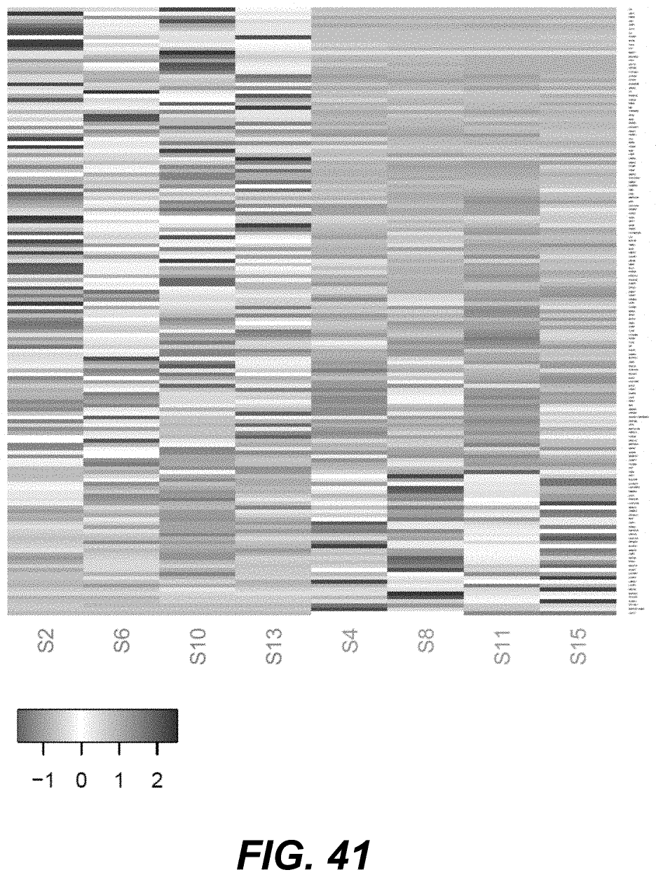

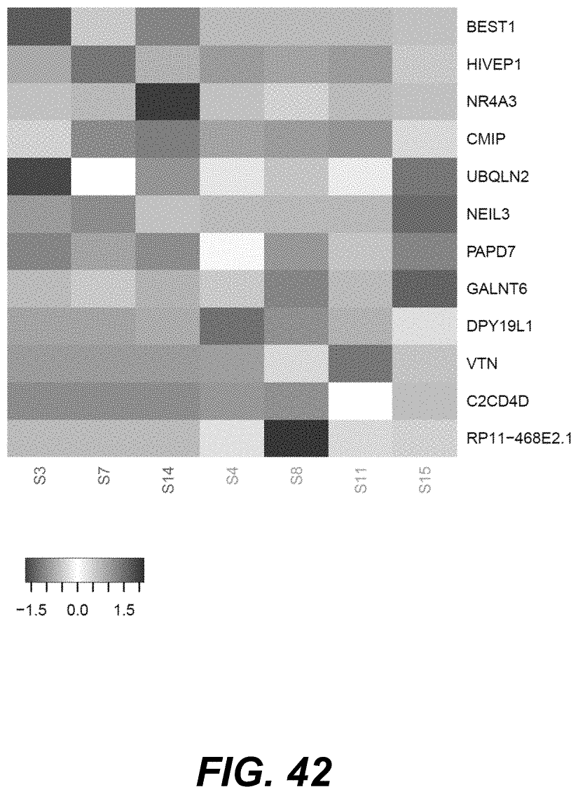

FIG. 41 is a heat map illustrating the HLA-specific and immune effector genes downregulated in YP7.28BBz.15.miR-expressing NKT cells in comparison with 15G28BBz expressing NKT-cells. Table 41 below summarizes the negatively regulated genes. Table 42 below summarizes the positively regulated genes. Table 43 shows presents the data corresponding to FIG. 41.

TABLE 41

| pathway | members_input_overlap | p-value | q-value |
|---|---|---|---|
| Th17 cell differentiation - Homo | IL2RA; HLA-DRA; IL23A; IFNG; | 5.98E−09 | 1.26E−06 |
| sapiens (human) | NFKBIA; NFKBIE; IL21; CD247; | | |
| | HLA-DOA; HLA-DQA1 | | |
| NF-kappa B signaling pathway - | TICAM2; BCL2A1; TAB3; | 3.12E−08 | 3.06E−06 |
| Homo sapiens (human) | CXCL8; NFKBIA; BIRC3; LTA; | | |
| | VCAM1; ICAM1 | | |
| Cytokine Signaling in Immune | IL2RA; HLA-DRA; IL23A; IL3; | 4.36E−08 | 3.06E−06 |
| system | IFNG; CIITA; NFKBIA; BIRC3; | | |
| | CSF2; TAB3; LTA; TNFRSF9; | | |
| | ICAM1; DUSP4; VCAM1; HLA- | | |
| | DQA1; IL9 | | |
| Rheumatoid arthritis - Homo sapiens | HLA-DRA; IL23A; IFNG; CXCL8; | 2.59E−07 | 1.36E−05 |
| (human) | CSF2; HLA-DOA; ICAM1; HLA- | | |
| | DQA1 | | |
| Th1 and Th2 cell differentiation - | IL2RA; HLA-DRA; IFNG; | 3.69E−07 | 1.56E−05 |
| Homo sapiens (human) | NFKBIA; NFKBIE; CD247; HLA- | | |
| | DOA; HLA-DQA1 | | |
| Asthma - Homo sapiens (human) | IL3; HLA-DRA; IL9; HLA-DOA; | 1.91E−06 | 6.70E−05 |
| | HLA-DQA1 | | |
| Viral myocarditis - Homo sapiens | HLA-DRA; DMD; HLA-DOA; | 3.82E−06 | 0.000115012 |
| (human) | ICAM1; MYH6; HLA-DQA1 | | |
| Inflammatory bowel disease (IBD) - | HLA-DRA; IL23A; IFNG; IL21; | 7.65E−06 | 0.000201841 |
| Homo sapiens (human) | HLA-DOA; HLA-DQA1 | | |
| Antigen processing and presentation - | CD74; HLA-DRA; IFNG; CIITA; | 1.20E−05 | 0.000248875 |
| Homo sapiens (human) | HLA-DOA; HLA-DQA1 | | |
| Cytokine-cytokine receptor | IL2RA; CCL1; IL23A; IL3; IFNG; | 1.21E−05 | 0.000248875 |
| interaction - Homo sapiens (human) | CXCL8; CSF2; IL21; LTA; | | |
| | TNFRSF9; IL9 | | |

TABLE 42

| pathway | members_input_overlap | p-value | q-value |
|---|---|---|---|
| Endocytosis - | CXCR1; BIN1; LDLRAP1 | 0.009491706 | 0.151867301 |
| Homo sapiens | | | |
| (human) | | | |

TABLE 43

| GeneSymbol | S2 | S6 | S10 | S13 | S4 | S8 | S11 | S15 |
|---|---|---|---|---|---|---|---|---|
| CDH17 | 0 | 0 | 0 | 0 | 12 | 12 | 34 | 36 |
| BORCS7- | 0 | 0 | 0 | 0 | 47 | 18 | 0 | 14 |
| ASMT | | | | | | | | |
| DPY19L1 | 126 | 4 | 5 | 4 | 2415 | 2039 | 140 | 1267 |
| ALS2CL | 0 | 0 | 8 | 9 | 94 | 12 | 66 | 266 |
| PTCHD2 | 0 | 0 | 4 | 4 | 32 | 56 | 12 | 14 |
| MAP3K2 | 9 | 10 | 10 | 27 | 22 | 706 | 14 | 58 |
| ABLIM1 | 6 | 5 | 38 | 13 | 26 | 28 | 117 | 294 |
| CXCR1 | 73 | 34 | 65 | 95 | 422 | 286 | 564 | 425 |
| CREG2 | 12 | 14 | 19 | 7 | 131 | 61 | 39 | 47 |
| KCNH3 | 19 | 66 | 9 | 42 | 159 | 76 | 75 | 411 |
| CALHM1 | 15 | 12 | 0 | 18 | 57 | 32 | 69 | 64 |
| SYNE1 | 252 | 969 | 370 | 715 | 1751 | 5292 | 2605 | 1805 |
| GALNT6 | 212 | 250 | 188 | 110 | 204 | 1560 | 145 | 1795 |
| RHOU | 187 | 804 | 344 | 633 | 553 | 3852 | 1825 | 3158 |
| WNT9A | 26 | 79 | 18 | 83 | 192 | 329 | 151 | 192 |
| LIME1 | 783 | 1290 | 455 | 964 | 3441 | 4105 | 2360 | 4283 |
| SMAD6 | 144 | 86 | 57 | 41 | 287 | 359 | 174 | 425 |
| ROPN1L | 155 | 147 | 77 | 102 | 758 | 314 | 237 | 269 |
| PPFIBP2 | 314 | 129 | 176 | 278 | 923 | 562 | 616 | 807 |
| KIAA1161 | 258 | 250 | 191 | 350 | 362 | 1091 | 487 | 1469 |
| DRAXIN | 2063 | 2070 | 620 | 2004 | 5183 | 5485 | 2793 | 6940 |
| ZMYND10 | 71 | 86 | 37 | 32 | 229 | 220 | 98 | 116 |
| RASA3 | 5373 | 2654 | 2746 | 3478 | 12886 | 7941 | 5542 | 13030 |
| CRIP1 | 8447 | 8580 | 5222 | 4614 | 25459 | 14290 | 15545 | 13572 |
| BIN1 | 1975 | 3441 | 1440 | 2082 | 4006 | 7502 | 6805 | 4579 |
| ZFP36L2 | 7709 | 7238 | 6065 | 9888 | 18900 | 23452 | 12636 | 25712 |
| TREML2 | 652 | 1023 | 647 | 1393 | 1487 | 2729 | 2103 | 3264 |
| SAMD3 | 1518 | 2075 | 1669 | 921 | 3746 | 4847 | 3989 | 2602 |
| CCDC74B | 207 | 81 | 108 | 135 | 216 | 335 | 215 | 576 |
| FAM227B | 47 | 25 | 19 | 49 | 73 | 99 | 58 | 94 |
| ADD1 | 7049 | 3068 | 3968 | 5837 | 12100 | 13632 | 6838 | 13396 |
| RIMBP3 | 103 | 85 | 45 | 68 | 150 | 257 | 101 | 187 |
| CDC42EP4 | 209 | 109 | 104 | 128 | 212 | 507 | 184 | 355 |
| LDLRAP1 | 1249 | 560 | 935 | 758 | 2266 | 1316 | 1658 | 2585 |
| ANXA2R | 511 | 728 | 285 | 404 | 894 | 1204 | 676 | 1400 |
| GPC1 | 534 | 682 | 456 | 554 | 857 | 1946 | 794 | 1208 |
| TAB3 | 400 | 435 | 316 | 460 | 247 | 198 | 105 | 279 |
| OAF | 0 | 3 | 4 | 4 | 0 | 0 | 0 | 0 |
| TICAM2 | 227 | 403 | 347 | 325 | 161 | 213 | 141 | 139 |
| NAMPT | 9663 | 13886 | 12885 | 11718 | 5099 | 7594 | 5532 | 5841 |
| MAMLD1 | 2898 | 1841 | 2660 | 2549 | 1701 | 940 | 889 | 1246 |
| AKAP5 | 987 | 947 | 1491 | 1501 | 478 | 539 | 598 | 797 |
| WARS | 24766 | 14365 | 25449 | 17206 | 10344 | 9047 | 11869 | 7277 |
| SERTAD2 | 6971 | 5747 | 3912 | 6029 | 2555 | 3121 | 1676 | 2822 |
| GPR137 | 681 | 1444 | 554 | 965 | 483 | 447 | 286 | 392 |
| CCDC6 | 4393 | 4811 | 6413 | 8606 | 2024 | 2128 | 2519 | 4053 |
| NFE2L3 | 12865 | 10202 | 11739 | 14729 | 3915 | 6624 | 4013 | 6250 |
| MAP1LC3A | 812 | 775 | 675 | 564 | 257 | 324 | 274 | 311 |
| CIITA | 7483 | 5584 | 8812 | 11638 | 3300 | 2783 | 2582 | 5051 |
| RNF19B | 7244 | 4981 | 5595 | 5980 | 2026 | 2707 | 1726 | 3166 |
| ANKHD1- | 3129 | 6854 | 4695 | 2767 | 2530 | 992 | 1001 | 2522 |
| EIF4EBP3 | | | | | | | | |
| ZNF629 | 258 | 192 | 210 | 376 | 58 | 150 | 91 | 119 |
| ARID5A | 10976 | 4993 | 9514 | 8795 | 1751 | 3157 | 2311 | 6811 |
| MAL | 10493 | 11520 | 7313 | 8301 | 3180 | 4249 | 2530 | 5156 |
| RND1 | 327 | 299 | 239 | 209 | 65 | 177 | 89 | 95 |
| CD74 | 290136 | 323828 | 242139 | 257827 | 76961 | 185208 | 75921 | 105828 |
| DUSP5 | 14074 | 17487 | 11744 | 11812 | 4620 | 8062 | 4270 | 4529 |
| CD247 | 51712 | 35620 | 47633 | 60685 | 11656 | 28131 | 14750 | 22492 |
| SUOX | 390 | 352 | 580 | 324 | 118 | 104 | 230 | 168 |
| HLA-DRA | 48324 | 50992 | 37979 | 42365 | 13244 | 28314 | 13381 | 12193 |
| ANK2 | 103 | 55 | 89 | 88 | 36 | 35 | 29 | 21 |
| BCL2A1 | 497 | 860 | 968 | 485 | 207 | 391 | 236 | 179 |
| ZC3H12A | 2954 | 3605 | 2365 | 2721 | 540 | 1030 | 1153 | 1477 |
| RDH10 | 5035 | 6521 | 9933 | 5347 | 1875 | 2756 | 3076 | 1720 |
| CD83 | 1465 | 2081 | 2255 | 1362 | 451 | 1076 | 492 | 518 |
| ENTPD1 | 7017 | 12878 | 6229 | 6319 | 2220 | 3565 | 2220 | 3450 |
| DUSP4 | 19607 | 17684 | 28254 | 25238 | 8356 | 6108 | 7346 | 9524 |
| ADAP1 | 1772 | 1782 | 3148 | 3100 | 732 | 538 | 1052 | 1028 |
| GK | 5150 | 3558 | 3616 | 3497 | 1968 | 1213 | 1040 | 1023 |
| TTC8 | 159 | 123 | 201 | 136 | 89 | 32 | 27 | 56 |
| RHOB | 1197 | 817 | 1124 | 1116 | 395 | 343 | 153 | 481 |
| C17orf96 | 2027 | 1639 | 2887 | 3050 | 582 | 973 | 386 | 1195 |
| IL23A | 1816 | 1463 | 886 | 2073 | 489 | 513 | 277 | 704 |
| IL2RA | 93734 | 65613 | 73811 | 64818 | 28866 | 22177 | 20536 | 19521 |
| RGS1 | 5172 | 2986 | 4385 | 2412 | 1494 | 732 | 870 | 1419 |
| BATF3 | 7245 | 3707 | 7222 | 4663 | 1622 | 2187 | 1792 | 1197 |
| SDC4 | 15444 | 13101 | 16091 | 19612 | 3822 | 5355 | 3449 | 6958 |
| BIRC3 | 39671 | 22881 | 26471 | 24648 | 10949 | 12355 | 3885 | 6282 |
| HOXB9 | 126 | 201 | 159 | 239 | 58 | 35 | 32 | 92 |
| UCP3 | 920 | 207 | 324 | 110 | 109 | 180 | 56 | 91 |
| NFKBIA | 31278 | 37481 | 26306 | 26912 | 5043 | 14484 | 6235 | 9266 |
| ICAM1 | 12109 | 10015 | 7496 | 6918 | 2534 | 4095 | 1823 | 1472 |
| ZMIZ2 | 9418 | 12861 | 7177 | 8129 | 3770 | 2279 | 2025 | 1699 |
| ZFHX2 | 501 | 212 | 208 | 332 | 118 | 65 | 75 | 49 |
| DUSP2 | 1060 | 1131 | 2242 | 1569 | 220 | 380 | 441 | 431 |
| POLR1B | 872 | 506 | 1170 | 539 | 213 | 177 | 108 | 255 |
| AFAP1L2 | 1389 | 1873 | 2090 | 1985 | 438 | 583 | 251 | 538 |
| PPARG | 1103 | 513 | 716 | 560 | 288 | 154 | 104 | 124 |
| ELL2 | 16092 | 7184 | 11880 | 5736 | 2913 | 1890 | 1637 | 2793 |
| G0S2 | 857 | 269 | 264 | 522 | 185 | 109 | 23 | 115 |
| ABTB2 | 676 | 306 | 1523 | 396 | 289 | 69 | 190 | 81 |
| CXorf21 | 564 | 658 | 316 | 641 | 69 | 167 | 51 | 225 |
| INSIG1 | 8566 | 1623 | 3828 | 3481 | 766 | 1434 | 727 | 931 |
| MYB | 5221 | 3329 | 7780 | 6913 | 1363 | 984 | 1185 | 1587 |
| HMSD | 291 | 252 | 116 | 174 | 49 | 88 | 23 | 10 |
| MYO1B | 503 | 220 | 207 | 153 | 65 | 67 | 36 | 46 |
| LTA | 7883 | 11312 | 21272 | 8862 | 1035 | 2930 | 2872 | 2947 |
| CTTNBP2NL | 761 | 391 | 251 | 466 | 70 | 190 | 8 | 72 |
| NR4A1 | 952 | 655 | 1084 | 1368 | 226 | 203 | 111 | 163 |
| GHRL | 32 | 59 | 73 | 190 | 11 | 15 | 7 | 29 |
| GGT1 | 2971 | 367 | 830 | 1364 | 342 | 93 | 221 | 203 |
| IQCG | 2816 | 543 | 578 | 241 | 205 | 107 | 207 | 94 |
| NTRK1 | 445 | 390 | 631 | 779 | 52 | 67 | 58 | 188 |
| NFKBIE | 1312 | 1652 | 2230 | 694 | 108 | 137 | 95 | 633 |
| HLA-DOA | 1884 | 784 | 2092 | 2235 | 53 | 414 | 157 | 490 |
| SPR | 64 | 35 | 74 | 43 | 20 | 8 | 0 | 4 |
| SMPDL3A | 211 | 140 | 125 | 166 | 30 | 21 | 11 | 34 |
| CPM | 639 | 162 | 407 | 308 | 91 | 23 | 28 | 67 |
| DMD | 641 | 2689 | 840 | 3201 | 190 | 136 | 62 | 699 |
| ADGRE1 | 727 | 130 | 624 | 268 | 85 | 27 | 34 | 89 |
| ZBED2 | 14613 | 2244 | 14743 | 20461 | 1268 | 1974 | 1658 | 2062 |
| HLA-DQA1 | 21103 | 6398 | 26714 | 21053 | 2874 | 1206 | 2229 | 3609 |
| SGPP2 | 317 | 570 | 718 | 561 | 73 | 78 | 49 | 72 |
| TRIB1 | 926 | 492 | 1062 | 1510 | 124 | 85 | 87 | 199 |
| PTGIR | 749 | 247 | 746 | 477 | 95 | 7 | 20 | 154 |
| NR4A2 | 641 | 691 | 648 | 957 | 131 | 97 | 49 | 73 |
| USP9X | 859 | 393 | 2461 | 9343 | 325 | 395 | 304 | 558 |
| CSF2 | 16823 | 11511 | 16042 | 14243 | 2698 | 1080 | 1879 | 597 |
| SIM1 | 378 | 203 | 976 | 146 | 32 | 27 | 55 | 78 |
| CD200 | 1531 | 194 | 591 | 166 | 175 | 24 | 39 | 8 |
| EGR2 | 1801 | 2304 | 4272 | 3959 | 256 | 449 | 328 | 288 |
| HLX | 5391 | 1226 | 2504 | 934 | 629 | 76 | 47 | 245 |
| HIVEP1 | 1089 | 947 | 706 | 961 | 43 | 65 | 42 | 229 |
| PMCH | 23 | 68 | 55 | 65 | 0 | 4 | 8 | 9 |
| POU2AF1 | 1107 | 232 | 1316 | 594 | 96 | 22 | 119 | 62 |
| RASD2 | 43 | 102 | 89 | 124 | 5 | 11 | 4 | 12 |
| GNE | 94 | 832 | 473 | 359 | 21 | 17 | 65 | 43 |
| IFNG | 8790 | 40287 | 17188 | 15533 | 405 | 3431 | 1253 | 1416 |
| TNFRSF9 | 2575 | 2616 | 3220 | 1891 | 187 | 245 | 98 | 268 |
| MB | 111 | 104 | 73 | 335 | 8 | 26 | 2 | 11 |
| RRAD | 435 | 369 | 839 | 439 | 45 | 32 | 49 | 13 |
| CCDC3 | 1613 | 176 | 402 | 1052 | 152 | 0 | 23 | 35 |
| PIP5K1B | 493 | 226 | 186 | 782 | 21 | 34 | 48 | 5 |
| C3 | 688 | 5818 | 637 | 1240 | 55 | 274 | 22 | 246 |
| NR4A3 | 1132 | 1341 | 1737 | 1857 | 85 | 134 | 59 | 77 |
| KIAA0226L | 677 | 65 | 317 | 213 | 35 | 0 | 0 | 24 |
| CXCL8 | 3731 | 5098 | 4341 | 5793 | 172 | 160 | 231 | 338 |
| CYP7B1 | 64 | 80 | 65 | 114 | 0 | 5 | 0 | 10 |
| CYP19A1 | 103 | 98 | 128 | 34 | 4 | 3 | 4 | 4 |
| CRTAM | 3040 | 2084 | 5071 | 1948 | 30 | 157 | 98 | 142 |
| DACT3 | 85 | 48 | 163 | 59 | 11 | 0 | 0 | 0 |
| CCL1 | 9251 | 9069 | 8890 | 1933 | 126 | 405 | 105 | 65 |
| BHLHE22 | 60 | 238 | 369 | 198 | 8 | 12 | 1 | 0 |
| BEST1 | 6 | 19 | 1322 | 18 | 10 | 1 | 2 | 19 |
| IL21 | 406 | 146 | 249 | 155 | 10 | 0 | 5 | 4 |
| THY1 | 1074 | 44 | 273 | 246 | 22 | 0 | 0 | 0 |
| MYH6 | 481 | 109 | 183 | 100 | 4 | 3 | 0 | 5 |
| VCAM1 | 37 | 79 | 19 | 151 | 0 | 0 | 0 | 4 |
| IL3 | 9379 | 3195 | 3245 | 1302 | 96 | 45 | 11 | 60 |
| CA12 | 177 | 32 | 91 | 70 | 3 | 0 | 0 | 0 |
| XIRP1 | 400 | 178 | 302 | 189 | 0 | 4 | 0 | 4 |
| TIE1 | 330 | 109 | 515 | 39 | 0 | 0 | 0 | 7 |
| FMOD | 30 | 42 | 43 | 19 | 0 | 0 | 0 | 0 |
| HEY1 | 92 | 18 | 26 | 10 | 0 | 0 | 0 | 0 |
| IL9 | 45 | 7 | 103 | 28 | 0 | 0 | 0 | 0 |

FIG. 42 is a heat map illustrating that no significant pathways are enriched in humanized YP7.28BBz.15.miR-expressing NKT cells in comparison with murine G.28BBz.15.miR-expressing NKT cells. Table 44 presents the data corresponding to FIG. 42.

TABLE 44

| GeneSymbol | S3 | S7 | S14 | S4 | S8 | S11 | S15 |
|---|---|---|---|---|---|---|---|
| RP11-468E2.1 | 0 | 0 | 0 | 30 | 262 | 104 | 20 |
| C2CD4D | 0 | 0 | 0 | 78 | 79 | 38 | 63 |
| VTN | 0 | 0 | 0 | 10 | 3 | 12 | 8 |
| DPY19L1 | 37 | 10 | 84 | 2415 | 2039 | 140 | 1267 |
| GALNT6 | 123 | 208 | 101 | 204 | 1560 | 145 | 1795 |
| PAPD7 | 861 | 1162 | 965 | 2201 | 3844 | 3238 | 4182 |
| NEIL3 | 365 | 306 | 536 | 489 | 1141 | 1128 | 1484 |
| UBQLN2 | 1514 | 4931 | 3002 | 5510 | 6156 | 5323 | 7835 |
| CMIP | 4259 | 5880 | 6117 | 1516 | 1373 | 1287 | 2467 |
| NR4A3 | 81 | 677 | 1264 | 85 | 134 | 59 | 77 |
| HIVEP1 | 866 | 1122 | 855 | 43 | 65 | 42 | 229 |
| BEST1 | 1034 | 46 | 851 | 10 | 1 | 2 | 19 |

Taken together, these data show lower levels of HLA-specific gene expression in 15G28BBz NKT cells in comparison with NKT cells expressing CARs with B2M/CIITA-specific amiR-shRNAs.

1. A recombinant construct for suppressing the expression of an endogenous major histocompatibility complex (MHC) gene, comprising a DNA sequence encoding a chimeric antigen receptor (CAR) recognizing a tumor antigen and a DNA sequence encoding a small hairpin RNA (shRNA) sequence targeting an MHC class I or MHC class II gene, wherein the shRNA sequence is embedded in an artificial microRNA (amiR) scaffold.

2. The recombinant construct of claim 1, wherein the tumor antigen is CD19, GD2, or GPC3.

3. The recombinant construct of claim 1, further comprising a DNA sequence encoding a cytokine.

4. The recombinant construct of claim 3, wherein the cytokine is interleukin-15 (IL-15), IL-7, IL-12, IL-18, IL-21, IL-27, IL-33, or a combination thereof.

5. The recombinant construct of claim 4, wherein the cytokine is IL-15.

6. The recombinant construct of claim 5, wherein the DNA sequence encoding an IL-15 is codon-optimized.

7. The recombinant construct of claim 5, wherein the IL-15 comprises an IL-2 signal peptide.

8. The recombinant construct of claim 1, wherein the amiR is amiR155 or amiR30.

9. The recombinant construct of claim 1, wherein the shRNA sequence is at least 21 nucleotide in length and comprises a nucleotide sequence identical or complementary to at least 21 contiguous nucleotides of the MHC gene sequence.

10. The recombinant construct of claim 1, wherein the MHC class I gene encodes a β2-microglobulin (B2M).

11. The recombinant construct of claim 1, wherein the MHC class II gene encodes an invariant chain (Ii) or a class II transactivator (CIITA).

12. The recombinant construct of claim 1, wherein the construct comprise a first shRNA sequence embedded in a first amiR scaffold and a second shRNA sequence embedded in a second amiR scaffold.

13. The recombinant construct of claim 12, wherein the first shRNA sequence targets a MHC class I gene and the second shRNA sequence targets a MHC class II gene.

14. The recombinant construct of claim 12, wherein the first amiR scaffold and the second amiR scaffold are from the same amiR sequence or from different amiR sequences.

15. (canceled)

16. A method for limiting rejection of an engineered natural killer T (NKT) cell by the immune system of an allogeneic host, comprising transducing an NKT cell with the recombinant construct of claim 1, wherein the expression of the endogenous MHC gene in the NKT cell is suppressed by the shRNA.

17. The method of claim 16, wherein the expression level of the endogenous MHC gene is decreased by at least 10% 2 days post-transduction, 7 days post-transduction, or 14 days post-transduction.

18. (canceled)

19. (canceled)

20. The method of claim 16, wherein the NKT cell is a CD1d-restrictive NKT cell.

21. An engineered NKT cell, transduced with the recombinant construct of claim 1, wherein the expression of the endogenous MHC gene in the NKT cell is significantly suppressed compared with a control NKT cell not transduced with the recombinant construct.

22. The engineered NKT cell of claim 21, wherein the engineered NKT cell has improved resistance to rejection by allogeneic T cells or PBMCs, or wherein the engineered NKT cell has improved resistance to destruction by allogeneic natural killer cells.

23. (canceled)

24. The engineered NKT cell of claim 21, wherein the engineered NKT cell exhibits anti-tumor activity in vivo.


SEQUENCE LISTING (35 sequences)

SEQ ID NO: 1 - DNA, 21 bp, Homo sapiens
ctggtctttctatctcttgta

SEQ ID NO: 2 - DNA, 21 bp, Homo sapiens
cagcagagaatggaaagtcaa

SEQ ID NO: 3 - DNA, 21 bp, Homo sapiens
ccgtgtgaaccatgtgacttt

SEQ ID NO: 4 - DNA, 21 bp, Homo sapiens
agttaagcgtgcataagttaa

SEQ ID NO: 5 - DNA, 21 bp, Homo sapiens
tagagtttggctcacagtgta

SEQ ID NO: 6 - DNA, 21 bp, Homo sapiens
aggtttgaagatgccgcattt

SEQ ID NO: 7 - DNA, 21 bp, Homo sapiens
ttgtacaagcttagcctgagc

SEQ ID NO: 8 - DNA, 21 bp, Homo sapiens
tagggtactttgatgtctgcg

SEQ ID NO: 9 - DNA, 21 bp, Homo sapiens
gttaagaagctccaggtagcc

SEQ ID NO: 10 - DNA, 21 bp, Homo sapiens
ttccatgtcacacaacagcct

SEQ ID NO: 11 - DNA, 21 bp, Homo sapiens
tttggaagcttgttggagacc

SEQ ID NO: 12 - DNA, 21 bp, Homo sapiens
tttgtaggcacccaggtcagt

SEQ ID NO: 13 - DNA, 21 bp, Homo sapiens
atctcaggctgatccgtgaat

SEQ ID NO: 14 - DNA, 21 bp, Homo sapiens
tggagaagtactttctctgtg

SEQ ID NO: 15 - DNA, 21 bp, Homo sapiens
ttagctgtttccctgctaagg

SEQ ID NO: 16 - DNA, 21 bp, Homo sapiens
tgaactcaaaccctggacctg

SEQ ID NO: 17 - DNA, 21 bp, Homo sapiens
ccaccaagtatggcaacatga

SEQ ID NO: 18 - DNA, 21 bp, Homo sapiens
ccacacagctacagctttctt

SEQ ID NO: 19 - DNA, 21 bp, Homo sapiens
caagtcggaacagcagataac

SEQ ID NO: 20 - DNA, 21 bp, Homo sapiens
cgcgaccttatctccaacaat

SEQ ID NO: 21 - DNA, 21 bp, Homo sapiens
gaccatagactggaaggtctt

SEQ ID NO: 22 - DNA, 21 bp, Homo sapiens
cctttgtagctttcacttcca

SEQ ID NO: 23 - DNA, 21 bp, Homo sapiens
gaacctgagacaccttaagaa

SEQ ID NO: 24 - DNA, 21 bp, Homo sapiens
gcaccattggctcctgtttga

SEQ ID NO: 25 - DNA, 21 bp, Homo sapiens
tcacagcagcctccaacacaa

SEQ ID NO: 26 - DNA, 21 bp, Homo sapiens
caacacaaggctccaagacct

SEQ ID NO: 27 - DNA, 26 bp, Artificial
  Chimeric sequences from human with bacterial vector
tgccatggagtttgggctgagctggc

SEQ ID NO: 28 - DNA, 21 bp, Artificial
  Chimeric sequences from human with bacterial vector
gcgagggggcagggcctgcat

SEQ ID NO: 29 - DNA, 558 bp, Artificial
  Chimeric sequences from human with bacterial vector
cagtgtactaattatgctctcttgaaattggctggagatgttgagagcaatcccgggcccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgagagcgcttg

SEQ ID NO: 30 - DNA, 348 bp, Artificial
  Chimeric sequences from human with bacterial vector
agagcgcttgtttgaatgaggcttcagtactttacagaatcgttgcctgcacatcttggaaacacttgctgggattacttcgacttcttaacccaacagaaggctcgagaaggtatattgctgttgacagtgagcgaaggtttgaagatgccgcattttagtgaagccacagatgtaaaatgcggcatcttcaaacctctgcctactgcctcggacttcaaggggctagaattcgagcaattatcttgtttactaaaactgaataccttgctatctctttgatacatttttacaaagctgaattaaaatggtataaattaaatcactttgttaacatgatgtcgacct

SEQ ID NO: 31 - DNA, 156 bp, Artificial
  Chimeric sequences from human with bacterial vector
atgtcgacctggaggcttgctgaaggctgtatgctgtttgtaggcacccaggtcagtgttttggccactgactgacactgacctgtgcctacaaacaggacacaaggcctgttactagcactcacatggaacaaatggccgtcgacacctcgagat

SEQ ID NO: 32 - DNA, 19 bp, Artificial
  Cloning vector
caccgccctcaaagtagac

SEQ ID NO: 33 - DNA, 22 bp, Artificial
  Cloining vector
atggcctttaccagggtctcag

SEQ ID NO: 34 - DNA, 22 bp, Artificial
  Cloning vector
cgaggaactggaagagaagaat

SEQ ID NO: 35 - DNA, 21 bp, Artificial
  Cloning vector
tcgtactctataggcttcagc